(19) United States
(12) Patent Application Publication
(10) Pub. No.: US 2022/0193076 A1
(43) Pub. Date: Jun. 23, 2022

(54) METHODS OF TREATING BREAST CANCER WITH TUCATINIB

(71) Applicant: Seagen Inc., Bothell, WA (US)

(72) Inventors: Luke WALKER, Bothell, WA (US); Christopher J. ENDRES, Bothell, WA (US); Anthony Jaeyong LEE, Bothell, WA (US); Hao SUN, Bothell, WA (US); Ariel R. TOPLETZ-ERICKSON, Bothell, WA (US)

(21) Appl. No.: 17/310,212

(22) PCT Filed: Jan. 24, 2020
(86) PCT No.: PCT/US2020/014953

§ 371 Date: Jul. 26, 2021

Related U.S. Application Data
(60) Provisional application No. 62/797,854, filed on Jan. 28, 2019.

(51) Int. Cl. A61K 31/517; A61K 31/155; A61K 31/7068; A61K 39/395

(52) U.S. Cl. CPC A61K 31/517; A61K 39/3955; A61K 31/7068; A61K 31/155

(57) ABSTRACT

The invention provides tucatinib and its use in methods of treating cancer, such as breast cancer. The invention also provides compositions and kits comprising tucatinib for use in treating cancer, such as breast cancer.

### CROSS-REFERENCE TO RELATED APPLICATION

This application claims priority to U.S. Provisional Application No. 62/797,854 filed on Jan. 28, 2019, the content of which is incorporated herein by reference in its entirety.

### TECHNICAL FIELD

The present invention relates to methods of treating breast cancer, such as HER2 positive breast cancer, with tucatinib, or salt or solvate thereof.

### BACKGROUND

Breast cancer is by far the most common cancer among women. Each year, more than 180,000 and 1 million women in the U.S. and worldwide, respectively, are diagnosed with breast cancer. Breast cancer is the leading cause of death for women between ages 50-55, and is the most common non-preventable malignancy in women in the Western Hemisphere. An estimated 2,167,000 women in the United States are currently living with the disease. Based on cancer rates from 1995 through 1997, a report from the National Cancer Institute (NCI) estimates that about 1 in 8 women in the United States (approximately 12.8 percent) will develop breast cancer during her lifetime (NCI's Surveillance, Epidemiology, and End Results Program (SEER) publication SEER Cancer Statistic's Review 1973-1997). Breast cancer is the second most common form of cancer, after skin cancer, among women in the United States. An estimated 250,100 new cases of breast cancer are expected to be diagnosed in the United States in 2001 Of these, 192,200 new cases of more advanced (invasive) breast cancer are expected to occur among women (an increase of 5% over last year), 46,400 new cases of early stage (in situ) breast cancer are expected to occur among women (up 9% from last year), and about 1,500 new cases of breast cancer are expected to be diagnosed in men (Cancer Facts & FIGS. 2001 American Cancer Society). An estimated 40,600 deaths (40,300 women, 400 men) from breast cancer are expected in 2001. Breast cancer ranks second only to lung cancer among causes of cancer deaths in women. Nearly 86% of women who are diagnosed with breast cancer are likely to still be alive five years later, though 24% of them will die of breast cancer after 10 years, and nearly half (47%) will die of breast cancer after 20 years.

Every woman is at risk for breast cancer. Over 70 percent of breast cancers occur in women who have no identifiable risk factors other than age (U.S. General Accounting Office. Breast Cancer, 1971-1991: Prevention, Treatment and Research. GAO/PEMhD-92-12; 1991). Only 5 to 10% of breast cancers are linked to a family history of breast cancer (Henderson I C, Breast Cancer. In: Murphy G P, Lawrence W L, Lenhard R E (eds). Clinical Oncology. Atlanta, Ga.: American Cancer Society; 1995:198-219).

Cancers are often the result of mutations that can occur in a large number of genes that play roles in a wide range of cellular processes. In many instances, cancer cells harbor mutations in genes that control processes such as cell growth, division, differentiation, or interaction with the extracellular environment. As an example, mutations that increase the activity of HER2, which is a cell surface receptor that promotes cell growth and division, are implicated in many cancers.

In many cases, tumors are either resistant to a particular cancer therapy, or are initially sensitive to a particular therapy but later become resistant. The development of resistance is often the consequence of mutations that alter the activity of a cell component (e.g., a mutation that renders a signaling molecule constitutively active) or result in the altered expression of a gene (e.g., a mutation that results in the increased expression of a cell signaling receptor such as HER2). In some instances, resistance coincides with or results from the occurrence of mutations that transform a cancer to a more aggressive (e.g., metastatic) form. Metastatic cancers are typically correlated with a worsened prognosis compared to non-metastatic cancers.

Cancers that are characterized by the overexpression of HER2 (referred to as HER2 positive cancers) are often correlated with poor prognosis and/or are resistant to many standard therapies. Accordingly, there is a need for new therapies that are effective for the treatment of cancers such as HER2 positive cancers and/or metastatic HER2 positive cancers. The present invention satisfies this need, and provides other advantages as well.

All references cited herein, including patent applications, patent publications, and scientific literature, are herein incorporated by reference in their entirety, as if each individual reference were specifically and individually indicated to be incorporated by reference.

### SUMMARY

Provided herein is a method for treating breast cancer in a subject comprising administering a therapeutically effective amount of tucatinib, or salt or solvate thereof, to the subject, wherein the subject is not concurrently receiving treatment with a therapeutically effective amount of a substrate of a multidrug and toxin extrusion (MATE) protein. In some embodiments, the subject has not received treatment with the substrate of the MATE protein within the past 7 days. In some embodiments, the subject has not received treatment with the substrate of the MATE protein within the past 3 months. In some embodiments, the subject has not received treatment with the substrate of the MATE protein within the past 12 months. In some embodiments, the subject has not previously received treatment with the substrate of the MATE protein. In some of any of the embodiments herein, the MATE protein is MATE1. In some of any of the embodiments herein, the MATE protein is MATE2K. In some of any of embodiments herein, the substrate of the MATE protein is selected from the group consisting of metformin, oxazolidinone, fexofenadine, tetraethylammonium (TEA), N-methylphenylpyridinium (MPP+), paraquat, agmatine, cimetidine, procainamide, pramipexole, atenolol, serotonin, quinidine, verapamil, cisplatin, oxaliplatin, and pyrimethamine.

Also provided herein is a method for treating breast cancer in a subject comprising administering a therapeutically effective amount of tucatinib, or salt or solvate thereof, to the subject, wherein the subject is not concurrently receiving treatment with a therapeutically effective amount of a substrate of an organic cation transporter (OCT). In some embodiments, the subject has not received treatment with the substrate of the OCT within the past 7 days. In some embodiments, the subject has not received treatment with the substrate of the OCT within the past 3 months. In some embodiments, the subject has not received treatment with the substrate of the OCT protein within the past 12 months. In some embodiments, the subject has not previously received treatment with the substrate of the OCT. In some of any of the embodiments herein, the OCT is OCT1. In some of any of the embodiments herein, the OCT is OCT2. In some of any of the embodiments herein, is selected from the group consisting of metformin, oxazolidinone, fexofenadine, tetraethylammonium (TEA), N-methylphenylpyridinium (MPP+), paraquat, agmatine, cimetidine, procainamide, pramipexole, atenolol, serotonin, quinidine, verapamil, cisplatin, oxaliplatin, and pyrimethamine.

Also provided herein is a method for treating breast cancer in a subject comprising administering a therapeutically effective amount of tucatinib, or salt or solvate thereof, to the subject, wherein the subject does not have impaired renal function. In some embodiments, the subject has not had impaired renal function within the past 12 months. In some of any of the embodiments herein, impaired renal function is determined based on the serum creatinine level in the subject. In some of any of the embodiments herein, impaired renal function is determined based on the serum creatinine level in the subject. In some embodiments, the subject is male and the subject has a serum creatinine level of less than 1.5 mg/dL or the subject is female and has a serum creatinine level of less than to 1.4 mg/dL. In some of any of the embodiments herein, impaired renal function is determined based on the subject having abnormal creatinine clearance. In some of any of the embodiments herein, impaired renal function is determined based on the glomerular filtration rate of the subject.

Also provided herein is a method for treating breast cancer in a subject comprising administering a therapeutically effective amount of tucatinib, or salt or solvate thereof, to the subject, wherein the subject is not concurrently receiving treatment with a therapeutically effective amount of a compound that modulates the activity of a cytochrome p450 protein. In some embodiments, the subject has not received treatment with the compound that modulates the activity of the cytochrome p450 protein within the past 7 days. In some embodiments, the subject has not received treatment with the compound that modulates the activity of the cytochrome p450 protein within the past 3 months. In some embodiments, the subject has not received treatment with the compound that modulates the activity of the cytochrome p450 protein within the past 12 months. In some embodiments, the subject has not previously received treatment with compound that modulates the activity of the cytochrome p450 protein. In some of any of the embodiments herein, the compound that modulates the activity of the cytochrome p450 protein is an inhibitor of the activity of the cytochrome p450 protein. In some of any of the embodiments herein, the compound that modulates the activity of the cytochrome p450 protein is a strong inhibitor of the activity of the cytochrome p450 protein. In some of any of the embodiments herein, the compound that modulates the activity of the cytochrome p450 protein is an inducer of the activity of the cytochrome p450 protein. In some of any of the embodiments herein, the compound that modulates the activity of the cytochrome p450 protein is a strong inducer of the activity of the cytochrome p450 protein.

Also provided herein is a method for treating breast cancer in a subject comprising administering a therapeutically effective amount of tucatinib, or salt or solvate thereof, to the subject, wherein the subject is not concurrently receiving treatment with a therapeutically effective amount of a substrate of a cytochrome p450 protein. In some embodiments, the subject has not received treatment with the substrate of the cytochrome p450 protein within the past 7 days. In some embodiments, the subject has not received treatment with the substrate of the cytochrome p450 protein within the past 3 months. In some embodiments, the subject has not received treatment with the substrate of the cytochrome p450 protein within the past 12 months. In some embodiments, the subject has not previously received treatment with the substrate of the cytochrome p450 protein. In some of any of the embodiments herein, the substrate of the cytochrome p450 protein is a sensitive CYP3A substrate.

Also provided herein is a method for treating breast cancer in a subject comprising administering a therapeutically effective amount of tucatinib, or salt or solvate thereof, to the subject, wherein the subject is not concurrently receiving treatment with a therapeutically effective amount of a substrate of P-glycoprotein (P-gp). In some embodiments, the subject has not received treatment with the substrate of P-gp within the past 7 days. In some embodiments, the subject has not received treatment with the substrate of P-gp within the past 3 months. In some embodiments, the subject has not received treatment with the substrate of P-gp within the past 12 months. In some embodiments, the subject has not previously received treatment with substrate of P-gp. In some of any of the embodiments herein, the substrate of P-gp is a substrate with a narrow therapeutic index.

In some of any of the embodiments herein, the tucatinib is administered to the subject at a dose of about 150 mg to about 650 mg. In some of any of the embodiments herein, the tucatinib is administered to the subject at a dose of about 300 mg. In some of any of the embodiments herein, the tucatinib is administered once or twice per day. In some of any of the embodiments herein, the tucatinib is administered to the subject at a dose of about 300 mg twice per day. In some of any of the embodiments herein, the tucatinib is administered to the subject orally.

In some of any of the embodiments herein, the breast cancer is a HER2 positive breast cancer. In some embodiments, the cancer is determined to be HER2 positive using in situ hybridization, fluorescence in situ hybridization, or immunohistochemistry. In some of any of the embodiments herein, the breast cancer is metastatic. In some embodiments, the breast cancer has metastasized to the brain. In some of any of the embodiments herein, the breast cancer is locally advanced. In some of any of the embodiments herein, the breast cancer is unresectable.

In some of any of the embodiments herein, the method further comprises administering one or more additional therapeutic agents to the subject to treat the breast cancer. In some embodiments, the one or more additional therapeutic agents is selected from the group consisting of capecitabine and an anti-HER2 antibody, such as trastuzumab. In some embodiments, the capecitabine is administered to the subject orally. In some of any of the embodiments herein, the capecitabine is administered to the subject twice per day. In some embodiments, the trastuzumab is administered subcutaneously or intravenously. In some embodiments the trastuzumab is administered once about every 3 weeks.

In some of any of the embodiments herein, the subject has been previously treated with one or more additional therapeutic agents for the breast cancer. In some embodiments, the one or more additional therapeutic agents is an anti-HER2 antibody or anti-HER2 antibody-drug conjugate. In some of any of the embodiments herein, the subject has not been treated with another therapeutic agent for the breast cancer within the past 12 months. In some of any of the embodiments herein, the subject has not previously been treated with another therapeutic agent for the breast cancer.

In some of any of the embodiments herein, treating the subject results in a tumor growth inhibition (TGI) index of at least about 85%, such as about 100%. In some of any of the embodiments herein, one or more therapeutic effects in the subject is improved after administration of tucatinib to the subject relative to a baseline. In some embodiments, the one or more therapeutic effects is selected from the group consisting of: size of a tumor derived from the breast cancer, objective response rate, duration of response, time to response, progression free survival and overall survival. In some of any of the embodiments herein, the subject is a human.

### BRIEF DESCRIPTION OF THE DRAWINGS

FIG. 1 shows the schedule of treatment and assessments of the Phase I clinical study described in Example 1.

FIG. 2 is a graph showing the mean (±SD) plasma metformin concentration versus time profile for subjects treated with metformin alone or metformin in combination with tucatinib.

FIG. 3 is a graph showing the mean (±SD) plasma iohexol concentrations versus time profile for subjects treated with metformin alone or metformin in combination with tucatinib.

FIG. 4 is a graph showing the mean (±SD) plasma tucatinib trough concentration versus time.

FIG. 5 shows the schedule of treatment and assessments of Part A of the Phase I clinical study described in Example 2.

FIG. 6 shows the schedule of treatment and assessments of Part B of the Phase I clinical study described in Example 2.

FIG. 7 shows the schedule of treatment and assessments of Part C of the Phase I clinical study described in Example 2.

FIG. 8 shows the schedule of treatment and assessments of Part D of the Phase I clinical study described in Example 2.

FIG. 9 shows the schedule of treatment and assessments of Part E of the Phase I clinical study described in Example 2.

### DETAILED DESCRIPTION

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure. For purposes of the present invention, the following terms are defined.

Units, prefixes, and symbols are denoted in their Systeme International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “or” as used herein should in general be construed non-exclusively. For example, a claim to “a composition comprising A or B” would typically present an aspect with a composition comprising both A and B. “Or” should, however, be construed to exclude those aspects presented that cannot be combined without contradiction (e.g., a composition pH that is between 9 and 10 or between 7 and 8).

The group “A or B” is typically equivalent to the group “selected from the group consisting of A and B.”

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that aspects and embodiments of the invention described herein include “comprising,” “consisting,” and “consisting essentially of” aspects and embodiments.

The terms “about” and “approximately” as used herein shall generally mean an acceptable degree of error for the quantity measured given the nature or precision of the measurements. Typical, exemplary degrees of error are within 20 percent (%), preferably within 10%, and more preferably within 5% of a given value or range of values. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written description support for a claim limitation of, e.g., “0.98X.” The terms “about” and “approximately,” particularly in reference to a given quantity, encompass and describe the given quantity itself.

Alternatively, in biological systems, the terms “about” and “approximately” may mean values that are within an order of magnitude, preferably within 5-fold, and more preferably within 2-fold of a given value. Numerical quantities given herein are approximate unless stated otherwise, meaning that the term “about” or “approximately” can be inferred when not expressly stated.

When “about” is applied to the beginning of a numerical range, it applies to both ends of the range. Thus, “from about 5 to 20%” is equivalent to “from about 5% to about 20%.” When “about” is applied to the first value of a set of values, it applies to all values in that set. Thus, “about 7, 9, or 11 mg/kg” is equivalent to “about 7, about 9, or about 11 mg/kg.”

The term “comprising” as used herein should in general be construed as not excluding additional ingredients. For example, a claim to “a composition comprising A” would cover compositions that include A and B; A, B, and C; A, B, C, and D; A, B, C, D, and E; and the like.

As used herein, the term “co-administering” includes sequential or simultaneous administration of two or more structurally different compounds. For example, two or more structurally different pharmaceutically active compounds can be co-administered by administering a pharmaceutical composition adapted for oral administration that contains two or more structurally different active pharmaceutically active compounds. As another example, two or more structurally different compounds can be co-administered by administering one compound and then administering the other compound. The two or more structurally different compounds can be comprised of an anti-HER2 antibody and tucatinib. In some instances, the co-administered compounds are administered by the same route. In other instances, the co-administered compounds are administered via different routes. For example, one compound can be administered orally, and the other compound can be administered, e.g., sequentially or simultaneously, via intravenous, intramuscular, subcutaneous, or intraperitoneal injection. The simultaneously or sequentially administered compounds or compositions can be administered such that an anti-HER2 antibody and tucatinib are simultaneously present in a subject or in a cell at an effective concentration.

A “cancer” refers to a broad group of various diseases characterized by the uncontrolled growth of abnormal cells in the body. A “cancer” or “cancer tissue” can include a tumor. Unregulated cell division and growth results in the formation of malignant tumors that invade neighboring tissues and can also metastasize to distant parts of the body through the lymphatic system or bloodstream. Following metastasis, the distal tumors can be said to be “derived from” the pre-metastasis tumor. For example, a “tumor derived from” a breast cancer refers to a tumor that is the result of a metastasized breast cancer.

In the context of cancer, the term “stage” refers to a classification of the extent of cancer. Factors that are considered when staging a cancer include but are not limited to tumor size, tumor invasion of nearby tissues, and whether the tumor has metastasized to other sites. The specific criteria and parameters for differentiating one stage from another can vary depending on the type of cancer. Cancer staging is used, for example, to assist in determining a prognosis or identifying the most appropriate treatment option(s).

One non-limiting example of a cancer staging system is referred to as the “TNM” system. In the TNM system, “T” refers to the size and extent of the main tumor, “N” refers to the number of nearby lymph nodes to which the cancer has spread, and “M” refers to whether the cancer has metastasized. “TX” denotes that the main tumor cannot be measured, “T0” denotes that the main tumor cannot be found, and “T1,” “T2,” “T3,” and “T4” denote the size or extent of the main tumor, wherein a larger number corresponds to a larger tumor or a tumor that has grown into nearby tissues. “NX” denotes that cancer in nearby lymph nodes cannot be measured, “NO” denotes that there is no cancer in nearby lymph nodes, and “N1,” “N2,” “N3,” and “N4” denote the number and location of lymph nodes to which the cancer has spread, wherein a larger number corresponds to a greater number of lymph nodes containing the cancer. “MX” denotes that metastasis cannot be measured, “MO” denotes that no metastasis has occurred, and “MI” denotes that the cancer has metastasized to other parts of the body.

As another non-limiting example of a cancer staging system, cancers are classified or graded as having one of five stages: “Stage 0,” “Stage I,” “Stage II,” “Stage III,” or “Stage IV.” Stage 0 denotes that abnormal cells are present, but have not spread to nearby tissue. This is also commonly called carcinoma in situ (CIS). CIS is not cancer, but may subsequently develop into cancer. Stages I, II, and III denote that cancer is present. Higher numbers correspond to larger tumor sizes or tumors that have spread to nearby tissues. Stage IV denotes that the cancer has metastasized. One of skill in the art will be familiar with the different cancer staging systems and readily be able to apply or interpret them.

The term “HER2” (also known as also known as HER2/neu, ERBB2, CD340, receptor tyrosine-protein kinase erbB-2, proto-oncogene Neu, and human epidermal growth factor receptor 2) refers to a member of the human epidermal growth factor receptor (HER/EGFR/ERBB) family of receptor tyrosine kinases. Amplification or overexpression of HER2 plays a significant role in the development and progression of certain aggressive types of cancer, including colorectal cancer, gastric cancer, lung cancer (e.g., non-small cell lung cancer (NSCLC)), biliary cancers (e.g., cholangiocarcinoma, gallbladder cancer), bladder cancer, esophageal cancer, melanoma, ovarian cancer, liver cancer, prostate cancer, pancreatic cancer, small intestine cancer, head and neck cancer, uterine cancer, cervical cancer, and breast cancer. Non-limiting examples of HER2 nucleotide sequences are set forth in GenBank reference numbers NP_001005862, NP_001289936, NP_001289937, NP 001289938, and NP_004448. Non-limiting examples of HER2 peptide sequences are set forth in GenBank reference numbers NP 001005862, NP_001276865, NP 001276866, NP_001276867, and NP_004439.

When HER2 is amplified or overexpressed in or on a cell, the cell is referred to as being “HER2 positive.” The level of HER2 amplification or overexpression in HER2 positive cells is commonly expressed as a score ranging from 0 to 3 (i.e., HER2 0, HER2 1+, HER2 2+, or HER2 3+), with higher scores corresponding to greater degrees of expression.

The term “tucatinib,” also known as ONT-380 and ARRY-380, refers to the small molecule tyrosine kinase inhibitor that suppresses or blocks HER2 activation. Tucatinib has the following structure:

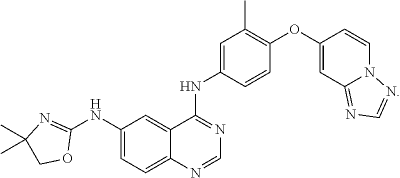 [SMILES: Cc1cc(Nc2ncnc3ccc(NC4=NC(C)(C)CO4)cc23)ccc1Oc1ccn2ncnc2c1]

The term “anti-HER2 antibody” refers to an antibody that binds to the HER2 protein. Anti-HER2 antibodies used for the treatment of cancer are typically monoclonal, although polyclonal antibodies are not excluded by the term. Anti-HER2 antibodies inhibit HER2 activation or downstream signaling by various mechanisms. As non-limiting examples, anti-HER2 antibodies can prevent ligand binding, receptor activation or receptor signal propagation, result in reduced HER2 expression or localization to the cell surface, inhibit HER2 cleavage, or induce antibody-mediated cytotoxicity. Non-limiting examples of anti-HER2 antibodies that are suitable for use in the methods and compositions of the present invention include trastuzumab, pertuzumab, ado-trastuzumab emtansine (also known as T-DM1), margetuximab, and combinations thereof.

The term “tumor growth inhibition (TGI) index” refers to a value used to represent the degree to which an agent (e.g., tucatinib, capecitabine, an anti-HER2 antibody, or a combination thereof) inhibits the growth of a tumor when compared to an untreated control. The TGI index is calculated for a particular time point (e.g., a specific number of days into an experiment or clinical trial) according to the following formula:

$$TGI = 1 - ( \frac{{Volume}_{treated ( TxDayX )} - {Volume}_{treated ( TxDay 0 )}}{{Volume}_{control ( TxDayX )} - {Volume}_{control ( TxDay 0 )}} ) \times 100 \% ,$$

where “Tx Day 0” denotes the first day that treatment is administered (i.e., the first day that an experimental therapy or a control therapy (e.g., vehicle only) is administered) and “Tx Day X” denotes X number of days after Day 0. Typically, mean volumes for treated and control groups are used. As a non-limiting example, in an experiment where study day 0 corresponds to “Tx Day 0” and the TGI index is calculated on study day 28 (i.e., “Tx Day 28”), if the mean tumor volume in both groups on study day 0 is 250 mm³ and the mean tumor volumes in the experimental and control groups are 125 mm³ and 750 mm³, respectively, then the TGI index on day 28 is 125%.

As used herein, the term “synergistic” or “synergy” refers to a result that is observed when administering a combination of components or agents (e.g., a combination of tucatinib and an anti-HER2 antibody) produces an effect (e.g., inhibition of tumor growth, prolongation of survival time) that is greater than the effect that would be expected based on the additive properties or effects of the individual components. In some embodiments, synergism is determined by performing a Bliss analysis (see, e.g., Foucquier et al. Pharmacol. Res. Perspect. (2015) 3(3):e00149; hereby incorporated by reference in its entirety for all purposes). The Bliss Independence model assumes that drug effects are outcomes of probabilistic processes, and asumes that the drugs act completely independently (i.e., the drugs do not interfere with one another (e.g., the drugs have different sites of action) but each contributes to a common result). According to the Bliss Independence model, the predicted effect of a combination of two drugs is calculated using the formula:

E_{AB}=E_{A}+E_{B}−E_{A}×E_{B},

where E_{A} and E_{B} represent the effects of drugs A and B, respectively, and E_{AB} represents the effect of a combination of drugs A and B. When the observed effect of the combination is greater than the predicted effect E_{AB}, then the combination of the two drugs is considered to be synergistic. When the observed effect of the combination is equal to E_{AB}, then the effect of the combination of the two drugs is considered to be additive. Alternatively, when the observed effect of the combination is less than E_{AB}, then the combination of the two drugs is considered to be antagonistic.

The observed effect of a combination of drugs can be based on, for example, the TGI index, tumor size (e.g., volume, mass), an absolute change in tumor size (e.g., volume, mass) between two or more time points (e.g., between the first day a treatment is adminstered and a particular number of days after treatment is first administered), the rate of change of tumor size (e.g., volume, mass) between two or more time points (e.g., between the first day a treatment is adminstered and a particular number of days after treatment is first administered), or the survival time of a subject or a population of subjects. When the TGI index is taken as a measure of the observed effect of a combination of drugs, the TGI index can be determined at one or more time points. When the TGI index is determined at two or more time points, in some instances the mean or median value of the multiple TGI indices can be used as a measure of the observed effect. Furthermore, the TGI index can be determined in a single subject or a population of subjects. When the TGI index is determined in a population, the mean or median TGI index in the population (e.g., at one or more time points) can be used as a measure of the observed effect. When tumor size or the rate of tumor growth is used as a measure of the observed effect, the tumor size or rate of tumor growth can be measured in a subject or a population of subjects. In some instances, the mean or median tumor size or rate of tumor growth is determined for a subject at two or more time points, or among a population of subjects at one or more time points. When survival time is measured in a population, the mean or median survival time can be used as a measure of the observed effect.

The predicted combination effect E_{AB} can be calculated using either a single dose or multiple doses of the drugs that make up the combination (e.g., tucatinib and an anti-HER2 antibody). In some embodiments, the predicted combination effect E_{AB} is calculated using only a single dose of each drug A and B (e.g., tucatinib and an anti-HER2 antibody), and the values E_{A} and E_{B} are based on the observed effect of each drug when administered as a single agent. When the values for E_{A} and E_{B} are based on the observed effects of administering drugs A and B as single agents, E_{A} and E_{B} can be based on, for example, TGI indices, tumor sizes (e.g., volume, mass) measured at one or more time points, absolute changes in tumor size (e.g., volume, mass) between two or more time points (e.g., between the first day a treatment is adminstered and a particular number of days after treatment is first administered), the rates of change of tumor sizes (e.g., volume, mass) between two or more time points (e.g., between the first day a treatment is adminstered and a particular number of days after treatment is first administered), or the survival time of a subject or a population of subjects in each treatment group.

When TGI indices are taken as a measure of the observed effects, the TGI indices can be determined at one or more time points. When TGI indices are determined at two or more time points, in some instances the mean or median values can be used as measures of the observed effects. Furthermore, the TGI indices can be determined in a single subject or a population of subjects in each treatment group. When the TGI indices are determined in populations of subjects, the mean or median TGI indices in each population (e.g., at one or more time points) can be used as measures of the observed effects. When tumor sizes or the rates of tumor growth are used as measures of the observed effects, the tumor sizes or rates of tumor growth can be measured in a subject or a population of subjects in each treatment group. In some instances, the mean or median tumor sizes or rates of tumor growth are determined for subjects at two or more time points, or among populations of subjects at one or more time points. When survival time is measured in a population, mean or median survival times can be used as measures of the observed effects.

In some embodiments, the predicted combination effect E_{AB} is calculated using a range of doses (i.e., the effects of each drug, when administered as a single agent, are observed at multiple doses and the observed effects at the multiple doses are used to determine the predicted combination effect at a specific dose). As a non-limiting example, E_{AB} can be calculated using values for E_{A} and E_{B} that are calculated according to the following formulae:

$${E}_{A} = {E}_{Amax} \times \frac{{a}^{p}}{{A}_{50}^{p} + {a}^{p}} \\ {E}_{B} = {E}_{Bmax} \times \frac{{b}^{q}}{{B}_{50}^{q} + {b}^{q}} ,$$

where E_{Amax} and E_{Bmax} are the maximum effects of drugs A and B, respectively, A₅₀ and B₅₀ are the half maximum effective doses of drugs A and B, respectively, a and b are administered doses of drugs A and B, respectively, and p and q are coefficients that are derived from the shapes of the dose-response curves for drugs A and B, respectively (see, e.g., Foucquier et al. Pharmacol. Res. Perspect. (2015) 3(3):e00149).

In some embodiments, a combination of two or more drugs is considered to be synergistic when the combination produces an observed TGI index that is greater than the predicted TGI index for the combination of drugs (e.g., when the predicted TGI index is based upon the assumption that the drugs produced a combined effect that is additive). In some instances, the combination is considered to be synergistic when the observed TGI index is at least about 1%, 2%, 3%, 4%, 5%, 6%, 7%, 8%, 9%, 10, 1%, 12%, 13%, 14%, 15%, 16%, 17%, 18%, 19%, 20%, 25%, 30%, 35%, 40%, 45%, 50%, 55% 60%, 65%, 70%, 75%, or 80% greater than the predicted TGI index for the combination of drugs.

In some embodiments, the rate of tumor growth (e.g., the rate of change of the size (e.g., volume, mass) of the tumor) is used to determine whether a combination of drugs is synergistic (e.g., the combination of drugs is synergistic when the rate of tumor growth is slower than would be expected if the combination of drugs produced an additive effect). In other embodiments, survival time is used to determine whether a combination of drugs is synergistic (e.g., a combination of drugs is synergistic when the survival time of a subject or population of subjects is longer than would be expected if the combination of drugs produced an additive effect).

“Treatment” or “therapy” of a subject refers to any type of intervention or process performed on, or the administration of an active agent to, the subject with the objective of reversing, alleviating, ameliorating, inhibiting, slowing down, or preventing the onset, progression, development, severity, or recurrence of a symptom, complication, condition, or biochemical indicia associated with a disease. In some embodiments, the disease is cancer.

A “subject” includes any human or non-human animal. The term “non-human animal” includes, but is not limited to, vertebrates such as non-human primates, sheep, dogs, and rodents such as mice, rats, and guinea pigs. In some embodiments, the subject is a human. The terms “subject” and “patient” and “individual” are used interchangeably herein.

An “effective amount” or “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

By way of example for the treatment of tumors, a therapeutically effective amount of an anti-cancer agent inhibits cell growth or tumor growth by at least about 10%, by at least about 20%, by at least about 30%, by at least about 40%, by at least about 50%, by at least about 60%, by at least about 70%, or by at least about 80%, by at least about 90%, by at least about 95%, by at least about 96%, by at least about 97%, by at least about 98%, or by at least about 99% in a treated subject(s) (e.g., one or more treated subjects) relative to an untreated subject(s) (e.g., one or more untreated subjects). In some embodiments, a therapeutically effective amount of an anti-cancer agent inhibits cell growth or tumor growth by 100% in a treated subject(s) (e.g., one or more treated subjects) relative to an untreated subject(s) (e.g., one or more untreated subjects).

In other embodiments of the disclosure, tumor regression can be observed and continue for a period of at least about 20 days, at least about 30 days, at least about 40 days, at least about 50 days, or at least about 60 days.

A therapeutically effective amount of a drug (e.g., tucatinib) includes a “prophylactically effective amount,” which is any amount of the drug that, when administered alone or in combination with an anti-cancer agent to a subject at risk of developing a cancer (e.g., a subject having a pre-malignant condition) or of suffering a recurrence of cancer, inhibits the development or recurrence of the cancer. In some embodiments, the prophylactically effective amount prevents the development or recurrence of the cancer entirely. “Inhibiting” the development or recurrence of a cancer means either lessening the likelihood of the cancer's development or recurrence, or preventing the development or recurrence of the cancer entirely.

As used herein, “subtherapeutic dose” means a dose of a therapeutic compound (e.g., tucatinib) that is lower than the usual or typical dose of the therapeutic compound when administered alone for the treatment of a hyperproliferative disease (e.g., cancer).

By way of example, an “anti-cancer agent” promotes cancer regression in a subject. In some embodiments, a therapeutically effective amount of the drug promotes cancer regression to the point of eliminating the cancer. “Promoting cancer regression” means that administering an effective amount of the drug, alone or in combination with an anti-cancer agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity or other adverse physiological effects at the cellular, organ and/or organism level (adverse effects) resulting from administration of the drug.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration that is at least the same as the treatment duration, or at least 1.5, 2.0, 2.5, or 3 times longer than the treatment duration.

As used herein, “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for PD, taking as reference the smallest SLD since the treatment started.

As used herein, “progression free survival” or “PFS” refers to the length of time during and after treatment during which the disease being treated (e.g., breast cancer) does not get worse. Progression-free survival may include the amount of time patients have experienced a complete response or a partial response, as well as the amount of time patients have experienced stable disease.

As used herein, “overall response rate” or “ORR” refers to the sum of complete response (CR) rate and partial response (PR) rate.

As used herein, “overall survival” or “OS” refers to the percentage of individuals in a group who are likely to be alive after a particular duration of time.

The term “weight-based dose”, as referred to herein, means that a dose administered to a subject is calculated based on the weight of the subject. For example, when a subject with 60 kg body weight requires 6.0 mg/kg of an agent, such as trasuzumab, one can calculate and use the appropriate amount of the agent (i.e., 360 mg) for administration to said subject.

The use of the term “fixed dose” with regard to a method of the disclosure means that two or more different agents (e.g., tucatinb and anti-HER2 antibody) are administered to a subject in particular (fixed) ratios with each other. In some embodiments, the fixed dose is based on the amount (e.g., mg) of the agents. In certain embodiments, the fixed dose is based on the concentration (e.g., mg/ml) of the agents. For example, a 1:2 ratio of tucatinib to an anti-HER2 antibody administered to a subject can mean about 300 mg of tucatinib and about 600 mg of the anti-HER2 antibody or about 3 mg/ml of tucatinib and about 6 mg/ml of the anti-HER2 antibody are administered to the subject.

The use of the term “flat dose” with regard to the methods and dosages of the disclosure means a dose that is administered to a subject without regard for the weight or body surface area (BSA) of the subject. The flat dose is therefore not provided as a mg/kg dose, but rather as an absolute amount of the agent (e.g., tucatinib or anti-HER2 antibody). For example, a subject with 60 kg body weight and a subject with 100 kg body weight would receive the same dose of tucatinb (e.g., 300 mg).

The phrase “pharmaceutically acceptable” indicates that the substance or composition must be compatible chemically and/or toxicologically, with the other ingredients comprising a formulation, and/or the mammal being treated therewith.

As used herein, the term “pharmaceutically acceptable carrier” refers to a substance that aids the administration of an active agent to a cell, an organism, or a subject. “Pharmaceutically acceptable carrier” refers to a carrier or excipient that can be included in the compositions of the invention and that causes no significant adverse toxicological effect on the subject. Non-limiting examples of pharmaceutically acceptable carriers include water, NaCl, normal saline solutions, lactated Ringer's, normal sucrose, normal glucose, binders, fillers, disintegrants, lubricants, coatings, sweeteners, flavors and colors, liposomes, dispersion media, microcapsules, cationic lipid carriers, isotonic and absorption delaying agents, and the like. The carrier may also be substances for providing the formulation with stability, sterility and isotonicity (e.g., antimicrobial preservatives, antioxidants, chelating agents and buffers), for preventing the action of microorganisms (e.g. antimicrobial and antifungal agents, such as parabens, chlorobutanol, phenol, sorbic acid and the like) or for providing the formulation with an edible flavor etc. In some instances, the carrier is an agent that facilitates the delivery of a small molecule drug or antibody to a target cell or tissue. One of skill in the art will recognize that other pharmaceutical carriers are useful in the present invention.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, pamoate (i.e., 4,4′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

“Administering” or “administration” refer to the physical introduction of a therapeutic agent to a subject, using any of the various methods and delivery systems known to those skilled in the art. Exemplary routes of administration include oral, intravenous, intramuscular, subcutaneous, intraperitoneal, spinal or other parenteral routes of administration, for example by injection or infusion (e.g., intravenous infusion). The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent can be administered via a non-parenteral route, or orally. Other non-parenteral routes include a topical, epidermal or mucosal route of administration, for example, intranasally, vaginally, rectally, sublingually or topically. Administration can also be performed, for example, once, a plurality of times, and/or over one or more extended periods.

The terms “baseline” or “baseline value” used interchangeably herein can refer to a measurement or characterization of a symptom before the administration of the therapy or at the beginning of administration of the therapy. The baseline value can be compared to a reference value in order to determine the reduction or improvement of a symptom of a disease contemplated herein (e.g., breast cancer). The terms “reference” or “reference value” used interchangeably herein can refer to a measurement or characterization of a symptom after administration of the therapy. The reference value can be measured one or more times during a dosage regimen or treatment cycle or at the completion of the dosage regimen or treatment cycle. A “reference value” can be an absolute value; a relative value; a value that has an upper and/or lower limit; a range of values; an average value; a median value: a mean value; or a value as compared to a baseline value.

Similarly, a “baseline value” can be an absolute value; a relative value; a value that has an upper and/or lower limit; a range of values; an average value; a median value; a mean value; or a value as compared to a reference value. The reference value and/or baseline value can be obtained from one individual, from two different individuals or from a group of individuals (e.g., a group of two, three, four, five or more individuals).

The term “monotherapy” as used herein means that the tucatinib, or salt or solvate thereof, is the only anti-cancer agent administered to the subject during the treatment cycle. Other therapeutic agents, however, can be administered to the subject. For example, anti-inflammatory agents or other agents administered to a subject with cancer to treat symptoms associated with cancer, but not the underlying cancer itself, including, for example inflammation, pain, weight loss, and general malaise, can be administered during the period of monotherapy.

An “adverse event” (AE) as used herein is any unfavorable and generally unintended or undesirable sign (including an abnormal laboratory finding), symptom, or disease associated with the use of a medical treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

A “serious adverse event” or “SAE” as used herein is an adverse event that meets one of the following criteria:
Is fatal or life-threatening (as used in the definition of a serious adverse event, “life-threatening” refers to an event in which the patient was at risk of death at the time of the event; it does not refer to an event which hypothetically might have caused death if it was more severe.
Results in persistent or significant disability/incapacity
Constitutes a congenital anomaly/birth defect
Is medically significant, i.e., defined as an event that jeopardizes the patient or may require medical or surgical intervention to prevent one of the outcomes listed above. Medical and scientific judgment must be exercised in deciding whether an AE is “medically significant”
Requires inpatient hospitalization or prolongation of existing hospitalization, excluding the following: 1) routine treatment or monitoring of the underlying disease, not associated with any deterioration in condition; 2) elective or pre-planned treatment for a pre-existing condition that is unrelated to the indication under study and has not worsened since signing the informed consent; and 3) social reasons and respite care in the absence of any deterioration in the patient's general condition.

The terms “once about every week,” “once about every two weeks,” or any other similar dosing interval terms as used herein mean approximate numbers. “Once about every week” can include every seven days±one day, i.e., every six days to every eight days. “Once about every two weeks” can include every fourteen days±two days, i.e., every twelve days to every sixteen days. “Once about every three weeks” can include every twenty-one days±three days, i.e., every eighteen days to every twenty-four days. Similar approximations apply, for example, to once about every four weeks, once about every five weeks, once about every six weeks, and once about every twelve weeks. In some embodiments, a dosing interval of once about every six weeks or once about every twelve weeks means that the first dose can be administered any day in the first week, and then the next dose can be administered any day in the sixth or twelfth week, respectively. In other embodiments, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

As described herein, any concentration range, percentage range, ratio range, or integer range is to be understood to include the value of any integer within the recited range and, when appropriate, fractions thereof (such as one tenth and one hundredth of an integer), unless otherwise indicated.

Various aspects of the disclosure are described in further detail in the following subsections.

### II. Description of the Embodiments

A. Methods for Treating Breast Cancer with Tucatinib

In one aspect, the present invention provides a method for treating breast cancer in a subject comprising administering a therapeutically effective amount of tucatinib, or salt or solvate thereof, to the subject, wherein the subject is not concurrently receiving treatment with a therapeutically effective amount of a substrate of a multidrug and toxin extrusion (MATE) protein. In some embodiments, the MATE protein is MATE1. In some embodiments, the MATE protein is MATE2K. When a subject is concurrently receiving treatment with a substrate of a MATE protein, it means that the subject received treatment with the substrate of the MATE protein within less than 7 days, such as within 1 day, within 2 days, within 3 days, within 4 days, within 5 days, or within 6 days, of being administered the therapeutically effective amount of tucatinib, or salt or solvate thereof.

In another aspect, the present invention provides a method for treating breast cancer in a subject comprising administering a therapeutically effective amount of tucatinib, or salt or solvate thereof, to the subject, wherein the subject has not received treatment with a therapeutically effective amount of a substrate of a MATE protein within a certain period of time of being administered the therapeutically effective amount of tucatinib, or salt or solvate thereof. In some embodiments, the subject has not received treatment with a therapeutically effective amount of a substrate of a MATE protein within the past 7 days, 10 days, 2 weeks, 3 weeks, 4 weeks, 6 weeks, 2 months, 3 months, 7 months, 8 months, 9 months, 10 months, 11 months, 12 months, 15 months, 18 months, 2 years, 3 years, 4 years, 5 years, 6 years, 7 years, 8 years, 9 years or 10 years prior to being administered the therapeutically effective amount of tucatinib, or salt or solvate thereof. In some embodiments, the subject has not received treatment with a therapeutically effective amount of a substrate of a MATE protein within the past 7 days. In some embodiments, the subject has not received treatment with a therapeutically effective amount of a substrate of a MATE protein within the past 3 months. In some embodiments, the subject has not received treatment with a therapeutically effective amount of a substrate of a MATE protein within the past 12 months. In some embodiments, the subject has not previously received treatment with the substrate of the MATE protein. In some embodiments, the MATE protein is MATE1. In some embodiments, the MATE protein is MATE2K.

In some embodiments, the substrate of the MATE protein is selected from the group consisting of metformin, oxazolidinone, fexofenadine, tetraethylammonium (TEA), N-methylphenylpyridinium (MPP+), paraquat, agmatine, cimetidine, procainamide, pramipexole, atenolol, serotonin, quinidine, verapamil, cisplatin, oxaliplatin, and pyrimethamine. In some embodiments, the substrate of the MATE protein is metformin.

In one aspect, the present invention provides a method for treating breast cancer in a subject comprising administering a therapeutically effective amount of tucatinib, or salt or solvate thereof, to the subject, wherein the subject is not concurrently receiving treatment with a therapeutically effective amount of a substrate of an organic cation transporter (OCT). In some embodiments, the OCT is OCT1. In some embodiments, the OCT protein is OCT2. When a subject is concurrently receiving treatment with a substrate of an OCT, it means that the subject received treatment with the substrate of the OCT within less than 7 days, such as within 1 day, within 2 days, within 3 days, within 4 days, within 5 days, or within 6 days, of being administered the therapeutically effective amount of tucatinib, or salt or solvate thereof.

In another aspect, the present invention provides a method for treating breast cancer in a subject comprising administering a therapeutically effective amount of tucatinib, or salt or solvate thereof, to the subject, wherein the subject has not received treatment with a therapeutically effective amount of a substrate of an OCT within a certain period of time of being administered the therapeutically effective amount of tucatinib, or salt or solvate thereof. In some embodiments, the subject has not received treatment with a therapeutically effective amount of a substrate of an OCT within the past 7 days, 10 days, 2 weeks, 3 weeks, 4 weeks, 6 weeks, 2 months, 3 months, 7 months, 8 months, 9 months, 10 months, 11 months, 12 months, 15 months, 18 months, 2 years, 3 years, 4 years, 5 years, 6 years, 7 years, 8 years, 9 years or 10 years prior to being administered the therapeutically effective amount of tucatinib, or salt or solvate thereof. In some embodiments, the subject has not received treatment with a therapeutically effective amount of a substrate of an OCT within the past 7 days. In some embodiments, the subject has not received treatment with a therapeutically effective amount of a substrate of an OCT within the past 3 months. In some embodiments, the subject has not received treatment with a therapeutically effective amount of a substrate of an OCT protein within the past 12 months. In some embodiments, the subject has not previously received treatment with the substrate of the OCT. In some embodiments, the OCT protein is OCT1. In some embodiments, the OCT protein is OCT2.

In some embodiments, the substrate of the OCT protein is selected from the group consisting of metformin, oxazolidinone, fexofenadine, tetraethylammonium (TEA), N-methylphenylpyridinium (MPP+), paraquat, agmatine, cimetidine, procainamide, pramipexole, atenolol, serotonin, quinidine, verapamil, cisplatin, oxaliplatin, and pyrimethamine. In some embodiments, the substrate of the OCT protein is metformin.

In another aspect, the present invention provides a method for treating breast cancer in a subject comprising administering a therapeutically effective amount of tucatinib, or salt or solvate thereof, to the subject, wherein the subject is not concurrently receiving treatment with a therapeutically effective amount of a substrate of an OCT or a substrate of a MATE protein. In some embodiments, the MATE protein is MATE1. In some embodiments, the MATE protein is MATE2K. In some embodiments, the substrate of the MATE protein is selected from the group consisting of metformin, oxazolidinone, fexofenadine, tetraethylammonium (TEA), N-methylphenylpyridinium (MPP+), paraquat, agmatine, cimetidine, procainamide, pramipexole, atenolol, serotonin, quinidine, verapamil, cisplatin, oxaliplatin, and pyrimethamine. In some embodiments, the substrate of the MATE protein is metformin. In some embodiments, the OCT is OCT1. In some embodiments, the OCT protein is OCT2. In some embodiments, the substrate of the OCT protein is selected from the group consisting of metformin, oxazolidinone, fexofenadine, tetraethylammonium (TEA), N-methylphenylpyridinium (MPP+), paraquat, agmatine, cimetidine, procainamide, pramipexole, atenolol, serotonin, quinidine, verapamil, cisplatin, oxaliplatin, and pyrimethamine. In some embodiments, the substrate of the OCT protein is metformin. When a subject is concurrently receiving treatment with a substrate of a MATE protein or a substrate of an OCT, it means that the subject received treatment with the substrate of the MATE protein or substrate of the OCT within less than 7 days, such as within 1 day, within 2 days, within 3 days, within 4 days, within 5 days, or within 6 days, of being administered the therapeutically effective amount of tucatinib, or salt or solvate thereof.

In another aspect, the present invention provides a method for treating breast cancer in a subject comprising administering a therapeutically effective amount of tucatinib, or salt or solvate thereof, to the subject, wherein the subject has not received treatment with a therapeutically effective amount of a substrate of a MATE protein or a substrate of an OCT within a certain period of time of being administered the therapeutically effective amount of tucatinib, or salt or solvate thereof. In some embodiments, the subject has not received treatment with a therapeutically effective amount of a substrate of a MATE protein within the past 7 days, 10 days, 2 weeks, 3 weeks, 4 weeks, 6 weeks, 2 months, 3 months, 7 months, 8 months, 9 months, 10 months, 11 months, 12 months, 15 months, 18 months, 2 years, 3 years, 4 years, 5 years, 6 years, 7 years, 8 years, 9 years or 10 years prior to being administered the therapeutically effective amount of tucatinib, or salt or solvate thereof. In some embodiments, the subject has not received treatment with a therapeutically effective amount of a substrate of a MATE protein within the past 7 days. In some embodiments, the subject has not received treatment with a therapeutically effective amount of a substrate of a MATE protein within the past 3 months. In some embodiments, the subject has not received treatment with a therapeutically effective amount of a substrate of a MATE protein within the past 12 months. In some embodiments, the subject has not previously received treatment with the substrate of the MATE protein. In some embodiments, the MATE protein is MATE1. In some embodiments, the MATE protein is MATE2K. In some embodiments, the substrate of the MATE protein is selected from the group consisting of metformin, oxazolidinone, fexofenadine, tetraethylammonium (TEA), N-methylphenylpyridinium (MPP+), paraquat, agmatine, cimetidine, procainamide, pramipexole, atenolol, serotonin, quinidine, verapamil, cisplatin, oxaliplatin, and pyrimethamine. In some embodiments, the substrate of the MATE protein is metformin. In some embodiments, the subject has not received treatment with a therapeutically effective amount of a substrate of an OCT within the past 1 day, 2 days, 3 days, 4 days, 5 days, 6 days, 7 days, 10 days, 2 weeks, 3 weeks, 4 weeks, 6 weeks, 2 months, 3 months, 7 months, 8 months, 9 months, 10 months, 11 months, 12 months, 15 months, 18 months, 2 years, 3 years, 4 years, 5 years, 6 years, 7 years, 8 years, 9 years or 10 years prior to being administered the therapeutically effective amount of tucatinib, or salt or solvate thereof. In some embodiments, the subject has not received treatment with a therapeutically effective amount of a substrate of an OCT within the past 7 days. In some embodiments, the subject has not received treatment with a therapeutically effective amount of a substrate of an OCT within the past 3 months. In some embodiments, the subject has not received treatment with a therapeutically effective amount of a substrate of an OCT protein within the past 12 months. In some embodiments, the subject has not previously received treatment with the substrate of the OCT. In some embodiments, the OCT protein is OCT1. In some embodiments, the OCT protein is OCT2. In some embodiments, the substrate of the OCT protein is selected from the group consisting of metformin, oxazolidinone, fexofenadine, tetraethylammonium (TEA), N-methylphenylpyridinium (MPP+), paraquat, agmatine, cimetidine, procainamide, pramipexole, atenolol, serotonin, quinidine, verapamil, cisplatin, oxaliplatin, and pyrimethamine. In some embodiments, the substrate of the OCT protein is metformin.

In another aspect, the present invention provides a method for treating breast cancer in a subject comprising administering a therapeutically effective amount of tucatinib, or salt or solvate thereof, to the subject, wherein the subject does not have impaired renal function. In another aspect, the present invention provides a method for treating breast cancer in a subject comprising administering a therapeutically effective amount of tucatinib, or salt or solvate thereof, to the subject, wherein the subject has not had impaired renal function within a certain period of time of being administered the therapeutically effective amount of tucatinib, or salt or solvate thereof. In some embodiments, the subject has not had impaired renal function within the past 1 day, 2 days, 3 days, 4 days, 5 days, 6 days, 7 days, 10 days, 2 weeks, 3 weeks, 4 weeks, 6 weeks, 2 months, 3 months, 7 months, 8 months, 9 months, 10 months, 11 months, 12 months, 15 months, 18 months, 2 years, 3 years, 4 years, 5 years, 6 years, 7 years, 8 years, 9 years or 10 years prior to being administered the therapeutically effective amount of tucatinib, or salt or solvate thereof. In some embodiments, the subject has not had impaired renal function within the past 12 months. In some embodiments, impaired renal function is determined based on the serum creatinine level in the subject. In some embodiments, a subject without impaired renal function is male and has a serum creatinine level of less than 1.5 mg/dL, less than 1.4 mg/dL, less than 1.3 mg/dL, less than 1.2 mg/dL, less than 1.1 mg/dL, less than 1.1 mg/dL or less than 1.0 mg/dL. In some embodiments, a subject without impaired renal function is male and has a serum creatinine level of less than 1.5 mg/dL. In some embodiments, a subject without impaired renal function is female and has a serum creatinine level of less than 1.4 mg/dL, less than 1.3 mg/dL, less than 1.2 mg/dL, less than 1.1 mg/dL, less than 1.1 mg/dL or less than 1.0 mg/dL. In some embodiments, a subject without impaired renal function is female and has a serum creatinine level of less than 1.4 mg/dL. In some embodiments, impaired renal function is determined based on the subject having abnormal creatinine clearance. In some embodiments, impaired renal function is determined based on the glomerular filtration rate of the subject.

In another aspect, the present invention provides a method for treating breast cancer in a subject comprising administering a therapeutically effective amount of tucatinib, or salt or solvate thereof, to the subject, wherein the subject is not concurrently receiving treatment with a therapeutically effective amount of a compound that modulates the activity of a cytochrome p450 protein. When a subject is concurrently receiving treatment with a compound that modulates the activity of a cytochrome p450 protein, it means that the subject received treatment with the compound that modulates the activity of the cytochrome p450 protein within less than 7 days, such as within 1 day, within 2 days, within 3 days, within 4 days, within 5 days, or within 6 days, of being administered the therapeutically effective amount of tucatinib, or salt or solvate thereof. In some embodiments, the compound that modulates the activity of the cytochrome p450 protein is an inhibitor of the activity of the cytochrome p450 protein. In some embodiments, the cytochrome p450 protein is CYP3A4. In some embodiments, the compound that inhibits the activity of CYP3A4 is selected from the group consisting of macrolide antibiotics (such as clarithromycin and troleandomycin), azole antibiotics (such as itraconazole, ketoconazole, voriconazole and posaconazole), nefazodone and diliazem. In some embodiments, the compound that inhibits the activity of CYP3A4 is a macrolide antibiotics (such as clarithromycin and troleandomycin). In some embodiments, the compound that inhibits the activity of CYP3A4 is clarithromycin. In some embodiments, the compound that inhibits the activity of CYP3A4 is troleandomycin. In some embodiments, the compound that inhibits the activity of CYP3A4 is an azole antibiotics (such as itraconazole, ketoconazole, voriconazole and posaconazole). In some embodiments, the compound that inhibits the activity of CYP3A4 is itraconazole. In some embodiments, the compound that inhibits the activity of CYP3A4 is ketoconazole. In some embodiments, the compound that inhibits the activity of CYP3A4 is voriconazole. In some embodiments, the compound that inhibits the activity of CYP3A4 is posaconazole. In some embodiments, the compound that inhibits the activity of CYP3A4 is nefazodone. In some embodiments, the compound that inhibits the activity of CYP3A4 is diliazem. In some embodiments, the cytochrome p450 protein is CYP2C8. In some embodiments, the compound that inhibits the activity of CYP2C8 is selected from the group consisting of gemfibrozil, montelukast, trimethoprim and clopidogrel. In some embodiments, the compound that inhibits the activity of CYP2C8 is gemfibrozil. In some embodiments, the compound that inhibits the activity of CYP2C8 In some embodiments, the compound that inhibits the activity of CYP2C8 is montelukast. In some embodiments, the compound that inhibits the activity of CYP2C8 is trimethoprim. In some embodiments, the compound that inhibits the activity of CYP2C8 is clopidogrel. In some embodiments, the compound that modulates the activity of the cytochrome p450 protein is an inducer of the activity of the cytochrome p450 protein. In some embodiments, the cytochrome p450 protein is CYP3A4. In some embodiments, the compound that induces the activity of CYP3A4 is selected from the group consisting of barbiturates, carbamazepine, phenytoin, rifampin and St. John's Wort. In some embodiments, the compound that induces the activity of CYP3A4 is a barbiturate. In some embodiments, the compound that induces the activity of CYP3A4 is carbamazepine. In some embodiments, the compound that induces the activity of CYP3A4 is phenytoin. In some embodiments, the compound that induces the activity of CYP3A4 is rifampin. In some embodiments, the compound that induces the activity of CYP3A4 is St. John's Wort. In some embodiments, the cytochrome p450 protein is CYP2C8. In some embodiments, the compound that induces the activity of CYP2C8 is rifampin.

In another aspect, the present invention provides a method for treating breast cancer in a subject comprising administering a therapeutically effective amount of tucatinib, or salt or solvate thereof, to the subject, wherein the subject has not received treatment with a therapeutically effective amount of a compound that modulates the activity of a cytochrome p450 within the past 7 days, 10 days, 2 weeks, 3 weeks, 4 weeks, 6 weeks, 2 months, 3 months, 7 months, 8 months, 9 months, 10 months, 11 months, 12 months, 15 months, 18 months, 2 years, 3 years, 4 years, 5 years, 6 years, 7 years, 8 years, 9 years or 10 years prior to being administered the therapeutically effective amount of tucatinib, or salt or solvate thereof. In some embodiments, the subject has not received treatment with a therapeutically effective amount of the compound that modulates the activity of the cytochrome p450 protein within the past 7 days. In some embodiments, the subject has not received treatment with a therapeutically effective amount of the compound that modulates the activity of the cytochrome p450 protein within the past 3 months. In some embodiments, the subject has not received treatment with a therapeutically effective amount of the compound that modulates the activity of the cytochrome p450 protein within the past 12 months. In some embodiments, the subject has not previously received treatment with the compound that modulates the activity of the cytochrome p450 protein. In some embodiments, the compound that modulates the activity of the cytochrome p450 protein is an inhibitor of the activity of the cytochrome p450 protein. In some embodiments, the compound that modulates the activity of the cytochrome p450 protein is a strong inhibitor of the activity of the cytochrome p450 protein. In some embodiments, the cytochrome p450 protein is CYP3A4. In some embodiments, the compound that inhibits the activity of CYP3A4 is selected from the group consisting of macrolide antibiotics (such as clarithromycin and troleandomycin), azole antibiotics (such as itraconazole, ketoconazole, voriconazole and posaconazole), nefazodone and diliazem. In some embodiments, the compound that inhibits the activity of CYP3A4 is a macrolide antibiotics (such as clarithromycin and troleandomycin). In some embodiments, the compound that inhibits the activity of CYP3A4 is clarithromycin. In some embodiments, the compound that inhibits the activity of CYP3A4 is troleandomycin. In some embodiments, the compound that inhibits the activity of CYP3A4 is an azole antibiotic (such as itraconazole, ketoconazole, voriconazole and posaconazole). In some embodiments, the compound that inhibits the activity of CYP3A4 is itraconazole. In some embodiments, the compound that inhibits the activity of CYP3A4 is ketoconazole. In some embodiments, the compound that inhibits the activity of CYP3A4 is voriconazole. In some embodiments, the compound that inhibits the activity of CYP3A4 is posaconazole. In some embodiments, the compound that inhibits the activity of CYP3A4 is nefazodone. In some embodiments, the compound that inhibits the activity of CYP3A4 is diliazem. In some embodiments, the cytochrome p450 protein is CYP2C8. In some embodiments, the compound that inhibits the activity of CYP2C8 is selected from the group consisting of gemfibrozil, montelukast, trimethoprim and clopidogrel. In some embodiments, the compound that inhibits the activity of CYP2C8 is gemfibrozil. In some embodiments, the compound that inhibits the activity of CYP2C8 In some embodiments, the compound that inhibits the activity of CYP2C8 is montelukast. In some embodiments, the compound that inhibits the activity of CYP2C8 is trimethoprim. In some embodiments, the compound that inhibits the activity of CYP2C8 is clopidogrel. In some embodiments, the compound that modulates the activity of the cytochrome p450 protein is an inducer of the activity of the cytochrome p450 protein. In some embodiments, the compound that modulates the activity of the cytochrome p450 protein is a strong inducer of the activity of the cytochrome p450 protein. In some embodiments, the cytochrome p450 protein is CYP3A4. In some embodiments, the compound that induces the activity of CYP3A4 is selected from the group consisting of barbiturates, carbamazepine, phenytoin, rifampin and St. John's Wort. In some embodiments, the compound that induces the activity of CYP3A4 is a barbiturate. In some embodiments, the compound that induces the activity of CYP3A4 is carbamazepine. In some embodiments, the compound that induces the activity of CYP3A4 is phenytoin. In some embodiments, the compound that induces the activity of CYP3A4 is rifampin. In some embodiments, the compound that induces the activity of CYP3A4 is St. John's Wort. In some embodiments, the cytochrome p450 protein is CYP2C8. In some embodiments, the compound that induces the activity of CYP2C8 is rifampin. In some embodiments, administering a therapeutically effective amount of tucatinib, or salt or solvate thereof, to a subject, concomitantly with a strong CYP3A/CYP2C8 inducer decreases tucatinib AUC which may reduce tucatinib efficacy. In some embodiments, administering a therapeutically effective amount of tucatinib, or salt or solvate thereof, to a subject, concomitantly with a strong CYP2C8 inhibitor increases tucatinib AUC which may increase the risk of toxicity.

In another aspect, the present invention provides a method for treating breast cancer in a subject comprising administering a therapeutically effective amount of tucatinib, or salt or solvate thereof, to the subject, wherein the subject has not received treatment with a therapeutically effective amount of a substrate of a cytochrome p450 protein within a certain period of time of being administered the therapeutically effective amount of tucatinib, or salt or solvate thereof. In some embodiments, the subject has not received treatment with a therapeutically effective amount of a substrate of a cytochrome p450 protein within the past 7 days, 10 days, 2 weeks, 3 weeks, 4 weeks, 6 weeks, 2 months, 3 months, 7 months, 8 months, 9 months, 10 months, 11 months, 12 months, 15 months, 18 months, 2 years, 3 years, 4 years, 5 years, 6 years, 7 years, 8 years, 9 years or 10 years prior to being administered the therapeutically effective amount of tucatinib, or salt or solvate thereof. In some embodiments, the subject has not received treatment with a therapeutically effective amount of a substrate of a cytochrome p450 protein within the past 7 days. In some embodiments, the subject has not received treatment with a therapeutically effective amount of a substrate of a cytochrome p450 protein within the past 3 months. In some embodiments, the subject has not received treatment with a therapeutically effective amount of a substrate of a cytochrome p450 protein within the past 12 months. In some embodiments, the subject has not previously received treatment with the substrate of the cytochrome p450 protein. In some embodiments, the cytochrome p450 protein is CYP3A4. In some embodiments, the cytochrome p450 protein is CYP2C8. In some embodiments, the substrate of the cytochrome p450 protein is a sensitive CYP3A substrate. In some embodiments, a sensitive CYP3A substrate refers to a drug whose plasma AUC value has been shown to increase 5-fold or higher when co-administered with a known CYP3A inhibitor. In some embodiments, the substrate of the cytochrome p450 protein is selected from the group consisting of budesonide, buspirone, eplerenone, eletriptan, felodipine, fluticasone, lovastatin, midazolam, saquinavir, sildenafil, simvastatin, triazolam, and vardenafil. In some embodiments, the substrate of the cytochrome p450 protein is budesonide. In some embodiments, the substrate of the cytochrome p450 protein is buspirone. In some embodiments, the substrate of the cytochrome p450 protein is eplerenon. In some embodiments, the substrate of the cytochrome p450 protein is eletriptan. In some embodiments, the substrate of the cytochrome p450 protein is felodipine. In some embodiments, the substrate of the cytochrome p450 protein is fluticasone. In some embodiments, the substrate of the cytochrome p450 protein is lovastatin. In some embodiments, the substrate of the cytochrome p450 protein is midazolam. In some embodiments, the substrate of the cytochrome p450 protein is saquinavir. In some embodiments, the substrate of the cytochrome p450 protein is sildenafil. In some embodiments, the substrate of the cytochrome p450 protein is simvastatin. In some embodiments, the substrate of the cytochrome p450 protein is triazolam. In some embodiments, the substrate of the cytochrome p450 protein is vardenafil. In some embodiments, administering a therapeutically effective amount of tucatinib, or salt or solvate thereof, to a subject, concomitantly with a CYP3A substrate may increase the plasma concentrations of the CYP3A substrate, which may lead to increased toxicity of the CYP3A substrate.

In another aspect, the present invention provides a method for treating breast cancer in a subject comprising administering a therapeutically effective amount of tucatinib, or salt or solvate thereof, to the subject, wherein the subject has not received treatment with a therapeutically effective amount of a substrate of P-glycoprotein (P-gp) within a certain period of time of being administered the therapeutically effective amount of tucatinib, or salt or solvate thereof. In some embodiments, the subject has not received treatment with a therapeutically effective amount of a substrate of P-gp within the past 7 days, 10 days, 2 weeks, 3 weeks, 4 weeks, 6 weeks, 2 months, 3 months, 7 months, 8 months, 9 months, 10 months, 11 months, 12 months, 15 months, 18 months, 2 years, 3 years, 4 years, 5 years, 6 years, 7 years, 8 years, 9 years or 10 years prior to being administered the therapeutically effective amount of tucatinib, or salt or solvate thereof. In some embodiments, the subject has not received treatment with a therapeutically effective amount of a substrate of P-gp within the past 7 days. In some embodiments, the subject has not received treatment with a therapeutically effective amount of a substrate of P-gp within the past 3 months. In some embodiments, the subject has not received treatment with a therapeutically effective amount of a substrate of P-gp within the past 12 months. In some embodiments, the subject has not previously received treatment with the substrate of P-gp. In some embodiments, the substrate of P-gp is a substrate with a narrow therapeutic index. In some embodiments, the substrate of P-gp is selected from the group consisting of amitriptyline, carbamazepine, clonidine, cyclosporine, digitoxin, digoxin, imipramine, phenobarbital, phenytoin, quinidine, rifampicin, sirolimus, tacrolimus, temsirolimus, trimipramine, vincristine, paclitaxel, and dabigatran etexilate. In some embodiments, the substrate of P-gp is amitriptyline. In some embodiments, the substrate of P-gp is carbamazepine. In some embodiments, the substrate of P-gp is clonidine. In some embodiments, the substrate of P-gp is cyclosporine. In some embodiments, the substrate of P-gp is digitoxin. In some embodiments, the substrate of P-gp is digoxin. In some embodiments, the substrate of P-gp is imipramine. In some embodiments, the substrate of P-gp is phenobarbital. In some embodiments, the substrate of P-gp is phenytoin. In some embodiments, the substrate of P-gp is quinidine. In some embodiments, the substrate of P-gp is rifampicin. In some embodiments, the substrate of P-gp is sirolimus. In some embodiments, the substrate of P-gp is tacrolimus. In some embodiments, the substrate of P-gp is temsirolimus. In some embodiments, the substrate of P-gp is trimipramine. In some embodiments, the substrate of P-gp is vincristine. In some embodiments, the substrate of P-gp is paclitaxel. In some embodiments, the substrate of P-gp is dabigatran etexilate. In some embodiments, administering a therapeutically effective amount of tucatinib, or salt or solvate thereof, to a subject, concomitantly with a P-gp substrate, such as digoxin, may increase the plasma concentrations of the P-gp substrate, which may lead to increased risk of adverse reactions.

B. Tucatinib Dose and Administration

In some embodiments, a dose of tucatinib is between about 0.1 mg and 10 mg per kg of the subject's body weight (e.g., about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 mg per kg of the subject's body weight). In other embodiments, a dose of tucatinib is between about 10 mg and 100 mg per kg of the subject's body weight (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 mg per kg of the subject's body weight). In some embodiments, a dose of tucatinib is at least about 100 mg to 500 mg per kg of the subject's body weight (e.g., at least about 100, 125, 150, 175, 200, 225, 250, 275, 300, 325, 350, 375, 400, 425, 450, 475, or 500 mg per kg of the subject's body weight). In particular embodiments, a dose of tucatinib is between about 1 mg and 50 mg per kg of the subject's body weight (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg per kg of the subject's body weight). In some instances, a dose of tucatinib is about 50 mg per kg of the subject's body weight.

In some embodiments, a dose of tucatinib comprises between about 1 mg and 100 mg (e.g. about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 mg) of tucatinib. In other embodiments, a dose of tucatinib comprises between about 100 mg and 1,000 mg (e.g., about 100, 105, 110, 115, 120, 125, 130, 135, 140, 145, 150, 155, 160, 165, 170, 175, 180, 185, 190, 195, 200, 205, 210, 215, 220, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1,000 mg) of tucatinib. In particular embodiments, a dose of tucatinib is about 300 mg (e.g., when administered twice per day).

In some embodiments, a dose of tucatinib comprises at least about 1,000 mg to 10,000 mg (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000 or more mg) of tucatinib.

In some embodiments, a dose of tucatinib, or salt or solvate thereof, contains a therapeutically effective amount of tucatinib, or salt or solvate thereof. In other embodiments, a dose of tucatinib, or salt or solvate thereof, contains less than a therapeutically effective amount of tucatinib, or salt or solvate thereof, (e.g., when multiple doses are given in order to achieve the desired clinical or therapeutic effect).

Tucatinib, or salt or solvate thereof, can be administered by any suitable route and mode. Suitable routes of administering antibodies and/or antibody-drug conjugate of the present invention are well known in the art and may be selected by those of ordinary skill in the art. In one embodiment, tucatinib administered parenterally. Parenteral administration refers to modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion. In some embodiments, the route of administration of tucatinib is intravenous injection or infusion. In some embodiments, the route of administration of tucatinib is intravenous infusion. In some embodiments, the route of administration of tucatinib is intravenous injection or infusion. In some embodiments, the tucatinib is intravenous infusion. In some embodiments, the route of administration of tucatinib is oral.

In one embodiment of the methods or uses or product for uses provided herein, tucatinib is administered to the subject daily, twice daily, three times daily or four times daily. In some embodiments, tucatinib is administered to the subject every other day, once about every week or once about every three weeks. In some embodiments, tucatinib is administered to the subject once per day. In some embodiments, tucatinib is administered to the subject twice per day. In some embodiments, tucatinib is administered to the subject at a dose of about 300 mg twice per day. In some embodiments, tucatinib is administered to the subject at a dose of 300 mg twice per day. In some embodiments, tucatinib is administered to the subject at a dose of about 600 mg once per day. In some embodiments, tucatinib is administered to the subject at a dose of 600 mg once per day. In some embodiments, tucatinib is administered to the subject twice per day on each day of a 21 day treatment cycle. In some embodiments, the tucatinib is administered to the subject orally.

C. Breast Cancer

The 2014 World Cancer Report from WHO (The World health organization) reports that breast cancer is the second most common cancer worldwide, accounting for just over 1 million new cases annually. It states that in 2000 about 400,000 women died from breast cancer, representing 1.6 percent of all female deaths. The proportion of breast cancer deaths was far higher in the rich countries (2 percent of all female deaths) than in economically poor regions (0.5 percent). Thus, breast cancer is strongly related to the Western lifestyle. As developing countries succeed in achieving lifestyles similar to Europe, North America, Australia, New Zealand and Japan, they will also encounter much higher cancer rates, particularly cancers of the breast. Recent data supports this prediction and show a 20% increase in breast cancer from 2008 to 2012. (Carter D. “New global survey shows an increasing cancer burden”. Am J Nurs. 2014 March; 114(3): 17).

In some aspects, the invention provides a method for treating breast cancer in a subject comprising administering a therapeutically effective amount of tucatinib, or salt or solvate thereof, as described herein. In some embodiments, the breast cancer is a HER2 positive breast cancer. In some embodiments, the cancer is determined to be HER2 positive using in situ hybridization, fluorescence in situ hybridization, or immunohistochemistry. In some embodiments, the breast cancer is metastatic. In some embodiments, the breast cancer has metastasized to the brain. In some embodiments, the breast cancer is locally advanced. In some embodiments, the breast cancer is unresectable. In some embodiments, the subject has been previously treated with one or more additional therapeutic agents for the breast cancer. In some embodiments, the subject has been previously treated with one or more additional therapeutic agents for the breast cancer and did not respond to the treatment. In some embodiments, the subject has been previously treated with one or more additional therapeutic agents for the breast cancer and relapsed after the treatment. In some embodiments, the subject has been previously treated with one or more additional therapeutic agents for the breast cancer and experienced disease progression during the treatment. In some embodiments, the one or more additional therapeutic agents is an anti-HER2 antibody or anti-HER2 antibody-drug conjugate. In some embodiments, the one or more additional therapeutic agents is an anti-HER2 antibody. In some embodiments, the one or more additional therapeutic agents is anti-HER2 antibody-drug conjugate. In some embodiments, the subject has been previously treated with trastuzumab, pertuzumab and/or T-DM1. In some embodiments, the subject has been previously treated with trastuzumab. In some embodiments, the subject has been previously treated with pertuzumab. In some embodiments, the subject has been previously treated with T-DM1. In some embodiments, the subject has been previously treated with trastuzumab and pertuzumab. In some embodiments, the subject has been previously treated with trastuzumab and T-DM1. In some embodiments, the subject has been previously treated with pertuzumab and T-DM1. In some embodiments, the subject has been previously treated with trastuzumab, pertuzumab and T-DM1. In some embodiments, the subject has not been previously treated with another therapeutic agent for the breast cancer within the past 1 day, 2 days, 3 days, 4 days, 5 days, 6 days, 7 days, 10 days, 2 weeks, 3 weeks, 4 weeks, 6 weeks, 2 months, 3 months, 7 months, 8 months, 9 months, 10 months, 11 months, 12 months, 15 months, 18 months, 2 years, 3 years, 4 years, 5 years, 6 years, 7 years, 8 years, 9 years or 10 years prior to being administered the therapeutically effective amount of tucatinib, or salt or solvate thereof. In some embodiments, the subject has not been previously treated with another therapeutic agent for the breast cancer within the past 12 months prior to being administered the therapeutically effective amount of tucatinib, or salt or solvate thereof. In some embodiments, the subject has not been previously treated with another therapeutic agent for the breast cancer. In some embodiments, the subject has not been previously treated with lapatinib, neratinib, afatinib, or capecitabine. In some embodiments, the subject has not been previously treated with lapatinib. In some embodiments, the subject has not been previously treated with neratinib. In some embodiments, the subject has not been previously treated with afatinib. In some embodiments, the subject has not been previously treated with capecitabine.

In some embodiments, ther HER2 status of a sample cell is determined. The determination can be made before treatment (i.e., administration of tucatinib) begins, during treatment, or after treatment has been completed. In some instances, determination of the HER2 status results in a decision to change therapy (e.g., adding an anti-HER2 antibody to the treatment regimen, discontinuing the use of tucatinib, discontinuing therapy altogether, or switching from another treatment method to a method of the present invention).

In some embodiments, the sample cell is determined to be overexpressing or not overexpressing HER2. In particular embodiments, the cell is determined to be HER2 3+, HER2 2+, HER2 1+, or HER2 0 (i.e., HER is not overexpressed).

In some embodiments, the sample cell is a cancer cell. In some instances, the sample cell is obtained from a subject who has cancer. The sample cell can be obtained as a biopsy specimen, by surgical resection, or as a fine needle aspirate (FNA). In some embodiments, the sample cell is a circulating tumor cell (CTC).

HER2 expression can be compared to a reference cell. In some embodiments, the reference cell is a non-cancer cell obtained from the same subject as the sample cell. In other embodiments, the reference cell is a non-cancer cell obtained from a different subject or a population of subjects. In some embodiments, measuring expression of HER2 comprises, for example, determining HER2 gene copy number or amplification, nucleic acid sequencing (e.g., sequencing of genomic DNA or cDNA), measuring mRNA expression, measuring protein abundance, or a combination thereof. HER2 testing methods include immunohistochemistry (IHC), in situ hybridization, fluorescence in situ hybridization (FISH), chromogenic in situ hybridization (CISH), ELISAs, and RNA quantification (e.g., of HER2 expression) using techniques such as RT-PCR and microarray analysis.

In some embodiments, the sample cell is determined to be HER2 positive when HER2 is expressed at a higher level in the sample cell compared to a reference cell. In some embodiments, the cell is determined to be HER2 positive when HER2 is overexpressed at least about 1.5-fold (e.g., about 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, or more) compared to a reference cell. In particular embodiments, the cell is determined to be HER2 positive when HER2 is overexpressed at least about 1.5-fold compared to the reference cell.

In some embodiments, the sample cell is determined to be HER2 positive when the FISH or CISH signal ratio is greater than 2. In other embodiments, the sample cell is determined to be HER2 positive when the HER2 gene copy number is greater than 6.

D. Combination Therapy

In some aspects, a method of treatment as described herein further comprises administering one or more additional therapeutic agents to the subject to treat the breast cancer. In some embodiments, the one or more additional therapeutic agents is selected from the group consisting of capecitabine and an anti-HER2 antibody. In some embodiments, the one or more additional therapeutic agents is capecitabine. In some embodiments, the one or more additional therapeutic agents is an anti-HER2 antibody. In some embodiments, the one or more additional therapeutic agents are capecitabine and an anti-HER2 antibody. In some embodiments, the anti-HER2 antibody is selected from the group consisting of trastuzumab, pertuzumab, ado-trastuzumab emtansine, margetuximab, and a combination thereof. In some instances, the anti-HER2 antibody is a combination of trastuzumab and pertuzumab. In some embodiments, the anti-HER2 antibody is trastuzumab. In some embodiments, the one or more additional therapeutic agents are capecitabine and trasuzumab.

In some embodiments, a method of treatment described herein further comprises administering capecitabine to the subject at a dose based on the body surface area of the subject. In some embodiments, capecitabine is administered to the subject at a dose of about 500 mg/m₂ to about 1500 mg/m². In some embodiments, capecitabine is administered to the subject at a dose of about 500 mg/m², about 550 mg/m², about 600 mg/m², about 650 mg/m², about 700 mg/m², about 750 mg/m², about 800 mg/m², about 850 mg/m², about 900 mg/m², about 950 mg/m², about 1000 mg/m², about 1050 mg/m², about 1100 mg/m², about 1150 mg/m², about 1200 mg/m², about 1250 mg/m², about 1300 mg/m², about 1350 mg/m², about 1400 mg/m², about 1450 mg/m², or about 1500 mg/m². In some embodiments, capecitabine is administered to the subject at a dose of 500 mg/m² to 1500 mg/m². In some embodiments, capecitabine is administered to the subject at a dose of 500 mg/m², 550 mg/m², 600 mg/m², 650 mg/m², 700 mg/m², 750 mg/m², 800 mg/m², 850 mg/m², 900 mg/m², 950 mg/m², 1000 mg/m², 1050 mg/m², 1100 mg/m², 1150 mg/m², 1200 mg/m², 1250 mg/m², 1300 mg/m², 1350 mg/m², 1400 mg/m², 1450 mg/m², or 1500 mg/m². In some embodiments, capecitabine is administered to the subject daily, twice daily, three times daily or four times daily. In some embodiments, capecitabine is administered to the subject every other day, once about every week or once about every three weeks. In some embodiments, capecitabine is administered to the subject once per day. In some embodiments, capecitabine is administered to the subject twice per day. In some embodiments, capecitabine is administered to the subject twice per day on days 1-14 of a 21 day treatment cycle. In some embodiments, capecitabine is administered to the subject at a dose of about 1000 mg/m² twice per day. In some embodiments, capecitabine is administered to the subject at a dose of 1000 mg/m² twice per day. In some embodiments, capecitabine is administered to the subject at a dose of about 1000 mg/m² twice per day on days 1-14 of a 21 day treatment cycle. In some embodiments, capecitabine is administered to the subject at a dose of 1000 mg/m² twice per day on days 1-14 of a 21 day treatment cycle. In some embodiments, the capecitabine is administered to the subject orally.

In some embodiments, a method of treatment described herein further comprises administering an anti-HER2 antibody to the subject. In some embodiments, a dose of the anti-HER2 antibody is between about 0.1 mg and 10 mg per kg of the subject's body weight (e.g., about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 mg per kg of the subject's body weight). In some embodiments, a dose of the anti-HER2 antibody is between about 4 mg and 10 mg per kg of the subject's body weight. In some embodiments, a dose of the anti-HER2 antibody is between 4 mg and 10 mg per kg of the subject's body weight. In some embodiments, a dose of the anti-HER2 antibody is about 6 mg per kg of the subject's body weight. In some embodiments, a dose of the anti-HER2 antibody is about 8 mg per kg of the subject's body weight. In some embodiments, a dose of the anti-HER2 antibody is about 8 mg per kg of the subject's body weight for the first dose of the anti-HER2 antibody administered to the subject followed by subsequent doses of about 6 mg per kg of the subject's body weight. In some embodiments, a dose of the anti-HER2 antibody is 6 mg per kg of the subject's body weight. In some embodiments, a dose of the anti-HER2 antibody is 8 mg per kg of the subject's body weight. In some embodiments, a dose of the anti-HER2 antibody is 8 mg per kg of the subject's body weight for the first dose of the anti-HER2 antibody administered to the subject followed by subsequent doses of 6 mg per kg of the subject's body weight. In other embodiments, a dose of the anti-HER2 antibody is between about 10 mg and 100 mg per kg of the subject's body weight (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 mg per kg of the subject's body weight). In some embodiments, a dose of the anti-HER2 antibody is at least about 100 mg to 500 mg per kg of the subject's body weight (e.g., at least about 100, 125, 150, 175, 200, 225, 250, 275, 300, 325, 350, 375, 400, 425, 450, 475, 500, or more mg per kg of the subject's body weight). In some instances, a dose of the anti-HER2 antibody is about 6 mg per kg of the subject's body weight. In other instances, a dose of the anti-HER2 antibody is about 8 mg per kg of the subject's body weight. In some other instances, a dose of the anti-HER2 antibody is about 20 mg per kg of the subject's body weight. In some embodiments, a dose of the anti-HER2 antibody comprises between about 1 mg and 100 mg (e.g. about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 mg) of the anti-HER2 antibody. In other embodiments, a dose of the anti-HER2 antibody comprises between about 100 mg and 1,000 mg (e.g., about 100, 105, 110, 115, 120, 125, 130, 135, 140, 145, 150, 155, 160, 165, 170, 175, 180, 185, 190, 195, 200, 205, 210, 215, 220, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1,000 mg) of the anti-HER2 antibody. In particular embodiments, a dose of the anti-HER2 antibody comprises between about 100 mg and 400 mg (e.g., about 100, 125, 150, 175, 200, 225, 250, 275, 300, 325, 350, 375, or 400 mg) of the anti-HER2 antibody. In some embodiments, a dose of the anti-HER2 antibody is between about 400 mg and 800 mg. In some embodiments, a dose of the anti-HER2 antibody is between 400 mg and 800 mg. In some embodiments, a dose of the anti-HER2 antibody is about 600 mg. In some embodiments, a dose of the anti-HER2 antibody is 600 mg. As a non-limiting example, when using a dose of 6 mg/kg, a dose for a 50 kg subject will be about 300 mg. As another non-limiting example, when using a dose of 8 mg/kg, a dose for a 50 kg subject will be about 400 mg. In some embodiments, a dose of the anti-HER2 antibody comprises at least about 1,000 mg to 10,000 mg (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000 or more mg) of the anti-HER2 antibody. In some embodiments, a dose of the anti-HER2 antibody contains a therapeutically effective amount of the anti-HER2 antibody. In other embodiments, a dose of the anti-HER2 antibody contains less than a therapeutically effective amount of the anti-HER2 antibody (e.g., when multiple doses are given in order to achieve the desired clinical or therapeutic effect). In some embodiments, the anti-HER2 antibody is administered to the subject once about every 1 to 4 weeks. In certain embodiments, an anti-HER2 antibody is administered once about every 1 week, once about every 2 weeks, once about every 3 weeks or once about every 4 weeks. In one embodiment, an anti-HER2 antibody is administered once about every 3 weeks. In some embodiments, the anti-HER2 antibody is administered to the subject once every 1 to 4 weeks. In certain embodiments, an anti-HER2 antibody is administered once every 1 week, once about every 2 weeks, once about every 3 weeks or once about every 4 weeks. In one embodiment, an anti-HER2 antibody is administered once every 3 weeks. In some embodiments, the anti-HER2 antibody is administered to the subject subcutaneously. In some embodiments, the anti-HER2 antibody is administered to the subject intravenously. In some embodiments, the anti-HER2 antibody is selected from the group consisting of trastuzumab, pertuzumab, ado-trastuzumab emtansine, margetuximab, and a combination thereof. In some instances, the anti-HER2 antibody is a combination of trastuzumab and pertuzumab. In some embodiments, the anti-HER2 antibody is trastuzumab. In some embodiments, the anti-HER2 antibody is administered at a dose of about 600 mg once about every 3 weeks and the anti-HER2 antibody is administered subcutaneously. In some embodiments, the anti-HER2 antibody is administered at a dose of 600 mg once every 3 weeks and the anti-HER2 antibody is administered subcutaneously. In some embodiments, the anti-HER2 antibody is trastuzumab and is administered at a dose of about 600 mg once about every 3 weeks and the trastuzumab is administered subcutaneously. In some embodiments, the anti-HER2 antibody is trastuzumab and is administered at a dose of 600 mg once every 3 weeks and the trastuzumab is administered subcutaneously. In some embodiments, the anti-HER2 antibody is administered at a dose of about 6 mg/kg once about every 3 weeks and the anti-HER2 antibody is administered intravenously. In some embodiments, the anti-HER2 antibody is administered at a dose of about 8 mg/kg once about every 3 weeks and the anti-HER2 antibody is administered intravenously. In some embodiments, the anti-HER2 antibody is administered once about every 3 weeks at a dose of about 8 mg/kg for the first dose of the anti-HER2 antibody administered to the subject followed by subsequent doses of about 6 mg/kg, wherein anti-HER2 antibody is administered intravenously. In some embodiments, the anti-HER2 antibody is administered at a dose of 6 mg/kg once every 3 weeks and the anti-HER2 antibody is administered intravenously. In some embodiments, the anti-HER2 antibody is administered at a dose of 8 mg/kg once every 3 weeks and the anti-HER2 antibody is administered intravenously. In some embodiments, the anti-HER2 antibody is administered once every 3 weeks at a dose of 8 mg/kg for the first dose of the anti-HER2 antibody administered to the subject followed by subsequent doses of 6 mg/kg, wherein anti-HER2 antibody is administered intravenously. In some embodiments, the anti-HER2 antibody is trastuzumab and is administered at a dose of about 6 mg/kg once about every 3 weeks and the trastuzumab is administered intravenously. In some embodiments, the anti-HER2 antibody is trastuzumab and is administered at a dose of about 8 mg/kg once about every 3 weeks and the trastuzumab is administered intravenously. In some embodiments, the anti-HER2 antibody is trastuzumab and is administered once about every 3 weeks at a dose of about 8 mg/kg for the first dose of the trastuzumab administered to the subject followed by subsequent doses of about 6 mg/kg, wherein the trastuzumab is administered intravenously. In some embodiments, the anti-HER2 antibody is trastuzumab and is administered at a dose of 6 mg/kg once every 3 weeks and the trastuzumab is administered intravenously. In some embodiments, the anti-HER2 antibody is trastuzumab and is administered at a dose of 8 mg/kg once every 3 weeks and the trastuzumab is administered intravenously. In some embodiments, the anti-HER2 antibody is trastuzumab and is administered once every 3 weeks at a dose of 8 mg/kg for the first dose of trastuzumab administered to the subject followed by subsequent doses of 6 mg/kg, wherein the trastuzumab is administered intravenously. In some embodiments, the anti-HER2 antibody is trastuzumab and is administered to the subject on a 21 day treatment cycle and is administered to the subject once per treatment cycle.

In some embodiments, a method of treatment described herein comprises administering to the subject tucatinib, capecitabine and trastuzumab. In some embodiments, the tucatinib, capecitabine and trastuzumab are administered to the subject on a 21 day treatment cycle. In some embodiments, tucatinib is administered to the subject at a dose of about 300 mg twice per day. In some embodiments, tucatinib is administered to the subject at a dose of 300 mg twice per day. In some embodiments, tucatinib is administered to the subject at a dose of about 600 mg once per day. In some embodiments, tucatinib is administered to the subject at a dose of 600 mg once per day. In some embodiments, tucatinib is administered to the subject twice per day on each day of a 21 day treatment cycle. In some embodiments, the tucatinib is administered to the subject orally. In some embodiments, capecitabine is administered to the subject twice per day. In some embodiments, capecitabine is administered to the subject twice per day on days 1-14 of a 21 day treatment cycle. In some embodiments, capecitabine is administered to the subject at a dose of about 1000 mg/m² twice per day. In some embodiments, capecitabine is administered to the subject at a dose of 1000 mg/m² twice per day. In some embodiments, capecitabine is administered to the subject at a dose of about 1000 mg/m² twice per day on days 1-14 of a 21 day treatment cycle. In some embodiments, capecitabine is administered to the subject at a dose of 1000 mg/m² twice per day on days 1-14 of a 21 day treatment cycle. In some embodiments, the capecitabine is administered to the subject orally. In some embodiments, the anti-HER2 antibody is administered at a dose of about 6 mg/kg once about every 3 weeks and the anti-HER2 antibody is administered intravenously. In some embodiments, the anti-HER2 antibody is administered at a dose of about 8 mg/kg once about every 3 weeks and the anti-HER2 antibody is administered intravenously. In some embodiments, the anti-HER2 antibody is administered once about every 3 weeks at a dose of about 8 mg/kg for the first dose of the anti-HER2 antibody administered to the subject followed by subsequent doses of about 6 mg/kg, wherein anti-HER2 antibody is administered intravenously. In some embodiments, the anti-HER2 antibody is administered at a dose of 6 mg/kg once every 3 weeks and the anti-HER2 antibody is administered intravenously. In some embodiments, the anti-HER2 antibody is administered at a dose of 8 mg/kg once every 3 weeks and the anti-HER2 antibody is administered intravenously. In some embodiments, the anti-HER2 antibody is administered once every 3 weeks at a dose of 8 mg/kg for the first dose of the anti-HER2 antibody administered to the subject followed by subsequent doses of 6 mg/kg, wherein anti-HER2 antibody is administered intravenously. In some embodiments, the anti-HER2 antibody is trastuzumab and is administered at a dose of about 6 mg/kg once about every 3 weeks and the trastuzumab is administered intravenously. In some embodiments, the anti-HER2 antibody is trastuzumab and is administered at a dose of about 8 mg/kg once about every 3 weeks and the trastuzumab is administered intravenously. In some embodiments, the anti-HER2 antibody is trastuzumab and is administered once about every 3 weeks at a dose of about 8 mg/kg for the first dose of the trastuzumab administered to the subject followed by subsequent doses of about 6 mg/kg, wherein the trastuzumab is administered intravenously. In some embodiments, the anti-HER2 antibody is trastuzumab and is administered at a dose of 6 mg/kg once every 3 weeks and the trastuzumab is administered intravenously. In some embodiments, the anti-HER2 antibody is trastuzumab and is administered at a dose of 8 mg/kg once every 3 weeks and the trastuzumab is administered intravenously. In some embodiments, the anti-HER2 antibody is trastuzumab and is administered once every 3 weeks at a dose of 8 mg/kg for the first dose of trastuzumab administered to the subject followed by subsequent doses of 6 mg/kg, wherein the trastuzumab is administered intravenously. In some embodiments, the anti-HER2 antibody is trastuzumab and is administered to the subject on a 21 day treatment cycle and is administered to the subject once per treatment cycle.

E. Treatment Outcome

In some embodiments, treating the subject comprises inhibiting breast cancer cell growth, inhibiting breast cancer cell proliferation, inhibiting breast cancer cell migration, inhibiting breast cancer cell invasion, decreasing or eliminating one or more signs or symptoms of breast cancer, reducing the size (e.g., volume) of a breast cancer tumor, reducing the number of breast cancer tumors, reducing the number of breast cancer cells, inducing breast cancer cell necrosis, pyroptosis, oncosis, apoptosis, autophagy, or other cell death, increasing survival time of the subject, or enhancing the therapeutic effects of another drug or therapy.

In some embodiments, treating the subject as described herein results in a tumor growth inhibition (TGI) index that is between about 10% and 70% (e.g., about 10%, 15%, 20%, 25%, 30%, 35%, 40%, 45%, 50%, 55%, 60%, 65%, or 70%). Preferably, treating the subject results in a TGI index that is at least about 70% (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%). More preferably, treating the subject results in a TGI index that is at least about 85% (e.g., about 85%, 86%, 87%, 88%, 89%, 90%, 91%, 92%, 93%, 94%, 95%, 96%, 97%, 98%, 99%, or 100%). Even more preferably, treating the subject results in a TGI index that is at least about 95% (e.g., about 95%, 96%, 97%, 98%, 99%, or 100%). Most preferably, treating the subject results in a TGI index that is about 100% or more (e.g., about 100%, 101%, 102%, 103%, 104%, 105%, 106%, 107%, 108%, 109%, 110%, 111%, 112%, 113%, 114%, 115%, 116%, 117%, 118%, 119%, 120%, 125%, 130%, 135%, 140%, 145%, 150%, or more).

In particular embodiments, treating the subject with tucatinib, capecitabine and trastuzumab results in a TGI index that is greater than the TGI index that is observed when tucatinib, capecitabine or trastuzumab is used alone. In some instances, treating the subject results in a TGI index that is greater than the TGI index that is observed when tucatinib is used alone. In other instances, treating the subject results in a TGI index that is greater than the TGI index that is observed when capecitabine is used alone. In other instances, treating the subject results in a TGI index that is greater than the TGI index that is observed when trastuzumab is used alone. In some embodiments, treating the subject results in a TGI index that is at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65, 70%, 75%, or 80% greater than the TGI index that is observed when tucatinib, capecitabine or trastuzumab is used alone.

In some embodiments, the combination of the tucatinib, capecitabine and trastuzumab is synergistic. In particular embodiments, with respect to the synergistic combination, treating the subject results in a TGI index that is greater than the TGI index that would be expected if the combination of tucatinib, capecitabine and trastuzumab produced an additive effect. In some instances, the TGI index observed when a combination of tucatinib, capecitabine and trastuzumab is administered is at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, or 80% greater than the TGI index that would be expected if the combination of tucatinib, capecitabine and trastuzumab produced an additive effect.

In one aspect, a method of treating cancer with tucatinib as described herein results in an improvement in one or more therapeutic effects in the subject after administration of tucatinib relative to a baseline. In some embodiments, the one or more therapeutic effects is the size of the tumor derived from the breast cancer, the objective response rate, the duration of response, the time to response, progression free survival, overall survival, or any combination thereof. In one embodiment, the one or more therapeutic effects is the size of the tumor derived from the breast cancer. In one embodiment, the one or more therapeutic effects is decreased tumor size. In one embodiment, the one or more therapeutic effects is stable disease. In one embodiment, the one or more therapeutic effects is partial response. In one embodiment, the one or more therapeutic effects is complete response. In one embodiment, the one or more therapeutic effects is the objective response rate. In one embodiment, the one or more therapeutic effects is the duration of response. In one embodiment, the one or more therapeutic effects is the time to response. In one embodiment, the one or more therapeutic effects is progression free survival. In one embodiment, the one or more therapeutic effects is overall survival. In one embodiment, the one or more therapeutic effects is cancer regression.

In one embodiment of the methods or uses or product for uses provided herein, response to treatment with tucatinib as described herein may include the following criteria (RECIST Criteria 1.1):

| | Category | Criteria |
|---|---|---|
| Based on | Complete | Disappearance of all target lesions. Any pathological |
| target lesions | Response (CR) | lymph nodes must have reduction in short axis to <10 |
| | | mm. |
| | Partial Response | ≥30% decrease in the sum of the longest diameter |
| | (PR) | (LD) of target lesions, taking as reference the baseline |
| | | sum of LDs. |
| | Stable Disease | Neither sufficient shrinkage to qualify for PR nor |
| | (SD) | sufficient increase to qualify for PD, taking as |
| | | reference the smallest sum of LDs while in trial. |
| | Progressive | ≥20% (and ≥5 mm) increase in the sum of the LDs of |
| | Disease (PD) | target lesions, taking as reference the smallest sum of |
| | | the target LDs recorded while in trial or the appearance |
| | | of one or more new lesions. |
| Based on non- | CR | Disappearance of all non-target lesions and |
| target lesions | | normalization of tumor marker level. All lymph nodes |
| | | must be non-pathological in size (<10 mm short axis). |
| | SD | Persistence of one or more non-target lesion(s) or/and |
| | | maintenance of tumor marker level above the normal |
| | | limits. |
| | PD | Appearance of one or more new lesions and/or |
| | | unequivocal progression of existing non-target lesions. |

In one embodiment of the methods or uses or product for uses provided herein, the effectiveness of treatment with tucatinib described herein is assessed by measuring the objective response rate. In some embodiments, the objective response rate is the proportion of patients with tumor size reduction of a predefined amount and for a minimum period of time. In some embodiments the objective response rate is based upon RECIST v1.1. In one embodiment, the objective response rate is at least about 20%, at least about 25%, at least about 30%, at least about 35%, at least about 40%, at least about 45%, at least about 50%, at least about 60%, at least about 70%, or at least about 80%. In one embodiment, the objective response rate is at least about 20%-80%. In one embodiment, the objective response rate is at least about 30%-80%. In one embodiment, the objective response rate is at least about 40%-80%. In one embodiment, the objective response rate is at least about 50%-80%. In one embodiment, the objective response rate is at least about 60%-80%. In one embodiment, the objective response rate is at least about 70%-80%. In one embodiment, the objective response rate is at least about 80%. In one embodiment, the objective response rate is at least about 85%. In one embodiment, the objective response rate is at least about 90%. In one embodiment, the objective response rate is at least about 95%. In one embodiment, the objective response rate is at least about 98%. In one embodiment, the objective response rate is at least about 99%. In one embodiment, the objective response rate is at least 20%, at least 25%, at least 30%, at least 35%, at least 40%, at least 45%, at least 50%, at least 60%, at least 70%, or at least 80%. In one embodiment, the objective response rate is at least 20%-80%. In one embodiment, the objective response rate is at least 30%-80%. In one embodiment, the objective response rate is at least 40%-80%. In one embodiment, the objective response rate is at least 50%-80%. In one embodiment, the objective response rate is at least 60%-80%. In one embodiment, the objective response rate is at least 70%-80%. In one embodiment, the objective response rate is at least 80%. In one embodiment, the objective response rate is at least 85%. In one embodiment, the objective response rate is at least 90%. In one embodiment, the objective response rate is at least 95%. In one embodiment, the objective response rate is at least 98%. In one embodiment, the objective response rate is at least 99%. In one embodiment, the objective response rate is 100%.

In one embodiment of the methods or uses or product for uses provided herein, response to treatment with tucatinib described herein is assessed by measuring the size of a tumor derived from the cancer (e.g., breast cancer). In one embodiment, the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of tucatinib. In one embodiment, the size of a tumor derived from the cancer is reduced by at least about 10%-80%. In one embodiment, the size of a tumor derived from the cancer is reduced by at least about 20%-80%. In one embodiment, the size of a tumor derived from the cancer is reduced by at least about 30%-80%. In one embodiment, the size of a tumor derived from the cancer is reduced by at least about 40%-80%. In one embodiment, the size of a tumor derived from the cancer is reduced by at least about 50%-80%. In one embodiment, the size of a tumor derived from the cancer is reduced by at least about 60%-80%. In one embodiment, the size of a tumor derived from the cancer is reduced by at least about 70%-80%. In one embodiment, the size of a tumor derived from the cancer is reduced by at least about 80%. In one embodiment, the size of a tumor derived from the cancer is reduced by at least about 85%. In one embodiment, the size of a tumor derived from the cancer is reduced by at least about 90%. In one embodiment, the size of a tumor derived from the cancer is reduced by at least about 95%. In one embodiment, the size of a tumor derived from the cancer is reduced by at least about 98%. In one embodiment, the size of a tumor derived from the cancer is reduced by at least about 99%. In one embodiment, the size of a tumor derived from the cancer is reduced by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of tucatinib. In one embodiment, the size of a tumor derived from the cancer is reduced by at least 10%-80%. In one embodiment, the size of a tumor derived from the cancer is reduced by at least 20%-80%. In one embodiment, the size of a tumor derived from the cancer is reduced by at least 30%-80%. In one embodiment, the size of a tumor derived from the cancer is reduced by at least 40%-80%. In one embodiment, the size of a tumor derived from the cancer is reduced by at least 50%-80%. In one embodiment, the size of a tumor derived from the cancer is reduced by at least 60%-80%. In one embodiment, the size of a tumor derived from the cancer is reduced by at least 70%-80%. In one embodiment, the size of a tumor derived from the cancer is reduced by at least 80%. In one embodiment, the size of a tumor derived from the cancer is reduced by at least 85%. In one embodiment, the size of a tumor derived from the cancer is reduced by at least 90%. In one embodiment, the size of a tumor derived from the cancer is reduced by at least 95%. In one embodiment, the size of a tumor derived from the cancer is reduced by at least 98%. In one embodiment, the size of a tumor derived from the cancer is reduced by at least 99%. In one embodiment, the size of a tumor derived from the cancer is reduced by 100%. In some embodiments, the size of a tumor derived from a breast cancer is measured by mammography, sonography or magnetic resonance imaging (MRI). See Gruber et. al., 2013, BMC Cancer. 13:328.

In one embodiment of the methods or uses or product for uses provided described herein, response to treatment with tucatinib described herein, promotes regression of a tumor derived from the cancer (e.g., breast cancer). In one embodiment, a tumor derived from the cancer regresses by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the tucatinib described herein. In one embodiment, a tumor derived from the cancer regresses by at least about 10% to about 80%. In one embodiment, a tumor derived from the cancer regresses by at least about 20% to about 80%. In one embodiment, a tumor derived from the cancer regresses by at least about 30% to about 80%. In one embodiment, a tumor derived from the cancer regresses by at least about 40% to about 80%. In one embodiment, a tumor derived from the cancer regresses by at least about 50% to about 80%. In one embodiment, a tumor derived from the cancer regresses by at least about 60% to about 80%. In one embodiment, a tumor derived from the cancer regresses by at least about 70% to about 80%. In one embodiment, a tumor derived from the cancer regresses by at least about 80%. In one embodiment, a tumor derived from the cancer regresses by at least about 85%. In one embodiment, a tumor derived from the cancer regresses by at least about 90%. In one embodiment, a tumor derived from the cancer regresses by at least about 95%. In one embodiment, a tumor derived from the cancer regresses by at least about 98%. In one embodiment, a tumor derived from the cancer regresses by at least about 99%. In one embodiment, a tumor derived from the cancer regresses by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of tucatinib described herein. In one embodiment, a tumor derived from the cancer regresses by at least 10% to 80%. In one embodiment, a tumor derived from the cancer regresses by at least 20% to 80%. In one embodiment, a tumor derived from the cancer regresses by at least 30% to 80%. In one embodiment, a tumor derived from the cancer regresses by at least 40% to 80%. In one embodiment, a tumor derived from the cancer regresses by at least 50% to 80%. In one embodiment, a tumor derived from the cancer regresses by at least 60% to 80%. In one embodiment, a tumor derived from the cancer regresses by at least 70% to 80%. In one embodiment, a tumor derived from the cancer regresses by at least 80%. In one embodiment, a tumor derived from the cancer regresses by at least 85%. In one embodiment, a tumor derived from the cancer regresses by at least 90%. In one embodiment, a tumor derived from the cancer regresses by at least 95%. In one embodiment, a tumor derived from the cancer regresses by at least 98%. In one embodiment, a tumor derived from the cancer regresses by at least 99%. In one embodiment, a tumor derived from the cancer regresses by 100%. In some embodiments, regression of a tumor is determined by mammography, sonography or magnetic resonance imaging (MRI). See Gruber et. al., 2013, BMC Cancer. 13:328.

In one embodiment of the methods or uses or product for uses described herein, response to treatment with tucatinib described herein is assessed by measuring the time of progression free survival after administration of tucatinib. In some embodiments, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of tucatinib. In some embodiments, the subject exhibits progression-free survival of at least about 6 months after administration of tucatinib. In some embodiments, the subject exhibits progression-free survival of at least about one year after administration of tucatinib. In some embodiments, the subject exhibits progression-free survival of at least about two years after administration of tucatinib. In some embodiments, the subject exhibits progression-free survival of at least about three years after administration of tucatinib. In some embodiments, the subject exhibits progression-free survival of at least about four years after administration of tucatinib. In some embodiments, the subject exhibits progression-free survival of at least about five years after administration of tucatinib. In some embodiments, the subject exhibits progression-free survival of at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of tucatinib. In some embodiments, the subject exhibits progression-free survival of at least 6 months after administration of tucatinib. In some embodiments, the subject exhibits progression-free survival of at least one year after administration of tucatinib. In some embodiments, the subject exhibits progression-free survival of at least two years after administration of tucatinib. In some embodiments, the subject exhibits progression-free survival of at least three years after administration of tucatinib. In some embodiments, the subject exhibits progression-free survival of at least four years after administration of tucatinib. In some embodiments, the subject exhibits progression-free survival of at least five years after administration of tucatinib.

In one embodiment of the methods or uses or product for uses described herein, response to treatment with tucatinib described herein is assessed by measuring the time of overall survival after administration of tucatinib. In some embodiments, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of tucatinib. In some embodiments, the subject exhibits overall survival of at least about 6 months after administration of tucatinib. In some embodiments, the subject exhibits overall survival of at least about one year after administration of tucatinib. In some embodiments, the subject exhibits overall survival of at least about two years after administration of tucatinib. In some embodiments, the subject exhibits overall survival of at least about three years after administration of tucatinib. In some embodiments, the subject exhibits overall survival of at least about four years after administration of tucatinib. In some embodiments, the subject exhibits overall survival of at least about five years after administration of tucatinib. In some embodiments, the subject exhibits overall survival of at least 1 month, at least 2 months, at least 3 months, at least 4 months, at least 5 months, at least 6 months, at least 7 months, at least 8 months, at least 9 months, at least 10 months, at least 11 months, at least about 12 months, at least eighteen months, at least two years, at least three years, at least four years, or at least five years after administration of tucatinib. In some embodiments, the subject exhibits overall survival of at least 6 months after administration of tucatinib. In some embodiments, the subject exhibits overall survival of at least one year after administration of tucatinib. In some embodiments, the subject exhibits overall survival of at least two years after administration of tucatinib. In some embodiments, the subject exhibits overall survival of at least three years after administration of tucatinib. In some embodiments, the subject exhibits overall survival of at least four years after administration of tucatinib. In some embodiments, the subject exhibits overall survival of at least five years after administration of tucatinib.

In one embodiment of the methods or uses or product for uses described herein, response to treatment with tucatinib described herein is assessed by measuring the duration of response to tucatinib after administration of tucatinib. In some embodiments, the duration of response to tucatinib is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of tucatinib. In some embodiments, the duration of response to tucatinib is at least about 6 months after administration of tucatinib. In some embodiments, the duration of response to tucatinib is at least about one year after administration of tucatinib. In some embodiments, the duration of response to tucatinib is at least about two years after administration of tucatinib. In some embodiments, the duration of response to tucatinib is at least about three years after administration of tucatinib. In some embodiments, the duration of response to tucatinib is at least about four years after administration of tucatinib. In some embodiments, the duration of response to tucatinib is at least about five years after administration of tucatinib. In some embodiments, the duration of response to tucatinib is at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of tucatinib. In some embodiments, the duration of response to tucatinib is at least 6 months after administration of tucatinib. In some embodiments, the duration of response to tucatinib is at least one year after administration of tucatinib. In some embodiments, the duration of response to tucatinib is at least two years after administration of tucatinib. In some embodiments, the duration of response to tucatinib is at least three years after administration of tucatinib. In some embodiments, the duration of response to tucatinib is at least four years after administration of tucatinib. In some embodiments, the duration of response to tucatinib is at least five years after administration of tucatinib.

F. Compositions

In another aspect, the present invention provides a pharmaceutical composition comprising tucatinib and a pharmaceutically acceptable carrier. In another aspect, the present invention provides a pharmaceutical composition comprising capecitabine and a pharmaceutically acceptable carrier. In another aspect, the present invention provides a pharmaceutical composition comprising an anti-HER2 antibody and a pharmaceutically acceptable carrier. In another aspect, the present invention provides a pharmaceutical composition comprising tucatinib, capecitabine, and a pharmaceutically acceptable carrier. In another aspect, the present invention provides a pharmaceutical composition comprising tucatinib, an anti-HER2 antibody, and a pharmaceutically acceptable carrier. In another aspect, the present invention provides a pharmaceutical composition comprising capecitabine, an anti-HER2 antibody, and a pharmaceutically acceptable carrier. In another aspect, the present invention provides a pharmaceutical composition comprising tucatinib, capecitabine, an anti-HER2 antibody, and a pharmaceutically acceptable carrier. In some embodiments, the anti-HER2 antibody is a member selected from the group consisting of trastuzumab, pertuzumab, ado-trastuzumab emtansine, margetuximab, and a combination thereof. In some instances, the anti-HER2 antibody is a combination of trastuzumab and pertuzumab. In some embodiments, the anti-HER2 antibody is trastuzumab.

In some embodiments, tucatinib is present at a concentration between about 0.1 nM and 10 nM (e.g., about 0.1, 0.2, 0.3, 0.4, 0.5 0.6, 0.7, 0.8, 0.9, 1.0, 1.5, 2, 2.5, 3, 3.5, 4, 4.5, 5, 5.5, 6, 6.5, 7, 7.5, 8, 8.5, 9, 9.5, or 10 nM). In other embodiments, tucatinib is present at a concentration between about 10 nM and 100 nM (e.g., about 10, 15, 20, 25, 30, 35, 40, 45, 50, 55, 60, 65, 70, 75, 80, 85, 90, 95, or 100 nM). In some other embodiments, tucatinib is present at a concentration between about 100 nM and 1,000 nM (e.g., about 100, 150, 200, 250, 300, 350, 400, 450, 500, 550, 600, 650, 700, 750, 800, 850, 900, 950, or 1,000 nM). In yet other embodiments, tucatinib is present at a concentration at least about 1,000 nM to 10,000 nM (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000, or more nM).

In some embodiments, the anti-HER2 antibody is present at a concentration between about 0.1 nM and 10 nM (e.g., about 0.1, 0.2, 0.3, 0.4, 0.5 0.6, 0.7, 0.8, 0.9, 1.0, 1.5, 2, 2.5, 3, 3.5, 4, 4.5, 5, 5.5, 6, 6.5, 7, 7.5, 8, 8.5, 9, 9.5, or 10 nM). In other embodiments, the anti-HER2 antibody is present at a concentration between about 10 nM and 100 nM (e.g., about 10, 15, 20, 25, 30, 35, 40, 45, 50, 55, 60, 65, 70, 75, 80, 85, 90, 95, or 100 nM). In some other embodiments, the anti-HER2 antibody is present at a concentration between about 100 nM and 1,000 nM (e.g., about 100, 150, 200, 250, 300, 350, 400, 450, 500, 550, 600, 650, 700, 750, 800, 850, 900, 950, or 1,000 nM). In yet other embodiments, the anti-HER2 antibody is present at a concentration of at least about 1,000 nM to 10,000 nM (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000, or more nM).

In some embodiments, capecitabine is present at a concentration between about 0.1 nM and 10 nM (e.g., about 0.1, 0.2, 0.3, 0.4, 0.5 0.6, 0.7, 0.8, 0.9, 1.0, 1.5, 2, 2.5, 3, 3.5, 4, 4.5, 5, 5.5, 6, 6.5, 7, 7.5, 8, 8.5, 9, 9.5, or 10 nM). In other embodiments, capecitabine is present at a concentration between about 10 nM and 100 nM (e.g., about 10, 15, 20, 25, 30, 35, 40, 45, 50, 55, 60, 65, 70, 75, 80, 85, 90, 95, or 100 nM). In some other embodiments, capecitabine is present at a concentration between about 100 nM and 1,000 nM (e.g., about 100, 150, 200, 250, 300, 350, 400, 450, 500, 550, 600, 650, 700, 750, 800, 850, 900, 950, or 1,000 nM). In yet other embodiments, capecitabine is present at a concentration of at least about 1,000 nM to 10,000 nM (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000, or more nM).

The pharmaceutical compositions of the present invention may be prepared by any of the methods well-known in the art of pharmacy. Pharmaceutically acceptable carriers suitable for use with the present invention include any of the standard pharmaceutical carriers, buffers and excipients, including phosphate-buffered saline solution, water, and emulsions (such as an oil/water or water/oil emulsion), and various types of wetting agents or adjuvants. Suitable pharmaceutical carriers and their formulations are described in Remington's Pharmaceutical Sciences (Mack Publishing Co., Easton, 19th ed. 1995). Preferred pharmaceutical carriers depend upon the intended mode of administration of the active agent.

The pharmaceutical compositions of the present invention can include a combination of drugs (e.g., tucatinib, capecitabine, and an anti-HER2 antibody), or any pharmaceutically acceptable salts thereof, as active ingredients and a pharmaceutically acceptable carrier or excipient or diluent. A pharmaceutical composition may optionally contain other therapeutic ingredients.

The compositions (e.g., comprising tucatinib, capecitabine, an anti-HER2 antibody, or a combination thereof) can be combined as the active ingredients in intimate admixture with a suitable pharmaceutical carrier or excipient according to conventional pharmaceutical compounding techniques. Any carrier or excipient suitable for the form of preparation desired for administration is contemplated for use with the compounds disclosed herein.

The pharmaceutical compositions include those suitable for oral, topical, parenteral, pulmonary, nasal, or rectal administration. The most suitable route of administration in any given case will depend in part on the nature and severity of the cancer condition and also optionally the HER2 status or stage of the cancer.

Other pharmaceutical compositions include those suitable for systemic (e.g., enteral or parenteral) administration. Systemic administration includes oral, rectal, sublingual, or sublabial administration. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In particular embodiments, pharmaceutical compositions of the present invention may be administered intratumorally.

Compositions for pulmonary administration include, but are not limited to, dry powder compositions consisting of the powder of a compound described herein (e.g., tucatinib, capecitabine, an anti-HER2 antibody, or a combination thereof), or a salt thereof, and the powder of a suitable carrier or lubricant. The compositions for pulmonary administration can be inhaled from any suitable dry powder inhaler device known to a person skilled in the art.

Compositions for systemic administration include, but are not limited to, dry powder compositions consisting of the composition as set forth herein (e.g., tucatinib, capecitabine, an anti-HER2 anibody, or a combination thereof) and the powder of a suitable carrier or excipient. The compositions for systemic administration can be represented by, but not limited to, tablets, capsules, pills, syrups, solutions, and suspensions.

In some embodiments, the compositions (e.g., tucatinib, capecitabine, an anti-HER2 anibody, or a combination thereof) further include a pharmaceutical surfactant. In other embodiments, the compositions further include a cryoprotectant. In some embodiments, the cryoprotectant is selected from the group consisting of glucose, sucrose, trehalose, lactose, sodium glutamate, PVP, HPPCD, CD, glycerol, maltose, mannitol, and saccharose.

Pharmaceutical compositions or medicaments for use in the present invention can be formulated by standard techniques using one or more physiologically acceptable carriers or excipients. Suitable pharmaceutical carriers are described herein and in Remington: The Science and Practice of Pharmacy, 21st Ed., University of the Sciences in Philadelphia, Lippencott Williams & Wilkins (2005).

Controlled-release parenteral formulations of the compositions (e.g., tucatinib, capecitabine, an anti-HER2 anibody, or a combination thereof) can be made as implants, oily injections, or as particulate systems. For a broad overview of delivery systems see Banga, A. J., THERAPEUTIC PEPTIDES AND PROTEINS: FORMULATION, PROCESSING, AND DELIVERY SYSTEMS, Technomic Publishing Company, Inc., Lancaster, Pa., (1995), which is incorporated herein by reference. Particulate systems include microspheres, microparticles, microcapsules, nanocapsules, nanospheres, and nanoparticles.

Polymers can be used for ion-controlled release of compositions of the present invention. Various degradable and nondegradable polymeric matrices for use in controlled drug delivery are known in the art (Langer R., Accounts Chem. Res., 26:537-542 (1993)). For example, the block copolymer, polaxamer 407 exists as a viscous yet mobile liquid at low temperatures but forms a semisolid gel at body temperature. It has been shown to be an effective vehicle for formulation and sustained delivery of recombinant interleukin 2 and urease (Johnston et al., Pharm. Res., 9:425-434 (1992); and Pec et al., J. Parent. Sci. Tech., 44(2):58 65 (1990)). Alternatively, hydroxyapatite has been used as a microcarrier for controlled release of proteins (Ijntema et al., Int. J. Pharm., 112:215-224 (1994)). In yet another aspect, liposomes are used for controlled release as well as drug targeting of the lipid-capsulated drug (Betageri et al., LIPOSOME DRUG DELIVERY SYSTEMS, Technomic Publishing Co., Inc., Lancaster, Pa. (1993)). Numerous additional systems for controlled delivery of therapeutic proteins are known. See, e.g., U.S. Pat. Nos. 5,055,303, 5,188,837, 4,235,871, 4,501,728, 4,837,028 4,957,735 and 5,019,369, 5,055,303; 5,514,670; 5,413,797; 5,268,164; 5,004,697; 4,902,505; 5,506,206, 5,271,961; 5,254,342 and 5,534,496, each of which is incorporated herein by reference.

For oral administration of a combination of tucatinib, capecitabine, and/or an anti-HER2 anibody, a pharmaceutical composition or a medicament can take the form of, for example, a tablet or a capsule prepared by conventional means with a pharmaceutically acceptable excipient. The present invention provides tablets and gelatin capsules comprising tucatinib, capecitabine, an anti-HER2 anibody, or a combination thereof, or a dried solid powder of these drugs, together with (a) diluents or fillers, e.g., lactose, dextrose, sucrose, mannitol, sorbitol, cellulose (e.g., ethyl cellulose, microcrystalline cellulose), glycine, pectin, polyacrylates or calcium hydrogen phosphate, calcium sulfate, (b) lubricants, e.g., silica, talcum, stearic acid, magnesium or calcium salt, metallic stearates, colloidal silicon dioxide, hydrogenated vegetable oil, corn starch, sodium benzoate, sodium acetate or polyethyleneglycol; for tablets also (c) binders, e.g., magnesium aluminum silicate, starch paste, gelatin, tragacanth, methylcellulose, sodium carboxymethylcellulose, polyvinylpyrrolidone or hydroxypropyl methylcellulose; if desired (d) disintegrants, e.g., starches (e.g., potato starch or sodium starch), glycolate, agar, alginic acid or its sodium salt, or effervescent mixtures; (e) wetting agents, e.g., sodium lauryl sulphate, or (f) absorbents, colorants, flavors and sweeteners.

Tablets may be either film coated or enteric coated according to methods known in the art. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, or sweetening agents as appropriate. If desired, preparations for oral administration can be suitably formulated to give controlled release of the active compound(s).

Typical formulations for topical administration of tucatinib, capecitabine, an anti-HER2 anibody, or a combination thereof include creams, ointments, sprays, lotions, and patches. The pharmaceutical composition can, however, be formulated for any type of administration, e.g., intradermal, subdermal, intravenous, intramuscular, subcutaneous, intranasal, intracerebral, intratracheal, intraarterial, intraperitoneal, intravesical, intrapleural, intracoronary or intratumoral injection, with a syringe or other devices. Formulation for administration by inhalation (e.g., aerosol), or for oral or rectal administration is also contemplated.

Suitable formulations for transdermal application include an effective amount of one or more compounds described herein, optionally with a carrier. Preferred carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used.

The compositions and formulations set forth herein (e.g., tucatinib, capecitabine, an anti-HER2 anibody, or a combination thereof) can be formulated for parenteral administration by injection, for example by bolus injection or continuous infusion. Formulations for injection can be presented in unit dosage form, for example, in ampules or in multi-dose containers, with an added preservative. Injectable compositions are preferably aqueous isotonic solutions or suspensions, and suppositories are preferably prepared from fatty emulsions or suspensions. The compositions may be sterilized or contain adjuvants, such as preserving, stabilizing, wetting or emulsifying agents, solution promoters, salts for regulating the osmotic pressure or buffers. Alternatively, the active ingredient(s) can be in powder form for constitution with a suitable vehicle, for example, sterile pyrogen-free water, before use. In addition, they may also contain other therapeutically valuable substances. The compositions are prepared according to conventional mixing, granulating or coating methods, respectively.

For administration by inhalation, the compositions (e.g., comprising tucatinib, capecitabine, an anti-HER2 anibody, or a combination thereof) may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound(s) and a suitable powder base, for example, lactose or starch.

The compositions (e.g., comprising tucatinib, capecitabine, an anti-HER2 anibody, or a combination thereof) can also be formulated in rectal compositions, for example, suppositories or retention enemas, for example, containing conventional suppository bases, for example, cocoa butter or other glycerides.

Furthermore, the active ingredient(s) can be formulated as a depot preparation. Such long-acting formulations can be administered by implantation (for example, subcutaneously or intramuscularly) or by intramuscular injection. Thus, for example, one or more of the compounds described herein can be formulated with suitable polymeric or hydrophobic materials (for example as an emulsion in an acceptable oil) or ion exchange resins, or as sparingly soluble derivatives, for example, as a sparingly soluble salt.

G. Articles of Manufacture and Kits

In another aspect, the present invention provides an article of manufacture or kit for treating or ameliorating the effects of breast cancer in a subject, the article of manufacture or kit comprising a pharmaceutical composition of the present invention (e.g., a pharmaceutical composition comprising tucatinib, capecitabine, an anti-HER2 antibody, or a combination thereof). In some embodiments, the anti-HER2 antibody is trastuzumab, pertuzumab, ado-trastuzumab emtansine, margetuximab, or a combination thereof. In some instances, the anti-HER2 antibody is a combination of trastuzumab and pertuzumab. In some embodiments, the anti-HER2 antibody is trastuzumab.

The articles of manufacture or kits are suitable for treating or ameliorating the effects of breast cancers, particularly HER2 positive and/or metastatic breast cancers. In some embodiments, the cancer is an advanced cancer. In some other embodiments, the cancer is a drug-resistant cancer. In some instances, the cancer is a multidrug-resistant cancer.

Materials and reagents to carry out the various methods of the present invention can be provided in articles of manufacture or kits to facilitate execution of the methods. As used herein, the term “kit” includes a combination of articles that facilitates a process, assay, analysis, or manipulation. In particular, kits of the present invention find utility in a wide range of applications including, for example, diagnostics, prognostics, therapy, and the like.

Articles of manufacture or kits can contain chemical reagents as well as other components. In addition, the articles of manufacture or kits of the present invention can include, without limitation, instructions to the user, apparatus and reagents for administering combinations of tucatinib, capecitabine and anti-HER2 antibodies or pharmaceutical compositions thereof, sample tubes, holders, trays, racks, dishes, plates, solutions, buffers, or other chemical reagents. In some embodiments, the articles of manufacture or kits contain instructions, apparatus, or reagents for determining the genotype of a gene (e.g., KRAS, NRAS, BRAF) or determining the expression of HER2 in a sample. Articles of manufacture or kits of the present invention can also be packaged for convenient storage and safe shipping, for example, in a box having a lid.

### III. Exemplary Embodiments

Among the embodiments provided herein are:

1. A method for treating breast cancer in a subject comprising administering a therapeutically effective amount of tucatinib, or salt or solvate thereof, to the subject, wherein the subject is not concurrently receiving treatment with a therapeutically effective amount of a substrate of a multidrug and toxin extrusion (MATE) protein.
2. The method of embodiment 1, wherein the subject has not received treatment with the substrate of the MATE protein within the past 7 days.
3. The method of embodiment 1, wherein the subject has not received treatment with the substrate of the MATE protein within the past 3 months.
4. The method of embodiment 1, wherein the subject has not received treatment with the substrate of the MATE protein within the past 12 months.
5. The method of embodiment 1, wherein the subject has not previously received treatment with the substrate of the MATE protein.
6. The method of any one of embodiments 1-5, wherein the MATE protein is MATE1.
7. The method of any one of embodiments 1-5, wherein the MATE protein is MATE2K.
8. The method of anyone of embodiments 1-7, wherein the substrate of the MATE protein is selected from the group consisting of metformin, oxazolidinone, fexofenadine, tetraethylammonium (TEA), N-methylphenylpyridinium (MPP+), paraquat, agmatine, cimetidine, procainamide, pramipexole, atenolol, serotonin, quinidine, verapamil, cisplatin, oxaliplatin, and pyrimethamine.
9. The method of embodiment 8, wherein the substrate is metformin.
10. A method for treating breast cancer in a subject comprising administering a therapeutically effective amount of tucatinib, or salt or solvate thereof, to the subject, wherein the subject is not concurrently receiving treatment with a therapeutically effective amount of a substrate of an organic cation transporter (OCT).
11. The method of embodiment 10, wherein the subject has not received treatment with the substrate of the OCT within the past 7 days.
12. The method of embodiment 10, wherein the subject has not received treatment with the substrate of the OCT within the past 3 months.
13. The method of embodiment 10, wherein the subject has not received treatment with the substrate of the OCT protein within the past 12 months.
14. The method of embodiment 10, wherein the subject has not previously received treatment with the substrate of the OCT.
15. The method of any one of embodiments 10-14, wherein the OCT is OCT1.
16. The method of any one of embodiments 10-14, wherein the OCT is OCT2.
17. The method of anyone of embodiments 10-16, wherein the substrate of the OCT is selected from the group consisting of metformin, oxazolidinone, fexofenadine, tetraethylammonium (TEA), N-methylphenylpyridinium (MPP+), paraquat, agmatine, cimetidine, procainamide, pramipexole, atenolol, serotonin, quinidine, verapamil, cisplatin, oxaliplatin, and pyrimethamine.
18. The method of embodiment 17, wherein the substrate is metformin.
19. The method of any one of embodiments 10-18, wherein the subject is not concurrently receiving treatment with a therapeutically effective amount of a substrate of a MATE protein.
20. The method of embodiment 19, wherein the subject has not received treatment with the substrate of the MATE protein within the past 7 days.
21. The method of embodiment 19, wherein the subject has not received treatment with the substrate of the MATE protein within the past 3 months.
22. The method of embodiment 19, wherein the subject has not received treatment with the substrate of the MATE protein within the past 12 months.
23. The method of embodiment 19, wherein the subject has not previously received treatment with the substrate of the MATE protein.
24. The method of any one of embodiments 19-23, wherein the MATE protein is MATE1.
25. The method of any one of embodiments 19-23, wherein the MATE protein is MATE2K.
26. A method for treating breast cancer in a subject comprising administering a therapeutically effective amount of tucatinib, or salt or solvate thereof, to the subject, wherein the subject does not have impaired renal function.
27. The method of embodiment 26, wherein the subject has not had impaired renal function within the past 12 months.
28. The method of any one of embodiments 1-25, wherein the subject does not have impaired renal function.
29. The method of embodiment 28, wherein the subject has not had impaired renal function within the past 12 months.
30. The method of any one of embodiments 26-29, wherein impaired renal function is determined based on the serum creatinine level in the subject.
31. The method of embodiment 30, wherein a) the subject is male and the subject has a serum creatinine level of less than 1.5 mg/dL or b) the subject is female and has a serum creatinine level of less than to 1.4 mg/dL.
32. The method of any one of embodiments 26-29, wherein impaired renal function is determined based on the subject having abnormal creatinine clearance.
33. The method of any one of embodiments 26-29, wherein impaired renal function is determined based on the glomerular filtration rate of the subject.
34. The method of any one of embodiments 1-33, wherein the subject is not concurrently receiving treatment with a therapeutically effective amount of a compound that modulates the activity of a cytochrome p450 protein.
35. The method of embodiment 34, wherein the subject has not received treatment with the compound that modulates the activity of the cytochrome p450 protein within the past 7 days.
36. The method of embodiment 34, wherein the subject has not received treatment with the compound that modulates the activity of the cytochrome p450 protein within the past 3 months.
37. The method of embodiment 34, wherein the subject has not received treatment with the compound that modulates the activity of the cytochrome p450 protein within the past 12 months.
38. The method of embodiment 34, wherein the subject has not previously received treatment with compound that modulates the activity of the cytochrome p450 protein.
39. The method of any one of embodiments 34-38, wherein the compound that modulates the activity of the cytochrome p450 protein is an inhibitor of the activity of the cytochrome p450 protein.
40. The method of embodiment 39, wherein the cytochrome p450 protein is CYP3A4.
41. The method of embodiment 40, wherein the compound that inhibits the activity of CYP3A4 is itraconazole.
42. The method of embodiment 39, wherein the cytochrome p450 protein is CYP2C8.
43. The method of embodiment 42, wherein the compound that inhibits the activity of CYP2C8 is gemfibrozil.
44. The method of any one of embodiments 34-38, wherein the compound that modulates the activity of the cytochrome p450 protein is an inducer of the activity of the cytochrome p450 protein.
45. The method of embodiment 44, wherein the cytochrome p450 protein is CYP3A4.
46. The method of embodiment 44, wherein the cytochrome p450 protein is CYP2C8.
47. The method of any one of embodiments 44-46, wherein the compound that induces the activity of the cytochrome p450 protein is rifampin.
48. A method for treating breast cancer in a subject comprising administering a therapeutically effective amount of tucatinib, or salt or solvate thereof, to the subject, wherein the subject is not concurrently receiving treatment with a therapeutically effective amount of a compound that modulates the activity of a cytochrome p450 protein.
49. The method of embodiment 48, wherein the subject has not received treatment with the compound that modulates the activity of the cytochrome p450 protein within the past 7 days.
50. The method of embodiment 48, wherein the subject has not received treatment with the compound that modulates the activity of the cytochrome p450 protein within the past 3 months.
51. The method of embodiment 48, wherein the subject has not received treatment with the compound that modulates the activity of the cytochrome p450 protein within the past 12 months.
52. The method of embodiment 48, wherein the subject has not previously received treatment with compound that modulates the activity of the cytochrome p450 protein.
53. The method of any one of embodiments 48-52, wherein the compound that modulates the activity of the cytochrome p450 protein is an inhibitor of the activity of the cytochrome p450 protein.
54. The method of embodiment 53, wherein the cytochrome p450 protein is CYP3A4.
55. The method of embodiment 54, wherein the compound that inhibits the activity of CYP3A4 is itraconazole.
56. The method of embodiment 53, wherein the cytochrome p450 protein is CYP2C8.
57. The method of embodiment 56, wherein the compound that inhibits the activity of CYP2C8 is gemfibrozil.
58. The method of any one of embodiments 48-52, wherein the compound that modulates the activity of the cytochrome p450 protein is an inducer of the activity of the cytochrome p450 protein.
59. The method of embodiment 58, wherein the cytochrome p450 protein is CYP3A4.
60. The method of embodiment 58, wherein the cytochrome p450 protein is CYP2C8.
61. The method of any one of embodiments 58-60, wherein the compound that induces the activity of the cytochrome p450 protein is rifampin.
62. The method of any one of embodiments 1-61, wherein the tucatinib is administered to the subject at a dose of about 150 mg to about 650 mg.
63. The method of embodiment 62, wherein the tucatinib is administered to the subject at a dose of about 300 mg.
64. The method of embodiment 62 or embodiment 63, wherein the tucatinib is administered once or twice per day.
65. The method of embodiment 64, wherein the tucatinib is administered to the subject at a dose of about 300 mg twice per day.
66. The method of any one of embodiments 1-65, wherein the tucatinib is administered to the subject orally.
67. The method of any one of embodiments 1-66, wherein the breast cancer is a HER2 positive breast cancer.
68. The method of embodiment 67, wherein the cancer is determined to be HER2 positive using in situ hybridization, fluorescence in situ hybridization, or immunohistochemistry.
69. The method of any one of embodiments 1-68, wherein the breast cancer is metastatic.
70. The method of embodiment 69, wherein the breast cancer has metastasized to the brain.
71. The method of any one of embodiments 1-70, wherein the breast cancer is locally advanced.
72. The method of any one of embodiments 1-71, wherein the breast cancer is unresectable.
73. The method of any one of embodiments 1-72, further comprising administering one or more additional therapeutic agents to the subject to treat the breast cancer.
74. The method of embodiment 73, wherein the one or more additional therapeutic agents is selected from the group consisting of capecitabine and an anti-HER2 antibody.
75. The method of embodiment 73, wherein the one or more additional therapeutic agents is capecitabine.
76. The method of embodiment 73, wherein the one or more additional therapeutic agents is trastuzumab.
77. The method of embodiment 73, wherein the one or more additional therapeutic agents are capecitabine and trastuzumab.
78. The method of embodiment 75 or embodiment 77, wherein the capecitabine is administered to the subject at a dose of about 500 mg/m² to about 1500 mg/m².
79. The method of embodiment 78, wherein the capecitabine is administered to the subject at a dose of about 1000 mg/m².
80. The method of embodiment 78 or embodiment 79, wherein the capecitabine is administered to the subject orally.
81. The method of any one of embodiments 77-80, wherein the capecitabine is administered to the subject twice per day.
82. The method of embodiment 76 or embodiment 77, wherein the trastuzumab is administered to the subject at a dose of about 400 mg to about 800 mg.
83. The method of embodiment 82, wherein the trastuzumab is administered to the subject at a dose of about 600 mg
84. The method of embodiment 82 or embodiment 83, wherein the trastuzumab is administered to the subject subcutaneously.
85. The method of embodiment 76 or embodiment 77, wherein the trastuzumab is administered to the subject at a dose of about 4 mg/kg to about 10 mg/kg.
86. The method of embodiment 85, wherein the trastuzumab is administered to the subject at a dose of about 6 mg/kg.
87. The method of embodiment 85, wherein the trastuzumab is administered to the subject at a dose of about 8 mg/kg.
88. The method of embodiment 85, wherein the trastuzumab is administered to the subject at an initial dose of about 8 mg/kg followed by subsequent doses of about 6 mg/kg.
89. The method of any one of embodiments 85-88, wherein the trastuzumab is administered intravenously.
90. The method of any one of embodiments 82-89, wherein the trastuzumab is administered once about every 1 week, once about every 2 weeks, once about every 3 weeks, or once about every 4 weeks.
91. The method of embodiment 90, wherein the trastuzumab is administered once about every 3 weeks.
92. The method of embodiment 77, wherein the tucatinib, capecitabine and trastuzumab are administered to the subject on a 21 day treatment cycle.
93. The method of embodiment 92, wherein the tucatinib is administered to the subject twice per day on each day of the 21 day treatment cycle.
94. The method of embodiment 92 or 93, wherein the capecitabine is administered to the subject twice per day on each of days 1-14 of the 21 day treatment cycle.
95. The method of any one of embodiments 92-94, wherein the trastuzumab is administered to the subject once per 21 day treatment cycle.
96. The method of embodiment 95, wherein the dose of trastuzumab during the first 21 day treatment cycle is 8 mg/kg and the dose of trastuzumab during the subsequent 21 day treatment cycles is 6 mg/kg.
97. The method of any one of embodiments 1-96, wherein the subject has been previously treated with one or more additional therapeutic agents for the breast cancer.
98. The method of embodiment 97, wherein the one or more additional therapeutic agents is an anti-HER2 antibody or anti-HER2 antibody-drug conjugate.
99. The method of embodiment 98, wherein the one or more additional therapeutic agents is trastuzumab, pertuzumab and/or T-DM1.
100. The method of any one of embodiments 1-99, wherein the subject has not been treated with another therapeutic agent for the breast cancer within the past 12 months.
101. The method of any one of embodiments 1-96, wherein the subject has not previously been treated with another therapeutic agent for the breast cancer.
102. The method of any one of embodiments 1-101, wherein the subject has not previously been treated with lapatinib, neratinib, afatinib, or capecitabine.
103. The method of any one of embodiments 1-102, wherein treating the subject results in a tumor growth inhibition (TGI) index of at least about 85%.
104. The method of any one of embodiments 1-102, wherein treating the subject results in a TGI index of about 100%.
105. The method of any one of embodiments 1-104, wherein one or more therapeutic effects in the subject is improved after administration of tucatinib to the subject relative to a baseline.
106. The method of embodiment 105, wherein the one or more therapeutic effects is selected from the group consisting of: size of a tumor derived from the breast cancer, objective response rate, duration of response, time to response, progression free survival and overall survival.
107. The method of any one of embodiments 1-106, wherein the size of a tumor derived from the breast cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the breast cancer before administration of tucatinib to the subject.
108. The method of any one of embodiments 1-107, wherein the objective response rate is at least about 20%, at least about 25%, at least about 30%, at least about 35%, at least about 40%, at least about 45%, at least about 50%, at least about 60%, at least about 70%, or at least about 80%.
109. The method of any one of embodiments 1-108, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of tucatinib to the subject.
110. The method of any one of embodiments 1-109, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of tucatinib to the subject.
111. The method of any one of embodiments 1-110, wherein the duration of response to tucatinib is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of tucatinib to the subject.
112. The method of any one of embodiments 1-111, wherein the subject is a human.
113. A therapeutically effective amount of tucatinib, or salt or solvate thereof, for use in the treatment of breast cancer in a subject, wherein the subject is not concurrently receiving treatment with a therapeutically effective amount of a substrate of a multidrug and toxin extrusion (MATE) protein.
114. The therapeutically effective amount of tucatinib, or salt or solvate thereof, for use of embodiment 113, wherein the subject has not received treatment with the substrate of the MATE protein within the past 7 days.
115. The therapeutically effective amount of tucatinib, or salt or solvate thereof, for use of embodiment 113, wherein the subject has not received treatment with the substrate of the MATE protein within the past 3 months.
116. The therapeutically effective amount of tucatinib, or salt or solvate thereof, for use of embodiment 113, wherein the subject has not received treatment with the substrate of the MATE protein within the past 12 months.
117. The therapeutically effective amount of tucatinib, or salt or solvate thereof, for use embodiment 113, wherein the subject has not previously received treatment with the substrate of the MATE protein.
118. The therapeutically effective amount of tucatinib, or salt or solvate thereof, for use of any one of embodiments 113-117, wherein the MATE protein is MATE1.
119. The therapeutically effective amount of tucatinib, or salt or solvate thereof, for use of any one of embodiments 113-117, wherein the MATE protein is MATE2K.
120. The therapeutically effective amount of tucatinib, or salt or solvate thereof, for use of anyone of embodiments 113-119, wherein the substrate of the MATE protein is selected from the group consisting of metformin, oxazolidinone, fexofenadine, tetraethylammonium (TEA), N-methylphenylpyridinium (MPP+), paraquat, agmatine, cimetidine, procainamide, pramipexole, atenolol, serotonin, quinidine, verapamil, cisplatin, oxaliplatin, and pyrimethamine.
121. The therapeutically effective amount of tucatinib, or salt or solvate thereof, for use of embodiment 120, wherein the substrate is metformin.
122. A therapeutically effective amount of tucatinib, or salt or solvate thereof, for use in the treatment of breast cancer in a subject, wherein the subject is not concurrently receiving treatment with a therapeutically effective amount of a substrate of an organic cation transporter (OCT).
123. The therapeutically effective amount of tucatinib, or salt or solvate thereof, for use of embodiment 122, wherein the subject has not received treatment with the substrate of the OCT within the past 7 days.
124. The therapeutically effective amount of tucatinib, or salt or solvate thereof, for use of embodiment 122, wherein the subject has not received treatment with the substrate of the OCT within the past 3 months.
125. The therapeutically effective amount of tucatinib, or salt or solvate thereof, for use of embodiment 122, wherein the subject has not received treatment with the substrate of the OCT protein within the past 12 months.
126. The therapeutically effective amount of tucatinib, or salt or solvate thereof, for use of embodiment 122, wherein the subject has not previously received treatment with the substrate of the OCT.
127. The therapeutically effective amount of tucatinib, or salt or solvate thereof, for use of any one of embodiments 122-126, wherein the OCT is OCT1.
128. The therapeutically effective amount of tucatinib, or salt or solvate thereof, for use of any one of embodiments 122-126, wherein the OCT is OCT2.
129. The therapeutically effective amount of tucatinib, or salt or solvate thereof, for use of anyone of embodiments 122-128, wherein the substrate of the OCT is selected from the group consisting of metformin, oxazolidinone, fexofenadine, tetraethylammonium (TEA), N-methylphenylpyridinium (MPP+), paraquat, agmatine, cimetidine, procainamide, pramipexole, atenolol, serotonin, quinidine, verapamil, cisplatin, oxaliplatin, and pyrimethamine.
130. The therapeutically effective amount of tucatinib, or salt or solvate thereof, for use of embodiment 129, wherein the substrate is metformin.
131. The therapeutically effective amount of tucatinib, or salt or solvate thereof, for use of any one of embodiments 122-130, wherein the subject is not concurrently receiving treatment with a therapeutically effective amount of a substrate of a MATE protein.
132. The therapeutically effective amount of tucatinib, or salt or solvate thereof, for use of embodiment 131, wherein the subject has not received treatment with the substrate of the MATE protein within the past 7 days.
133. The therapeutically effective amount of tucatinib, or salt or solvate thereof, for use of embodiment 131, wherein the subject has not received treatment with the substrate of the MATE protein within the past 3 months.
134. The therapeutically effective amount of tucatinib, or salt or solvate thereof, for use of embodiment 131, wherein the subject has not received treatment with the substrate of the MATE protein within the past 12 months.
135. The therapeutically effective amount of tucatinib, or salt or solvate thereof, for use of embodiment 131, wherein the subject has not previously received treatment with the substrate of the MATE protein.
136. The therapeutically effective amount of tucatinib, or salt or solvate thereof, for use of any one of embodiments 131-135, wherein the MATE protein is MATE1.
137. The therapeutically effective amount of tucatinib, or salt or solvate thereof, for use of any one of embodiments 131-135, wherein the MATE protein is MATE2K.
138. A therapeutically effective amount of tucatinib, or salt or solvate thereof, for use in the treatment of breast cancer in a subject, wherein the subject does not have impaired renal function.
139. The therapeutically effective amount of tucatinib, or salt or solvate thereof, for use of embodiment 138, wherein the subject has not had impaired renal function within the past 12 months.
140. The therapeutically effective amount of tucatinib, or salt or solvate thereof, for use of any one of embodiments 113-137, wherein the subject does not have impaired renal function.
141. The therapeutically effective amount of tucatinib, or salt or solvate thereof, for use of embodiment 140, wherein the subject has not had impaired renal function within the past 12 months.
142. The therapeutically effective amount of tucatinib, or salt or solvate thereof, for use of any one of embodiments 138-141, wherein impaired renal function is determined based on the serum creatinine level in the subject.
143. The therapeutically effective amount of tucatinib, or salt or solvate thereof, for use of embodiment 142, wherein a) the subject is male and the subject has a serum creatinine level of less than 1.5 mg/dL or b) the subject is female and has a serum creatinine level of less than to 1.4 mg/dL.
144. The therapeutically effective amount of tucatinib, or salt or solvate thereof, for use of any one of embodiments 138-141, wherein impaired renal function is determined based on the subject having abnormal creatinine clearance.
145. The therapeutically effective amount of tucatinib, or salt or solvate thereof, for use of any one of embodiments 138-141, wherein impaired renal function is determined based on the glomerular filtration rate of the subject.
146. The therapeutically effective amount of tucatinib, or salt or solvate thereof, for use of any one of embodiments 113-145, wherein the subject is not concurrently receiving treatment with a therapeutically effective amount of a compound that modulates the activity of a cytochrome p450 protein.
147. The therapeutically effective amount of tucatinib, or salt or solvate thereof, for use of embodiment 146, wherein the subject has not received treatment with the compound that modulates the activity of the cytochrome p450 protein within the past 7 days.
148. The therapeutically effective amount of tucatinib, or salt or solvate thereof, for use of embodiment 146, wherein the subject has not received treatment with the compound that modulates the activity of the cytochrome p450 protein within the past 3 months.
149. The therapeutically effective amount of tucatinib, or salt or solvate thereof, for use of embodiment 146, wherein the subject has not received treatment with the compound that modulates the activity of the cytochrome p450 protein within the past 12 months.
150. The therapeutically effective amount of tucatinib, or salt or solvate thereof, for use of embodiment 146, wherein the subject has not previously received treatment with compound that modulates the activity of the cytochrome p450 protein.
151. The therapeutically effective amount of tucatinib, or salt or solvate thereof, for use of any one of embodiments 145-150, wherein the compound that modulates the activity of the cytochrome p450 protein is an inhibitor of the activity of the cytochrome p450 protein.
152. The therapeutically effective amount of tucatinib, or salt or solvate thereof, for use of embodiment 151, wherein the cytochrome p450 protein is CYP3A4.
153. The therapeutically effective amount of tucatinib, or salt or solvate thereof, for use of embodiment 152, wherein the compound that inhibits the activity of CYP3A4 is itraconazole.
154. The therapeutically effective amount of tucatinib, or salt or solvate thereof, for use of embodiment 151, wherein the cytochrome p450 protein is CYP2C8.
155. The therapeutically effective amount of tucatinib, or salt or solvate thereof, for use of embodiment 154, wherein the compound that inhibits the activity of CYP2C8 is gemfibrozil.
156. The therapeutically effective amount of tucatinib, or salt or solvate thereof, for use of any one of embodiments 145-150, wherein the compound that modulates the activity of the cytochrome p450 protein is an inducer of the activity of the cytochrome p450 protein.
157. The therapeutically effective amount of tucatinib, or salt or solvate thereof, for use of embodiment 156, wherein the cytochrome p450 protein is CYP3A4.
158. The therapeutically effective amount of tucatinib, or salt or solvate thereof, for use of embodiment 156, wherein the cytochrome p450 protein is CYP2C8.
159. The therapeutically effective amount of tucatinib, or salt or solvate thereof, for use of any one of embodiments 156-158, wherein the compound that induces the activity of the cytochrome p450 protein is rifampin.
160. A therapeutically effective amount of tucatinib, or salt or solvate thereof, for use in the treatment of breast cancer in a subject, wherein the subject is not concurrently receiving treatment with a therapeutically effective amount of a compound that modulates the activity of a cytochrome p450 protein.
161. The therapeutically effective amount of tucatinib, or salt or solvate thereof, for use of embodiment 160, wherein the subject has not received treatment with the compound that modulates the activity of the cytochrome p450 protein within the past 7 days.
162. The therapeutically effective amount of tucatinib, or salt or solvate thereof, for use of embodiment 160, wherein the subject has not received treatment with the compound that modulates the activity of the cytochrome p450 protein within the past 3 months.
163. The therapeutically effective amount of tucatinib, or salt or solvate thereof, for use of embodiment 160, wherein the subject has not received treatment with the compound that modulates the activity of the cytochrome p450 protein within the past 12 months.
164. The therapeutically effective amount of tucatinib, or salt or solvate thereof, for use of embodiment 160, wherein the subject has not previously received treatment with compound that modulates the activity of the cytochrome p450 protein.
165. The therapeutically effective amount of tucatinib, or salt or solvate thereof, for use of any one of embodiments 160-164, wherein the compound that modulates the activity of the cytochrome p450 protein is an inhibitor of the activity of the cytochrome p450 protein.
166. The therapeutically effective amount of tucatinib, or salt or solvate thereof, for use of embodiment 165, wherein the cytochrome p450 protein is CYP3A4.
167. The therapeutically effective amount of tucatinib, or salt or solvate thereof, for use of embodiment 166, wherein the compound that inhibits the activity of CYP3A4 is itraconazole.
168. The therapeutically effective amount of tucatinib, or salt or solvate thereof, for use of embodiment 165, wherein the cytochrome p450 protein is CYP2C8.
169. The therapeutically effective amount of tucatinib, or salt or solvate thereof, for use of embodiment 168, wherein the compound that inhibits the activity of CYP2C8 is gemfibrozil.
170. The therapeutically effective amount of tucatinib, or salt or solvate thereof, for use of any one of embodiments 160-164, wherein the compound that modulates the activity of the cytochrome p450 protein is an inducer of the activity of the cytochrome p450 protein.
171. The therapeutically effective amount of tucatinib, or salt or solvate thereof, for use of embodiment 170, wherein the cytochrome p450 protein is CYP3A4.
172. The therapeutically effective amount of tucatinib, or salt or solvate thereof, for use of embodiment 170, wherein the cytochrome p450 protein is CYP2C8.
173. The therapeutically effective amount of tucatinib, or salt or solvate thereof, for use of any one of embodiments 170-172, wherein the compound that induces the activity of the cytochrome p450 protein is rifampin.
174. The therapeutically effective amount of tucatinib, or salt or solvate thereof, for use of any one of embodiments 113-173, wherein the tucatinib is administered to the subject at a dose of about 150 mg to about 650 mg.
175. The therapeutically effective amount of tucatinib, or salt or solvate thereof, for use of embodiment 174, wherein the tucatinib is administered to the subject at a dose of about 300 mg.
176. The therapeutically effective amount of tucatinib, or salt or solvate thereof, for use of embodiment 174 or embodiment 63, wherein the tucatinib is administered once or twice per day.
177. The therapeutically effective amount of tucatinib, or salt or solvate thereof, for use of embodiment 176, wherein the tucatinib is administered to the subject at a dose of about 300 mg twice per day.
178. The therapeutically effective amount of tucatinib, or salt or solvate thereof, for use of any one of embodiments 113-177, wherein the tucatinib is administered to the subject orally.
179. The therapeutically effective amount of tucatinib, or salt or solvate thereof, for use of any one of embodiments 113-178, wherein the breast cancer is a HER2 positive breast cancer.
180. The therapeutically effective amount of tucatinib, or salt or solvate thereof, for use of embodiment 179, wherein the cancer is determined to be HER2 positive using in situ hybridization, fluorescence in situ hybridization, or immunohistochemistry.
181. The therapeutically effective amount of tucatinib, or salt or solvate thereof, for use of any one of embodiments 113-180, wherein the breast cancer is metastatic.
182. The therapeutically effective amount of tucatinib, or salt or solvate thereof, for use of embodiment 181, wherein the breast cancer has metastasized to the brain.
183. The therapeutically effective amount of tucatinib, or salt or solvate thereof, for use of any one of embodiments 113-182, wherein the breast cancer is locally advanced.
184. The therapeutically effective amount of tucatinib, or salt or solvate thereof, for use of any one of embodiments 113-183, wherein the breast cancer is unresectable.
185. The therapeutically effective amount of tucatinib, or salt or solvate thereof, for use of any one of embodiments 113-184, wherein the tucatinib is for administration, or to be administered in combination with one or more additional therapeutic agents to treat the breast cancer.
186. The therapeutically effective amount of tucatinib, or salt or solvate thereof, for use of embodiment 185, wherein the one or more additional therapeutic agents is selected from the group consisting of capecitabine and an anti-HER2 antibody.
187. The therapeutically effective amount of tucatinib, or salt or solvate thereof, for use of embodiment 185, wherein the one or more additional therapeutic agents is capecitabine.
188. The therapeutically effective amount of tucatinib, or salt or solvate thereof, for use of embodiment 185, wherein the one or more additional therapeutic agents is trastuzumab.
189. The therapeutically effective amount of tucatinib, or salt or solvate thereof, for use of embodiment 185, wherein the one or more additional therapeutic agents are capecitabine and trastuzumab.
190. The therapeutically effective amount of tucatinib, or salt or solvate thereof, for use of embodiment 187 or embodiment 189, wherein the capecitabine is administered to the subject at a dose of about 500 mg/m² to about 1500 mg/m².
191. The therapeutically effective amount of tucatinib, or salt or solvate thereof, for use of embodiment 190, wherein the capecitabine is administered to the subject at a dose of about 1000 mg/m².
192. The therapeutically effective amount of tucatinib, or salt or solvate thereof, for use of embodiment 190 or embodiment 191, wherein the capecitabine is administered to the subject orally.
193. The therapeutically effective amount of tucatinib, or salt or solvate thereof, for use of any one of embodiments 189-192, wherein the capecitabine is administered to the subject twice per day.
194. The therapeutically effective amount of tucatinib, or salt or solvate thereof, for use of embodiment 188 or embodiment 189, wherein the trastuzumab is administered to the subject at a dose of about 400 mg to about 800 mg.
195. The therapeutically effective amount of tucatinib, or salt or solvate thereof, for use of embodiment 194, wherein the trastuzumab is administered to the subject at a dose of about 600 mg
196. The therapeutically effective amount of tucatinib, or salt or solvate thereof, for use of embodiment 194 or embodiment 195, wherein the trastuzumab is administered to the subject subcutaneously.
197. The therapeutically effective amount of tucatinib, or salt or solvate thereof, for use of embodiment 188 or embodiment 189, wherein the trastuzumab is administered to the subject at a dose of about 4 mg/kg to about 10 mg/kg.
198. The therapeutically effective amount of tucatinib, or salt or solvate thereof, for use of embodiment 197, wherein the trastuzumab is administered to the subject at a dose of about 6 mg/kg.
199. The therapeutically effective amount of tucatinib, or salt or solvate thereof, for use of embodiment 197, wherein the trastuzumab is administered to the subject at a dose of about 8 mg/kg.
200. The therapeutically effective amount of tucatinib, or salt or solvate thereof, for use of embodiment 197, wherein the trastuzumab is administered to the subject at an initial dose of about 8 mg/kg followed by subsequent doses of about 6 mg/kg.
201. The therapeutically effective amount of tucatinib, or salt or solvate thereof, for use of any one of embodiments 197-200, wherein the trastuzumab is administered intravenously.
202. The therapeutically effective amount of tucatinib, or salt or solvate thereof, for use of any one of embodiments 194-201, wherein the trastuzumab is administered once about every 1 week, once about every 2 weeks, once about every 3 weeks, or once about every 4 weeks.
203. The therapeutically effective amount of tucatinib, or salt or solvate thereof, for use of embodiment 202, wherein the trastuzumab is administered once about every 3 weeks.
204. The therapeutically effective amount of tucatinib, or salt or solvate thereof, for use of embodiment 189, wherein the tucatinib, capecitabine and trastuzumab are administered to the subject on a 21 day treatment cycle.
205. The therapeutically effective amount of tucatinib, or salt or solvate thereof, for use of embodiment 204, wherein the tucatinib is administered to the subject twice per day on each day of the 21 day treatment cycle.
206. The therapeutically effective amount of tucatinib, or salt or solvate thereof, for use of embodiment 204 or 205, wherein the capecitabine is administered to the subject twice per day on each of days 1-14 of the 21 day treatment cycle.
207. The therapeutically effective amount of tucatinib, or salt or solvate thereof, for use of any one of embodiments 204-206, wherein the trastuzumab is administered to the subject once per 21 day treatment cycle.
208. The therapeutically effective amount of tucatinib, or salt or solvate thereof, for use of embodiment 207, wherein the dose of trastuzumab during the first 21 day treatment cycle is 8 mg/kg and the dose of trastuzumab during the subsequent 21 day treatment cycles is 6 mg/kg.
209. The therapeutically effective amount of tucatinib, or salt or solvate thereof, for use of any one of embodiments 113-208, wherein the subject has been previously treated with one or more additional therapeutic agents for the breast cancer.
210. The therapeutically effective amount of tucatinib, or salt or solvate thereof, for use of embodiment 209, wherein the one or more additional therapeutic agents is an anti-HER2 antibody or anti-HER2 antibody-drug conjugate.
211. The therapeutically effective amount of tucatinib, or salt or solvate thereof, for use of embodiment 210, wherein the one or more additional therapeutic agents is trastuzumab, pertuzumab and/or T-DM1.
212. The therapeutically effective amount of tucatinib, or salt or solvate thereof, for use of any one of embodiments 113-211, wherein the subject has not been treated with another therapeutic agent for the breast cancer within the past 12 months.
213. The therapeutically effective amount of tucatinib, or salt or solvate thereof, for use of any one of embodiments 113-208, wherein the subject has not previously been treated with another therapeutic agent for the breast cancer.
214. The therapeutically effective amount of tucatinib, or salt or solvate thereof, for use of any one of embodiments 113-213, wherein the subject has not previously been treated with lapatinib, neratinib, afatinib, or capecitabine.
215. The therapeutically effective amount of tucatinib, or salt or solvate thereof, for use of any one of embodiments 113-214, wherein treating the subject results in a tumor growth inhibition (TGI) index of at least about 85%.
216. The therapeutically effective amount of tucatinib, or salt or solvate thereof, for use of any one of embodiments 113-214, wherein treating the subject results in a TGI index of about 100%.
217. The therapeutically effective amount of tucatinib, or salt or solvate thereof, for use of any one of embodiments 113-216, wherein one or more therapeutic effects in the subject is improved after administration of tucatinib to the subject relative to a baseline.
218. The therapeutically effective amount of tucatinib, or salt or solvate thereof, for use of embodiment 217, wherein the one or more therapeutic effects is selected from the group consisting of: size of a tumor derived from the breast cancer, objective response rate, duration of response, time to response, progression free survival and overall survival.
219. The therapeutically effective amount of tucatinib, or salt or solvate thereof, for use of any one of embodiments 113-218, wherein the size of a tumor derived from the breast cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the breast cancer before administration of tucatinib to the subject.
220. The therapeutically effective amount of tucatinib, or salt or solvate thereof, for use of any one of embodiments 113-219, wherein the objective response rate is at least about 20%, at least about 25%, at least about 30%, at least about 35%, at least about 40%, at least about 45%, at least about 50%, at least about 60%, at least about 70%, or at least about 80%.
221. The therapeutically effective amount of tucatinib, or salt or solvate thereof, for use of any one of embodiments 113-220, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of tucatinib to the subject.
222. The therapeutically effective amount of tucatinib, or salt or solvate thereof, for use of any one of embodiments 113-221, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of tucatinib to the subject.
223. The therapeutically effective amount of tucatinib, or salt or solvate thereof, for use of any one of embodiments 113-222, wherein the duration of response to tucatinib is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of tucatinib to the subject.
224. The therapeutically effective amount of tucatinib, or salt or solvate thereof, for use of any one of embodiments 113-223, wherein the subject is a human.
225. Use of a therapeutically effective amount of tucatinib, or salt or solvate thereof, for the manufacture of a medicament for treating breast cancer in a subject, wherein the subject is not concurrently receiving treatment with a therapeutically effective amount of a substrate of a multidrug and toxin extrusion (MATE) protein.
226. The use of embodiment 225, wherein the subject has not received treatment with the substrate of the MATE protein within the past 7 days.
227. The use of embodiment 225, wherein the subject has not received treatment with the substrate of the MATE protein within the past 3 months.
228. The use of embodiment 225, wherein the subject has not received treatment with the substrate of the MATE protein within the past 12 months.
229. The use of embodiment 225, wherein the subject has not previously received treatment with the substrate of the MATE protein.
230. The use of any one of embodiments 225-229, wherein the MATE protein is MATE1.
231. The use of any one of embodiments 225-229, wherein the MATE protein is MATE2K.
232. The use of anyone of embodiments 225-231, wherein the substrate of the MATE protein is selected from the group consisting of metformin, oxazolidinone, fexofenadine, tetraethylammonium (TEA), N-methylphenylpyridinium (MPP+), paraquat, agmatine, cimetidine, procainamide, pramipexole, atenolol, serotonin, quinidine, verapamil, cisplatin, oxaliplatin, and pyrimethamine.
233. The use of embodiment 232, wherein the substrate is metformin.
234. Use of a therapeutically effective amount of tucatinib, or salt or solvate thereof, for the manufacture of a medicament for treating breast cancer in a subject, wherein the subject is not concurrently receiving treatment with a therapeutically effective amount of a substrate of an organic cation transporter (OCT).
235. The use of embodiment 234, wherein the subject has not received treatment with the substrate of the OCT within the past 7 days.
236. The use of embodiment 234, wherein the subject has not received treatment with the substrate of the OCT within the past 3 months.
237. The use of embodiment 234, wherein the subject has not received treatment with the substrate of the OCT protein within the past 12 months.
238. The use of embodiment 234, wherein the subject has not previously received treatment with the substrate of the OCT.
239. The use of any one of embodiments 234-238, wherein the OCT is OCT1.
240. The use of any one of embodiments 234-238, wherein the OCT is OCT2.
241. The use of anyone of embodiments 234-240, wherein the substrate of the OCT is selected from the group consisting of metformin, oxazolidinone, fexofenadine, tetraethylammonium (TEA), N-methylphenylpyridinium (MPP+), paraquat, agmatine, cimetidine, procainamide, pramipexole, atenolol, serotonin, quinidine, verapamil, cisplatin, oxaliplatin, and pyrimethamine.
242. The use of embodiment 241, wherein the substrate is metformin.
243. The use of any one of embodiments 234-242, wherein the subject is not concurrently receiving treatment with a therapeutically effective amount of a substrate of a MATE protein.
244. The use of embodiment 243, wherein the subject has not received treatment with the substrate of the MATE protein within the past 7 days.
245. The use of embodiment 243, wherein the subject has not received treatment with the substrate of the MATE protein within the past 3 months.
246. The use of embodiment 243, wherein the subject has not received treatment with the substrate of the MATE protein within the past 12 months.
247. The use of embodiment 243, wherein the subject has not previously received treatment with the substrate of the MATE protein.
248. The use of any one of embodiments 243-247, wherein the MATE protein is MATE1.
249. The use of any one of embodiments 243-247, wherein the MATE protein is MATE2K.
250. Use of a therapeutically effective amount of tucatinib, or salt or solvate thereof, for the manufacture of a medicament for treating breast cancer in a subject, wherein the subject does not have impaired renal function.
251. The use of embodiment 250, wherein the subject has not had impaired renal function within the past 12 months.
252. The use of any one of embodiments 225-249, wherein the subject does not have impaired renal function.
253. The use of embodiment 252, wherein the subject has not had impaired renal function within the past 12 months.
254. The use of any one of embodiments 250-253, wherein impaired renal function is determined based on the serum creatinine level in the subject.
255. The use of embodiment 254, wherein a) the subject is male and the subject has a serum creatinine level of less than 1.5 mg/dL or b) the subject is female and has a serum creatinine level of less than to 1.4 mg/dL.
256. The use of any one of embodiments 250-253, wherein impaired renal function is determined based on the subject having abnormal creatinine clearance.
257. The use of any one of embodiments 250-253, wherein impaired renal function is determined based on the glomerular filtration rate of the subject.
258. The use of any one of embodiments 225-257, wherein the subject is not concurrently receiving treatment with a therapeutically effective amount of a compound that modulates the activity of a cytochrome p450 protein.
259. The use of embodiment 258, wherein the subject has not received treatment with the compound that modulates the activity of the cytochrome p450 protein within the past 7 days.
260. The use of embodiment 258, wherein the subject has not received treatment with the compound that modulates the activity of the cytochrome p450 protein within the past 3 months.
261. The use of embodiment 258, wherein the subject has not received treatment with the compound that modulates the activity of the cytochrome p450 protein within the past 12 months.
262. The use of embodiment 258, wherein the subject has not previously received treatment with compound that modulates the activity of the cytochrome p450 protein.
263. The use of any one of embodiments 258-262, wherein the compound that modulates the activity of the cytochrome p450 protein is an inhibitor of the activity of the cytochrome p450 protein.
264. The use of embodiment 263, wherein the cytochrome p450 protein is CYP3A4.
265. The use of embodiment 264, wherein the compound that inhibits the activity of CYP3A4 is itraconazole.
266. The use of embodiment 263, wherein the cytochrome p450 protein is CYP2C8.
267. The use of embodiment 266, wherein the compound that inhibits the activity of CYP2C8 is gemfibrozil.
268. The use of any one of embodiments 258-262, wherein the compound that modulates the activity of the cytochrome p450 protein is an inducer of the activity of the cytochrome p450 protein.
269. The use of embodiment 268, wherein the cytochrome p450 protein is CYP3A4.
270. The use of embodiment 268, wherein the cytochrome p450 protein is CYP2C8.
271. The use of any one of embodiments 268-270, wherein the compound that induces the activity of the cytochrome p450 protein is rifampin.
272. Use of a therapeutically effective amount of tucatinib, or salt or solvate thereof, for the manufacture of a medicament for treating breast cancer in a subject, wherein the subject is not concurrently receiving treatment with a therapeutically effective amount of a compound that modulates the activity of a cytochrome p450 protein.
273. The use of embodiment 4272, wherein the subject has not received treatment with the compound that modulates the activity of the cytochrome p450 protein within the past 7 days.
274. The use of embodiment 272, wherein the subject has not received treatment with the compound that modulates the activity of the cytochrome p450 protein within the past 3 months.
275. The use of embodiment 272, wherein the subject has not received treatment with the compound that modulates the activity of the cytochrome p450 protein within the past 12 months.
276. The use of embodiment 272, wherein the subject has not previously received treatment with compound that modulates the activity of the cytochrome p450 protein.
277. The use of any one of embodiments 272-276, wherein the compound that modulates the activity of the cytochrome p450 protein is an inhibitor of the activity of the cytochrome p450 protein.
278. The use of embodiment 277, wherein the cytochrome p450 protein is CYP3A4.
279. The use of embodiment 278, wherein the compound that inhibits the activity of CYP3A4 is itraconazole.
280. The use of embodiment 277, wherein the cytochrome p450 protein is CYP2C8.
281. The use of embodiment 280, wherein the compound that inhibits the activity of CYP2C8 is gemfibrozil.
282. The use of any one of embodiments 272-276, wherein the compound that modulates the activity of the cytochrome p450 protein is an inducer of the activity of the cytochrome p450 protein.
283. The use of embodiment 282, wherein the cytochrome p450 protein is CYP3A4.
284. The use of embodiment 282, wherein the cytochrome p450 protein is CYP2C8.
285. The use of any one of embodiments 282-284, wherein the compound that induces the activity of the cytochrome p450 protein is rifampin.
286. The use of any one of embodiments 225-285, wherein the tucatinib is administered to the subject at a dose of about 150 mg to about 650 mg.
287. The use of embodiment 286, wherein the tucatinib is administered to the subject at a dose of about 300 mg.
288. The use of embodiment 286 or embodiment 287, wherein the tucatinib is administered once or twice per day.
289. The use of embodiment 288, wherein the tucatinib is administered to the subject at a dose of about 300 mg twice per day.
290. The use of any one of embodiments 225-289, wherein the tucatinib is administered to the subject orally.
291. The use of any one of embodiments 225-290, wherein the breast cancer is a HER2 positive breast cancer.
292. The use of embodiment 291, wherein the cancer is determined to be HER2 positive using in situ hybridization, fluorescence in situ hybridization, or immunohistochemistry.
293. The use of any one of embodiments 225-292, wherein the breast cancer is metastatic.
294. The use of embodiment 293, wherein the breast cancer has metastasized to the brain.
295. The use of any one of embodiments 225-7294, wherein the breast cancer is locally advanced.
296. The use of any one of embodiments 225-295, wherein the breast cancer is unresectable.
297. The use of any one of embodiments 225-296, wherein the medicament is for use in combination with one or more additional therapeutic agents to treat the breast cancer.
298. The use of embodiment 297, wherein the one or more additional therapeutic agents is selected from the group consisting of capecitabine and an anti-HER2 antibody.
299. The use of embodiment 297, wherein the one or more additional therapeutic agents is capecitabine.
300. The use of embodiment 297, wherein the one or more additional therapeutic agents is trastuzumab.
301. The use of embodiment 297, wherein the one or more additional therapeutic agents are capecitabine and trastuzumab.
302. The use of embodiment 299 or embodiment 301, wherein the capecitabine is administered to the subject at a dose of about 500 mg/m² to about 1500 mg/m².
303. The use of embodiment 302, wherein the capecitabine is administered to the subject at a dose of about 1000 mg/m².
304. The use of embodiment 302 or embodiment 303, wherein the capecitabine is administered to the subject orally.
305. The use of any one of embodiments 301-304, wherein the capecitabine is administered to the subject twice per day.
306. The use of embodiment 300 or embodiment 7301, wherein the trastuzumab is administered to the subject at a dose of about 400 mg to about 800 mg.
307. The use of embodiment 306, wherein the trastuzumab is administered to the subject at a dose of about 600 mg
308. The use of embodiment 306 or embodiment 307, wherein the trastuzumab is administered to the subject subcutaneously.
309. The use of embodiment 300 or embodiment 301, wherein the trastuzumab is administered to the subject at a dose of about 4 mg/kg to about 10 mg/kg.
310. The use of embodiment 309, wherein the trastuzumab is administered to the subject at a dose of about 6 mg/kg.
311. The use of embodiment 309, wherein the trastuzumab is administered to the subject at a dose of about 8 mg/kg.
312. The use of embodiment 309, wherein the trastuzumab is administered to the subject at an initial dose of about 8 mg/kg followed by subsequent doses of about 6 mg/kg.
313. The use of any one of embodiments 309-312, wherein the trastuzumab is administered intravenously.
314. The use of any one of embodiments 306-313, wherein the trastuzumab is administered once about every 1 week, once about every 2 weeks, once about every 3 weeks, or once about every 4 weeks.
315. The use of embodiment 314, wherein the trastuzumab is administered once about every 3 weeks.
316. The use of embodiment 301, wherein the tucatinib, capecitabine and trastuzumab are administered to the subject on a 21 day treatment cycle.
317. The use of embodiment 316, wherein the tucatinib is administered to the subject twice per day on each day of the 21 day treatment cycle.
318. The use of embodiment 316 or 317, wherein the capecitabine is administered to the subject twice per day on each of days 1-14 of the 21 day treatment cycle.
319. The use of any one of embodiments 316-318, wherein the trastuzumab is administered to the subject once per 21 day treatment cycle.
320. The use of embodiment 319, wherein the dose of trastuzumab during the first 21 day treatment cycle is 8 mg/kg and the dose of trastuzumab during the subsequent 21 day treatment cycles is 6 mg/kg.
321. The use of any one of embodiments 225-320, wherein the subject has been previously treated with one or more additional therapeutic agents for the breast cancer.
322. The use of embodiment 321, wherein the one or more additional therapeutic agents is an anti-HER2 antibody or anti-HER2 antibody-drug conjugate.
323. The use of embodiment 322, wherein the one or more additional therapeutic agents is trastuzumab, pertuzumab and/or T-DM1.
324. The use of any one of embodiments 225-323, wherein the subject has not been treated with another therapeutic agent for the breast cancer within the past 12 months.
325. The use of any one of embodiments 225-320, wherein the subject has not previously been treated with another therapeutic agent for the breast cancer.
326. The use of any one of embodiments 225-325, wherein the subject has not previously been treated with lapatinib, neratinib, afatinib, or capecitabine.
327. The use of any one of embodiments 225-326, wherein treating the subject results in a tumor growth inhibition (TGI) index of at least about 85%.
328. The use of any one of embodiments 225-326, wherein treating the subject results in a TGI index of about 100%.
329. The use of any one of embodiments 225-328, wherein one or more therapeutic effects in the subject is improved after administration of tucatinib to the subject relative to a baseline.
330. The use of embodiment 329, wherein the one or more therapeutic effects is selected from the group consisting of: size of a tumor derived from the breast cancer, objective response rate, duration of response, time to response, progression free survival and overall survival.
331. The use of any one of embodiments 225-330, wherein the size of a tumor derived from the breast cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the breast cancer before administration of tucatinib to the subject.
332. The use of any one of embodiments 225-331, wherein the objective response rate is at least about 20%, at least about 25%, at least about 30%, at least about 35%, at least about 40%, at least about 45%, at least about 50%, at least about 60%, at least about 70%, or at least about 80%.
333. The use of any one of embodiments 225-332, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of tucatinib to the subject.
334. The use of any one of embodiments 225-333, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of tucatinib to the subject.
335. The use of any one of embodiments 225-334, wherein the duration of response to tucatinib is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of tucatinib to the subject.
336. The use of any one of embodiments 225-335, wherein the subject is a human.

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1: A Phase I Drug-Drug Interaction Study of Tucatinib in Healthy Subjects Receiving MATE1/2K Substrate

Tucatinib is a potent, selective, adenosine triphosphate (ATP)-competitive small molecule inhibitor of the receptor tyrosine kinase HER2. Tucatinib is investigated as single agent or in combination with capecitabine and trastuzumab in clinical trials for the treatment of patients with advanced solid tumors, including HER2+ breast cancer (see e.g., The HER2CLIMB clinical trial (ClinicalTrials.gov Identifier #NCT02614794)).

Drug-drug interactions may have significant impact on the efficacy and toxicity of therapeutic agents administered to cancer patients. In the proximal tubule, basic drugs are transported from the renal cells to the tubule lumen through the concerted action of the H+/organic cation antiporters, multidrug and toxin extrusion 1 (MATE1) and 2K (MATE2K). Inhibitors of MATE transporters have been shown to have a clinically relevant effect on the pharmacokinetics (PK) of concomitantly administered drugs such as metformin.

Metformin is a commonly used oral glucose-lowering drug for type 2 diabetes. The drug is also commonly used as in vivo OCT2/MATE1/MATE2K probe in drug-drug interaction (DDI) studies conducted during development of inhibitors of OCTs and/or MATEs.

In vitro assessment tucatinib inhibited the activity of organic cation transporter (OCT)2, breast cancer resistance protein (BCRP) and bile salt export pump (BSEP) with 50% inhibitory concentration (IC₅₀) values of 14.7 μM, 8.98 μM, and 8.48 μM, respectively. Tucatinib also inhibited MATE1 and MATE2K transporters in vitro with inhibition IC₅₀ values of 0.34 μM and 0.14 μM, respectively.

Tucatinib has the potential to affect PK of drugs affected by the OCT2/MATE1/MATE2K molecules. Given the importance of these pathways on drug metabolism it is important to understand how tucatinib acts on these molecules to more accurately understand its potential drug-drug interaction potential. This is important given the large number of treatments cancer patients maybe prescribed.

### Methods

A Phase 1, single center, open-label, fixed-sequence, drug-drug interaction (DDI) study was conducted to assess the effects tucatinib on the pharmacokinetics of metformin in healthy male and female subjects.

A primary objective of the study was to assess the effects of multiple bid oral doses of tucatinib on the single-dose PK of a substrate of multidrug and toxin extrusion protein (MATE)1/2K, metformin. Secondary objectives of the study included: assessing the safety and tolerability of metformin when co-administered with tucatinib; assessing the effects of tucatinib on renal function using iohexol as GFR marker; and assessing PK (e.g., trough PK profile) of multiple bid oral doses of tucatinib in the study subjects.

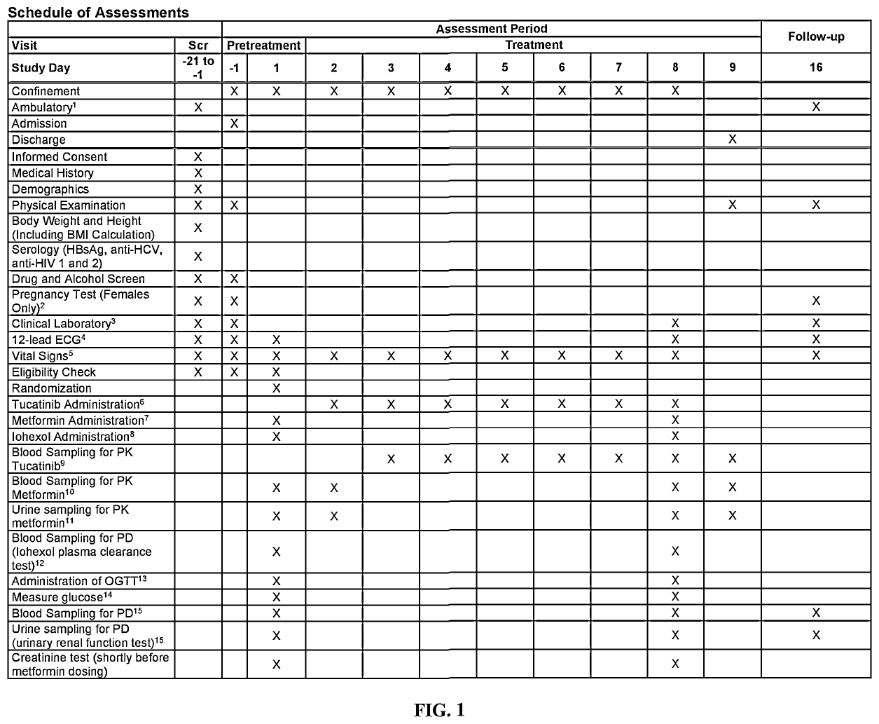

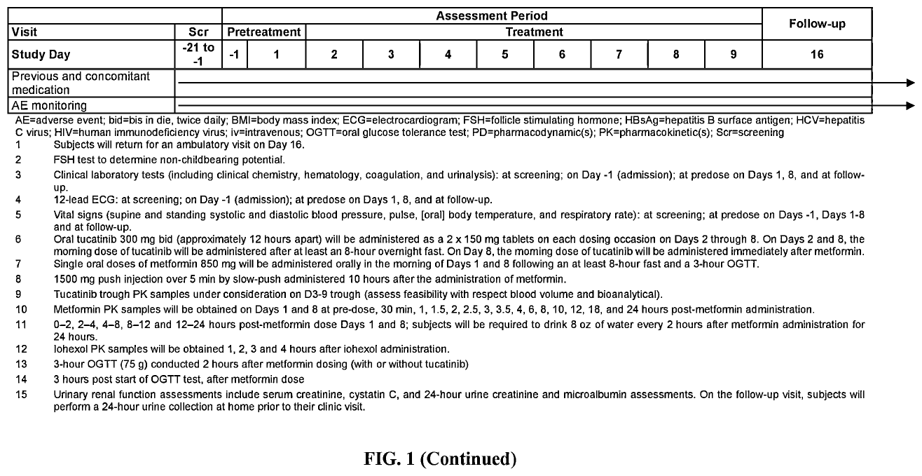

18 healthy subjects were enrolled to complete treatment and assessment as shown in FIG. 1. The subjects were admitted to the clinical research center in the afternoon of Day −1, which is the day prior to Day 1, the day of the first drug administration. They were discharged on Day 9 after completion of the in-patient assessments. After discharge, the subjects returned to the clinical research center for an out-patient follow-up visit on Day 16. Of the 18 subjects, 17 completed the study for evaluations of PK and PD profiles.

On Days 1 and 8, each subject received oral administration of 850 mg metformin in the morning after at least an overnight fast of ≥8 hours and a 3-hour oral glucose tolerance test. On Days 1 and 8, each subject also received 1500 mg iohexol (a contrast agent and GFR marker used for plasma clearance test) push injection over 5 minutes 10 hours after metformin administration. On Days 2-8, each subject received oral administration of 300 mg tucatinib, twice daily (bid) (approximately 12 hours apart) as 2×150 mg tablets. On Days 2 and 8, the morning dose of tucatinib was administered after an overnight fast of ≥8 hours. On Day 8, the morning dose of tucatinib was administered immediately after metformin dosing. Tucatinib and metformin were provided as oral tablets, and iohexol was provided as a solution at a suitable concentration.

Safety assessments and blood/urine sampling for pharmacokinetics/pharmacodynamics (PK/PD) determination were conducted from Day −1 to Day 9 according to the schedule shown in FIG. 1. PK endpoints included: plasma tucatinib (and metabolite ONT-993) trough concentrations, plasma and urine concentrations of metformin, plasma PK parameters for metformin estimated using noncompartmental analysis (NCA) (e.g., maximum plasma concentration (Cmax), time to maximum plasma concentration (tmax), half-life (t ½), area under the plasma concentration-time curve from time 0 to the last available measurement (AUC0-last), area under the plasma concentration-time curve from time 0 extrapolated to infinity (AUC0-inf), clearance (CL), apparent volume of distribution (Vz/F), and oral clearance (CL/F)), trough concentrations of tucatinib, and iohexol plasma clearance. PD endpoints were assessed by conducting a 3-hour oral glucose tolerance test (OGTT at 75 g) 2 hours after metformin dosing with or without tucatinib, and measuring serum levels of creatinine and cystatin C, as well as 24-hour urine creatinine and microalbumin levels. Safety endpoints included assessments of adverse events (AEs), clinical laboratory tests, measurements of vital signs, 12-lead electrocardiograms (ECG) and physical examinations.

Clinical laboratory tests measured the following parameters: (1) hematology, including erythrocytes (RBCs), leukocytes (WBCs) with differential (neutrophils, eosinophils, lymphocytes, monocytes, and basophils), hemoglobin, hematocrit, and platelet count; (2) blood chemistry, including sodium, potassium, chloride, bicarbonate, creatinine, creatine kinase (CK), amylase, lipase, glucose (fasting), urea, albumin, calcium, magnesium, inorganic phosphorus, alkaline phosphatase, ASAT (aspartate aminotransferase), ALAT (alanine aminotransferase), total bilirubin, indirect and direct bilirubin, total protein, total cholesterol, high-density lipoprotein (HDL), low-density lipoprotein (LDL), triglycerides, and uric acid; and (3) urinalysis: A midstream, clean-catch urine specimen was collected for dipstick analysis of protein, blood, glucose, WBCs, and pH (to be captured in the database). Additionally, urine was collected for the assessment of the following drugs of abuse: alcohol, cannabinoids, amphetamines, opiates, methadone, cocaine, cotinine, benzodiazepines, and barbiturates. Results were reviewed before dosing on Day 1. Serology was collected for the measurement of HIV-1 and -2 antibodies, hepatitis B surface antigen, and hepatitis C antibody. For females, a serum pregnancy test was collected and results were reviewed before dosing on Day 1.

Inclusion criteria for eligible subjects included the following: (1) Male or female of non-childbearing potential; (2) 18 to 65 years, inclusive, at screening; (3) Body mass index (BMI) of 18.0-32.0 kg/m², inclusive; (4) Body weight ≥60 kg; (5) Healthy status. Healthy status was defined by the absence of evidence of any clinically significant, active or chronic disease following a detailed medical and surgical history, a complete physical examination including vital signs, 12-lead ECG, hematology, blood chemistry, serology, and urinalysis; (6) Ability and willingness to abstain from alcohol-, caffeine-, and xanthine-containing beverages or food (e.g., coffee, tea, cola, chocolate, energy drinks) from 48 hours (2 days) prior to each admission to the clinical facility until study discharge (including clinic furloughs); (7) All values for hematology and clinical chemistry tests of blood and urine within the 1.5×the upper limit of normal range or showing no clinically relevant deviations; (8) Males who were sexually active with a woman of childbearing potential and were not surgically sterile for at least 90 days must have agreed to use a barrier method of birth control e.g., either condom or partner with occlusive cap (diaphragm or cervical/vault caps) with spermicidal foam/gel/film/cream/suppository for the duration of the study plus 3 months after receiving the last dose of study drug, and all men must not donate sperm during the study and for 3 months after receiving the last dose of study drug. In addition, their female partners should consider the use of an additional method of birth control (which may include a hormonal method, an intrauterine device [IUD] or an intrauterine system [IUS]) for at least the same duration; and (9) All non-regular medication (including over-the-counter medication, health supplements, and herbal remedies such as St. John's Wort extract) must have been stopped at least 28 days prior to admission to the clinical research center. An exception was made for paracetamol (acetaminophen), which was allowed up to admission to the clinical research center.

Exclusion criteria included the following: (1) Women of childbearing potential; (2) Women who were lactating; (3) Males with female partners who were pregnant, lactating, or planning to attempt to become pregnant during this study or within 90 days after dosing of study drug; (4) Use of any investigational drug or device within 30 days of the first dose of study medication; (5) Any disease or medical condition which poses an unacceptable risk to the subjects; (6) Any condition that may affect drug absorption (including stomach or intestinal surgery); (7) Significant history of metabolic, allergic, dermatological, hepatic, renal, hematological, pulmonary, cardiovascular, gastrointestinal, neurological, respiratory, endocrine, or psychiatric disorder; (8) History of hypersensitivity, intolerance, or allergy to any drug compounds, food, or other substance; (9) Using tobacco products within 28 days prior to admission; (10) Routine or chronic use of more than 3 grams of acetaminophen daily; (11) Strenuous activity, sunbathing, and contact sports within 48 hours (2 days) prior to (first) admission to the clinical facility and for the duration of the study; (12) Blood transfusion within 90 days of study drug administration; (13) Inability to be venipunctured and/or tolerate venous access; (14) Donation of blood to a blood bank or in a clinical study (except a screening visit) within 8 weeks of initial study drug administration; (15) History of donation of more than 450 mL of blood within 60 days prior to dosing in the clinical research center or planned donation before 30 days has elapsed since intake of study drug; (16) Plasma or platelet donation within 7 days of initial study drug administration; (17) History of alcoholism or drug abuse within 2 years; (18) History of alcohol consumption exceeding 7 standard drinks per week for female subjects or 14 drinks/week for male subjects. Alcohol consumption was prohibited 48 hours prior to admission to the clinical facility and throughout the entire study until discharge; (19) Use or intent to use any prescription medications within 28 days prior to initial dose of study treatment; (20) Positive screening test for hepatitis B surface antigen (HBsAg), antihepatitis C virus (HCV) antibodies, or antihuman immunodeficiency virus (HIV) 1 and 2 antibodies; (21) Acute or chronic metabolic acidosis, including diabetic ketoacidosis, with or without coma; and (22) Renal disease or renal dysfunction (e.g., as suggested by serum creatinine levels ≥1.5 mg/dL [males], ≥1.4 mg/dL [females] or abnormal creatinine clearance).

### Results

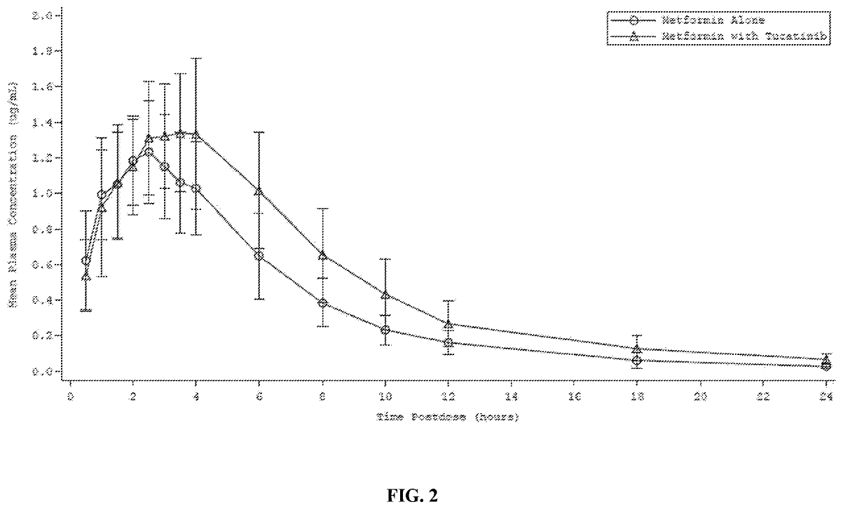

As shown in FIG. 2, metformin Cₘₐₓ plasma concentrations were higher following administration of metformin in combination with tucatinib at steady state on Day 8 than those on Day 1 following metformin administration alone, with similarly shaped profiles following both treatments.

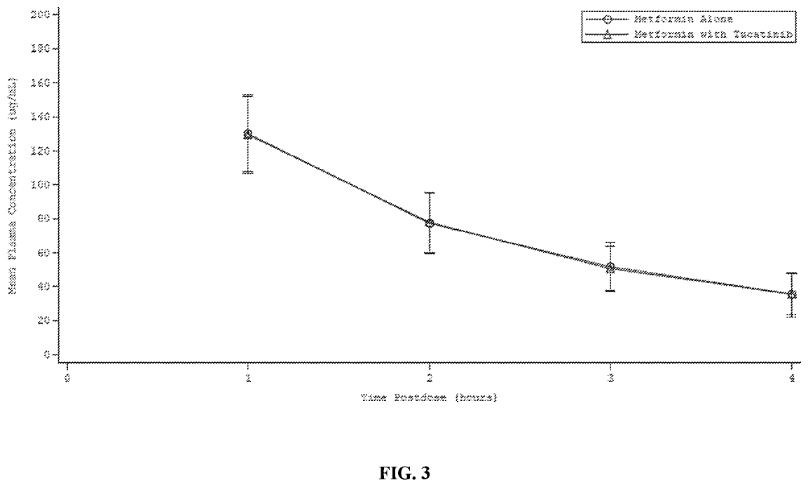

As shown in FIG. 3, iohexol postdose plasma concentrations were above the lower limit of quantitation (LLOQ: 5 μg/mL) in all subjects through 4 hours postdose on Day 1 and Day 8, with similar mean iohexol plasma concentrations.

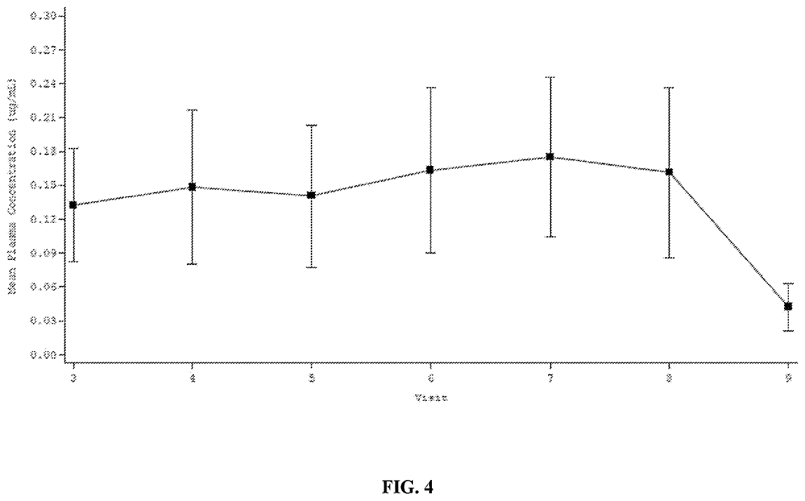

As shown in FIG. 4, steady-state plasma concentrations of tucatinib were reached by Day 6.

### Pharmacokinetic Parameters

Iohexol

Mean iohexol PK parameters were similar following coadministration of tucatinib with metformin compared to metformin alone. The aGFR estimated using the Jødal and Brøchner Mortensen equation remained constant (94.99 mL/min/1.73 m² for metformin alone compared with 94.56 mL/min/1.73 m² for metformin plus tucatinib).

Metformin

Following coadministration of tucatinib with metformin, arithmetic mean metformin Cₘₐₓ, AUC₀₋ₗₐₛₜ, and AUC_{0-inf} increased 1.102-, 1.376-, and 1.409-fold, respectively, compared to metformin alone. The variability (% CV) in exposure was generally low for both treatments (ranged from 18.0% to 30.5%). Metformin median Tₘₐₓ was slightly delayed following administration in combination with tucatinib at steady state (3.000 hours) compared to metformin alone (2.500 hours). Though metformin Tₘₐₓ ranges were similar (1.50 to 4.00 hours for metformin alone; 1.00 to 4.07 hours for combination), most subjects showed a delay in Tₘₐₓ following combination therapy. The mean terminal elimination half-life (t_{1/2}) appeared slightly longer following combination treatment (5.569 hours) compared to metformin alone (4.546 hours). The mean CL/F decreased from 105.4 L/h to 77.4 L/h in the presence of tucatinib, while the mean apparent volume of distribution (Vz/F) decreased slightly from 695.4 L to 627.2 L (Table 1).

TABLE 1
Summary Statistics for Plasma Metformin Pharmacokinetic Parameters

| | Arithmetic Mean (CV %) | |
|---|---|---|
| | Metformin Alone | Metformin + Tucatinib |
| Pharmacokinetic Parameters | (N = 17) | (N = 17) |
| Cₘₐₓ (μg/mL) | 1.334 (18.0) | 1.470 (27.9) |
| Tₘₐₓ (h) ^{a} | 2.500 (1.50, 4.00) | 3.000 (1.00, 4.07) |
| AUC₀₋ₗₐₛₜ (h*μg/mL) | 8.415 (26.2) | 11.582 (29.9) |
| AUC_{0-inf} (h*μg/mL) | 8.608 (26.4) | 12.129 (30.5) |
| t_{1/2} (h) | 4.546 (11.7) | 5.569 (12.2) |
| V_{z/F} (L) | 695.4 (32.6) | 627.2 (41.5) |
| CL/F (L/h) | 105.4 (26.7) | 77.4 (34.9) |

^{a} For Tₘₐₓ the median (range) is presented instead of arithmetic mean (CV %).

### Statistical Analysis of Pharmacokinetic Parameters

Coadministration with tucatinib had no apparent effect on plasma metformin Cₘₐₓ, as the geometric LS mean of metformin following coadministration with tucatinib (Test) relative to that of metformin alone (Reference) was close to 1, and the 90% CI was contained within the standard no-effect boundary of (0.80, 1.25). Metformin coadministration with tucatinib led to an approximately 1.357- and 1.387-fold higher plasma metformin AUC₀₋ₗₐₛₜ and AUC_{0-inf}, respectively (Table 2), with the 90% CIs outside the standard no-effect boundary.

TABLE 2
Statistical Analysis of the Effect of Tucatinib on Metformin Pharmacokinetic
Parameters

| | Geometric Least-Squares (LS) Mean | | | | | |
|---|---|---|---|---|---|---|
| | Metformin with | | Metformin Alone | | Geometric LS Mean Ratio | |
| | Tucatinib | | (Reference) | | (Test/Reference) | |
| Parameter | n | Result | n | Result | Estimate | 90% Cl |
| AUC₀₋ₗₐₛₜ (h* μg/mL) | 17 | 11.054 | 17 | 8.147 | 1.357 | (1.220, 1.509) |
| AUC₀₋ᵢₙₜ (h* μg/mL) | 17 | 11.558 | 17 | 8.333 | 1.387 | (1.251, 1.539) |
| Cₘₐₓ (μg/mL) | 17 | 1.418 | 17 | 1.314 | 1.079 | (0.951, 1.225) |

### Conclusions

A statistically significant increase in metformin exposure and reductions in CL/F and CLᵣₑₙₐₗ were observed when metformin was co-administered with tucatinib, consistent with tucatinib inhibiting the renal secretion of metformin. Additionally, an increase in mean serum creatinine was observed following multiple doses of tucatinib; however, measures of renal function including aGFR as assessed by iohexol and urine albumin levels were not affected. These results are consistent with tucatinib acting as a weak inhibitor of the renal OCT2/MATE1/MATE2-K pathway and indicate tucatinib does not cause renal damage.

### Example 2: A Phase 1 Drug-Drug Interaction Study of Tucatinib in Healthy Subjects Receiving Substrates of CYP3A4, CYP2C8, CYP2C9 and P-Glycoprotein

Drug-drug interactions of tucatinib with substrates of CYP3A4, CYP2C8, CYP2C9 and P-glycoprotein were evaluated. Tucatinib was predicted to have good-to-moderate stability with respect to hepatic metabolism across species, and was predicted to be metabolized in human liver primarily by cytochrome P450 (CYP)2C8 to yield the metabolite, ONT-993. Tucatinib inhibits CYP2C8, CYP2C9, and CYP3A4 in vitro with Ki values of 0.17 μM, 4.57 μM, and 0.81 μM, respectively, but no time-dependent inhibition of CYP3A4 was observed. Mean clinical maximum observed concentration (Cmax) of tucatinib is approximately 1 to 2 μM; therefore, the risk of inhibition of CYP2C8, CYP2C9, and CYP3A4 by tucatinib at clinically relevant drug levels is possible. Tucatinib did not induce in vitro enzyme activity or messenger ribonucleic acid associated with CYP3A4 or CYP1A2 in human hepatocytes. Additionally, tucatinib was found to be a P-glycoprotein (P-gp) substrate and a weak inhibitor (half maximal inhibitory concentration [IC50] approximately 10 to 30 μM) of P-gp-mediated efflux of digoxin.

### Methods

A Phase 1, open-label, fixed-sequence, 5-part, drug-drug interaction study of tucatinib was conducted to evaluate the effects of CYP3A4 and CYP2C8 inhibition and induction on the pharmacokinetics of tucatinib and to evaluate the effects of tucatinib on the pharmacokinetics of substrates of CYP3A4, CYP2C8, CYP2C9, and P-glycoprotein in healthy male and female subjects.

A total of 116 patients were enrolled into five different groups: Part A, Part B, Part C, Part D, and Part E. Part A evaluates the effect of the strong CYP3A4 inhibitor itraconazole on the PK of tucatinib. Part B evaluates the effect of rifampin, a strong inducer of CYP3A4 and CYP2C8, on the PK of tucatinib. Part C evaluates the effect of the strong CYP2C8 inhibitor gemfibrozil on the PK of tucatinib. Part D evaluates the effects of tucatinib on the PK of substrate probes of the metabolizing enzymes CYP2C8 (repaglinide), CYP2C9 (tolbutamide), and CYP3A4 (midazolam). Part E evaluates the effect of tucatinib on the PK of a substrate probe of the transporter P-gp (digoxin). Parts A, B, C, D, and E of the study are independent of one another and do not need to be conducted in any particular order. For Parts A, B, and C, tucatinib is given as a single oral dose because evaluating plasma exposures after a single dose of tucatinib is expected to provide an appropriate assessment of the impact of the probe drugs on tucatinib PK. In Parts D and E, tucatinib is given as a multiple-dosing regimen in order to examine its effects on probe drugs at steady state, at which maximal inhibition of CYP2C8, CYP2C9, CYP3A4, and P-gp by tucatinib should be achieved.

### Part A

A primary objective of the study is to assess the effect of a strong CYP3A4 inhibitor (itraconazole) on the single-dose PK of tucatinib. A secondary objective of the study is to assess the safety and tolerability of tucatinib when administered alone and when co-administered with a strong CYP3A4 inhibitor. Exploratory objectives of the study included: (1) assessing the effect of a strong CYP3A4 inhibitor on the PK of ONT-993, a metabolite of tucatinib, following a single dose of tucatinib; and (2) evaluating the potential effect of genetic CYP polymorphisms or other genetic polymorphisms on any observed variable response in the magnitude of the drug interaction between tucatinib and any probe drugs or substrates.

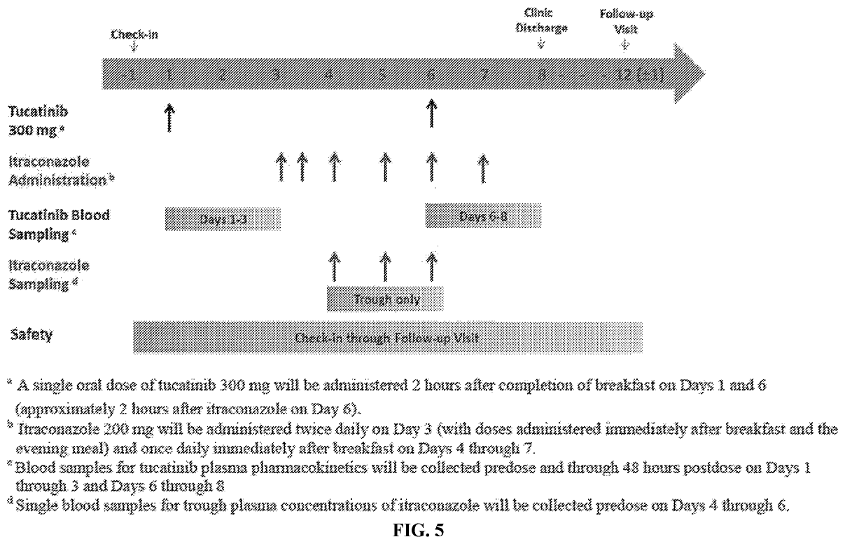

28 healthy subjects were enrolled to complete treatment and assessment as shown in FIG. 5. The subjects were admitted to the clinical research center in the afternoon of Day −1, which is the day prior to Day 1, the day of the first drug administration. They were discharged on Day 8 after completion of the in-patient assessments. After discharge, the subjects returned to the clinical research center for an out-patient follow-up visit on Day 11, 12 or 13.

On Day 1, each subject received a single oral dose of 300 mg of tucatinib at about 2 hours after the completion of breakfast in the morning. On Day 3, each subject received oral doses of 200 mg of itraconazole, twice daily (BID). From Day 4 to Day 7, each subject received single oral doses of 200 mg of itraconazole once daily (QD). Itraconazole was administered in the fed state (within 5 minutes after completion of meals). On Day 6, each subject received a single oral dose of 300 mg of tucatinib at about 2 hours after the completion of breakfast and after itraconazole in the morning. Tucatinib was provided as 150-mg tablets (2 tablets for the 300-mg dose), and itraconazole was provided as 100-mg capsules (2 capsules for the 200-mg dose).

To assess PK endpoints, blood samples were collected for the analysis of plasma concentrations of tucatinib and ONT-993 according to the schedule in FIG. 5. The following PK parameters were calculated, when possible, using standard noncompartmental methods: area under the concentration-time curve (AUC) from time 0 to infinity (AUC0-∞), AUC from time 0 to the time of the last quantifiable concentration (AUClast), percentage extrapolation in AUC (% AUCextrap), maximum observed concentration (Cmax), time of maximum observed concentration (Tₘₐₓ), apparent terminal elimination half-life (t½), apparent total clearance (CL/F; tucatinib only), apparent volume of distribution (Vz/F; tucatinib only), and metabolite-to-parent ratio based on AUC (MR_{AUC}; ONT-993 only). Other noncompartmental PK parameters may be reported. Additionally, blood samples were collected for determination of trough levels of itraconazole prior to morning dosing on the days indicated in FIG. 5. A single genotyping blood sample was collected to assess possible effect of CYP polymorphisms or other genetic polymorphisms on the magnitude of drug interactions of tucatinib with probe drugs and substrates.

Safety endpoints of the study were assessed by monitoring adverse events (AEs), clinical laboratory evaluations (clinical chemistry, hematology, and urinalysis), vital signs measurements, 12-lead electrocardiograms (ECGs), and physical examinations.

### Part B

A primary objective of the study was to assess the effect of an inducer of CYP3A4 and CYP2C8 (rifampin) on the single-dose PK of tucatinib. A secondary objective of the study was to assess the safety and tolerability of tucatinib when administered alone and when co-administered with a strong inducer of CYP3A4 and CYP2C8. Exploratory objectives of the study included: (1) assessing the effect of an inducer of CYP3A4 and CYP2C8 on the PK of ONT-993 following a single dose of tucatinib; and (2) evaluating the potential effect of genetic CYP polymorphisms or other genetic polymorphisms on any observed variable response in the magnitude of the drug interaction between tucatinib and any probe drugs or substrates.

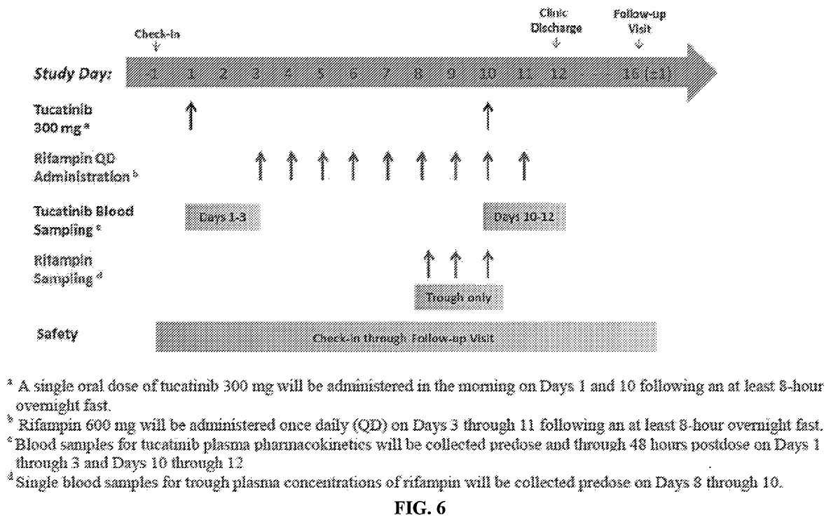

28 healthy subjects were enrolled to complete treatment and assessment as shown in FIG. 6. The subjects were admitted to the clinical research center in the afternoon of Day −1, which is the day prior to Day 1, the day of the first drug administration. They were discharged on Day 12 after completion of the in-patient assessments. After discharge, the subjects returned to the clinical research center for an out-patient follow-up visit on Day 15, 16 or 17.

On Day 1, each subject received a single oral dose of 300 mg of tucatinib. On Day 3 through Day 11, each subject received single oral doses of 600 mg of rifampin once daily (QD). On Day 10, each subject received a single oral dose of 300 mg of tucatinib. Tucatinib was provided as 150-mg tablets (2 tablets for the 300-mg dose), and rifampin was provided as 300-mg capsules (2 capsules for the 600-mg dose). Tucatinib and rifampin were administered before meals preceded by an at least 8-hour overnight fast.

To assess PK endpoints, blood samples were collected for the analysis of plasma concentrations of tucatinib and ONT-993 according to the schedule in FIG. 6. The following PK parameters were calculated, when possible, using standard noncompartmental methods: area under the concentration-time curve (AUC) from time 0 to infinity (AUC0-∞), AUC from time 0 to the time of the last quantifiable concentration (AUClast), percentage extrapolation in AUC (% AUCextrap), maximum observed concentration (Cmax), time of maximum observed concentration (Tmax), apparent terminal elimination half-life (t½), apparent total clearance (CL/F; tucatinib only), apparent volume of distribution (Vz/F; tucatinib only), and metabolite-to-parent ratio based on AUC (MR_{AUC}; ONT-993 only). Other noncompartmental PK parameters may be reported. Additionally, blood samples were collected for determination of trough levels of rifampin prior to morning dosing on the days indicated in FIG. 6. A single genotyping blood sample was collected to assess possible effect of CYP polymorphisms or other genetic polymorphisms on the magnitude of drug interactions of tucatinib with probe drugs and substrates.

Safety endpoints of the study were assessed by monitoring adverse events (AEs), clinical laboratory evaluations (clinical chemistry, hematology, and urinalysis), vital signs measurements, 12-lead electrocardiograms (ECGs), and physical examinations.

### Part C

A primary objective of the study was to assess the effect of a strong CYP2C8 inhibitor (gemfibrozil) on the single-dose PK of tucatinib. A secondary objective of the study was to assess the safety and tolerability of tucatinib when administered alone and when co-administered with a strong CYP2C8 inhibitor. Exploratory objectives of the study included: (1) assessing the effect of a strong CYP2C8 inhibitor on the PK of ONT-993 following a single dose of tucatinib; and (2) evaluating the potential effect of genetic CYP polymorphisms or other genetic polymorphisms on any observed variable response in the magnitude of the drug interaction between tucatinib and any probe drugs or substrates.

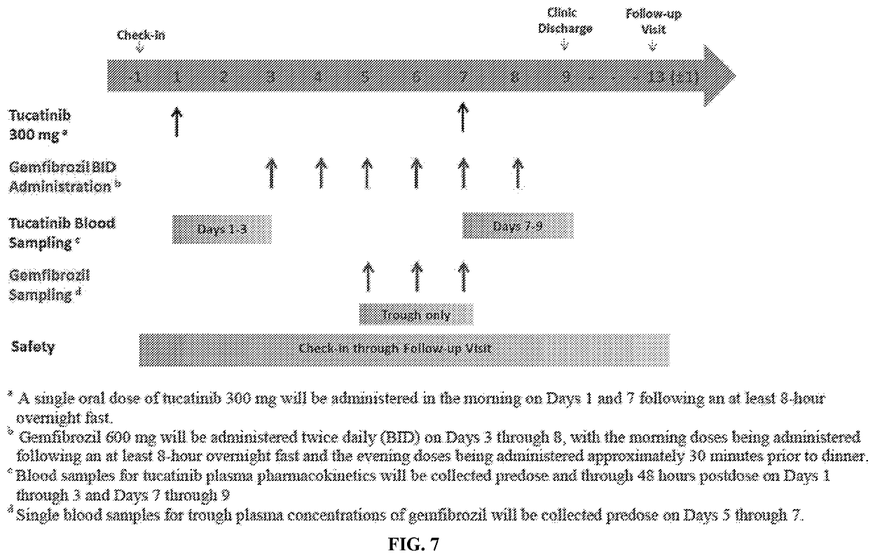

28 healthy subjects were enrolled to complete treatment and assessment as shown in FIG. 7. The subjects were admitted to the clinical research center in the afternoon of Day −1, which is the day prior to Day 1, the day of the first drug administration. They were discharged on Day 9 after completion of the in-patient assessments. After discharge, the subjects return to the clinical research center for an out-patient follow-up visit on Day 12, 13 or 14.

On Day 1, each subject received a single oral dose of 300 mg of tucatinib. From Day 3 to Day 8, each subject received single oral doses of 600 mg of gemfibrozil (BID), twice daily (BID). On Day 7, each subject received a single oral dose of 300 mg of tucatinib. Tucatinib was provided as 150-mg tablets (2 tablets for the 300-mg dose), and gemfibrozil was provided as 600-mg tablets. Tucatinib and gemfibrozil were administered before meals preceded by an at least 8-hour overnight fast.

To assess PK endpoints, blood samples were collected for the analysis of plasma concentrations of tucatinib and ONT-993 according to the schedule in FIG. 7. The following PK parameters were calculated, when possible, using standard noncompartmental methods: area under the concentration-time curve (AUC) from time 0 to infinity (AUC0-∞), AUC from time 0 to the time of the last quantifiable concentration (AUClast), percentage extrapolation in AUC (% AUCextrap), maximum observed concentration (Cmax), time of maximum observed concentration (Tmax), apparent terminal elimination half-life (t½), apparent total clearance (CL/F; tucatinib only), apparent volume of distribution (Vz/F; tucatinib only), and metabolite-to-parent ratio based on AUC (MR_{AUC}; ONT-993 only). Other noncompartmental PK parameters may be reported. Additionally, blood samples were collected for determination of trough levels of gemfibrozil prior to morning dosing on the days indicated in FIG. 7. A single genotyping blood sample was collected to assess possible effect of CYP polymorphisms or other genetic polymorphisms on the magnitude of drug interactions of tucatinib with probe drugs and substrates.

Safety endpoints of the study were assessed by monitoring adverse events (AEs), clinical laboratory evaluations (clinical chemistry, hematology, and urinalysis), vital signs measurements, 12-lead electrocardiograms (ECGs), and physical examinations.

### Part D

A primary objective of the study was to assess the effects of tucatinib on the single-dose PK of substrate probes of CYP2C8 (repaglinide), CYP2C9 (tolbutamide), and CYP3A4 (midazolam). Secondary objectives of the study included: (1) assessing the safety and tolerability of tucatinib when administered alone and when co-administered with substrate probes of CYP2C8, CYP2C9, and CYP3A4; and (2) assessing the single-dose PK of tucatinib and assessing the multiple-dose, steady-state PK of tucatinib alone and in the presence of substrate probes of CYP2C8, CYP2C9, and CYP3A4 in healthy subjects. Exploratory objectives of the study included: (1) assessing the effects of tucatinib on the PK of relevant metabolites of tolbutamide (4-hydroxytolbutamide) and midazolam (1-hydroxymidazolam); and (2) evaluating the potential effect of genetic CYP polymorphisms or other genetic polymorphisms on any observed variable response in the magnitude of the drug interaction between tucatinib and any probe drugs or substrates.

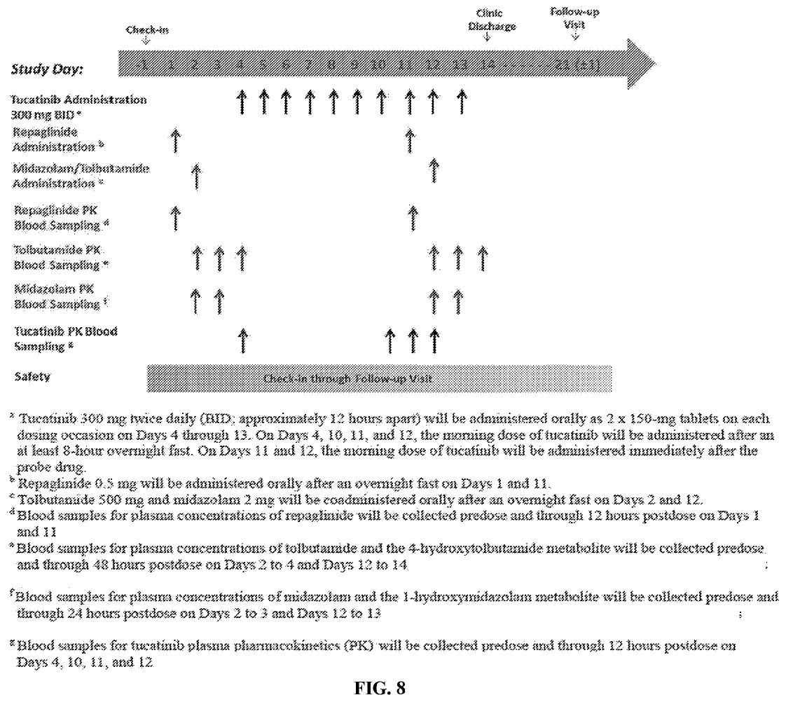

17 healthy subjects were enrolled to complete treatment and assessment as shown in FIG. 8. The subjects were admitted to the clinical research center in the afternoon of Day −1, which is the day prior to Day 1, the day of the first drug administration. They were discharged on Day 14 after completion of the in-patient assessments. After discharge, the subjects returned to the clinical research center for an out-patient follow-up visit on Day 20, 21 or 22.

On Day 1, each subject received a single oral dose of 0.5 mg of repaglinide. On Day 2, each subject received a single oral dose of 2 mg of midazolam and a single oral dose of 500 mg of tolbutamide, administered together. From Day 4 to Day 13, each subject received oral doses of 300 mg of tucatinib, twice daily (BID). On Day 11, each subject received a single oral dose of repaglinide. On Day 12, each subject received a single oral dose of 2 mg of midazolam and a single oral dose of 500 mg of tolbutamide, administered together. Tucatinib was provided as 150-mg tablets (2 tablets for the 300-mg dose), repaglinide was provided as 0.5-mg tablets, olbutamide was provided as 500-mg tablets, and midazolam was provided as a syrup (2 mg/mL; 1 mL syrup for the 2-mg dose). Tucatinib, repaglinide, olbutamide and midazolam were administered before meals preceded by an at least 8-hour overnight fast.

To assess PK endpoints, blood samples were collected for the analysis of plasma concentrations of repaglinide; tolbutamide and its 4-hydroxytolbutamide metabolite; and midazolam and its 1-hydroxymidazolam metabolite according to the schedule in FIG. 8. The following PK parameters were calculated, when possible, using standard noncompartmental methods: AUC0-∞, AUClast, % AUCextrap, Cmax, Tmax, t½, CL/F (repaglinide, tolbutamide, and midazolam only), Vz/F (repaglinide, tolbutamide, and midazolam only), and MR_{AUC} (4-hydroxytolbutamide and 1-hydromidazolam only). Other noncompartmental PK parameters may be reported. Additionally, blood samples for determination of plasma concentrations of tucatinib and ONT-993 were collected at the time points indicated in FIG. 8. The following PK parameters were calculated, when possible, using standard noncompartmental methods: AUC0-∞ (Days 4 and 8), AUClast, % AUCextrap, AUC within a dosing interval (AUCtau), Cmax, Tmax, t½, CL/F (Days 4 and 8, tucatinib only), apparent total clearance at steady state (CLss/F; Days 10, 11, 12, 14, and 15, tucatinib only), Vz/F (Days 4 and 8, tucatinib only), apparent volume of distribution at steady state (Vss/F; Days 10, 11, 12, 14, and 15, tucatinib only), accumulation ratio (Rac; Days 10, 11, 12, 14, and 15 only), and MR_{AUC} (ONT-993 only). A single genotyping blood sample was collected to assess possible effect of CYP polymorphisms or other genetic polymorphisms on the magnitude of drug interactions of tucatinib with probe drugs and substrates.

Safety endpoints of the study were assessed by monitoring adverse events (AEs), clinical laboratory evaluations (clinical chemistry, hematology, and urinalysis), vital signs measurements, 12-lead electrocardiograms (ECGs), and physical examinations.

### Part E

A primary objective of the study was to assess the effect of tucatinib on the single-dose PK of a substrate probe of P-gp (digoxin). Secondary objectives of the study included: (1) assessing the safety and tolerability of tucatinib when administered alone and when co-administered with a substrate probe of P-gp; and (2) assessing the single-dose PK of tucatinib and assessing the multiple-dose, steady-state PK of tucatinib alone and in the presence of a substrate probe of P-gp in healthy subjects. An exploratory objective of the study was to evaluate the potential effect of genetic CYP polymorphisms or other genetic polymorphisms on any observed variable response in the magnitude of the drug interaction between tucatinib and any probe drugs or substrates.

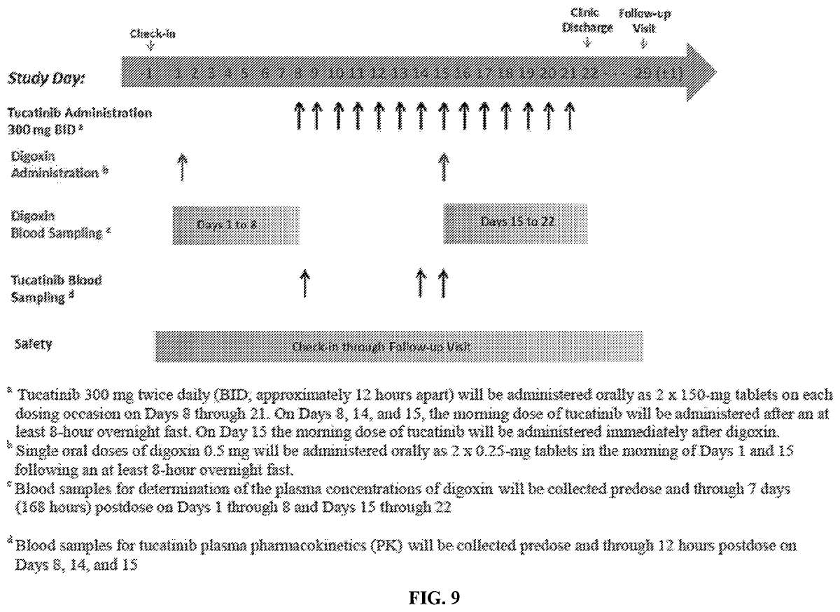

15 healthy subjects were enrolled to complete treatment and assessment as shown in FIG. 9. The subjects were admitted to the clinical research center in the afternoon of Day −1, which is the day prior to Day 1, the day of the first drug administration. They were discharged on Day 22 after completion of the in-patient assessments. After discharge, the subjects returned to the clinical research center for an out-patient follow-up visit on Day 28, 29 or 30.

On Day 1, each subject received a single oral dose of 0.5 mg of digoxin. From Day 8 to Day 21, each subject received oral doses of 300 mg of tucatinib, twice daily (BID). On Day 15, each subject received a single oral dose of 0.5 mg of digoxin. Tucatinib was provided as 150-mg tablets (2 tablets for the 300-mg dose), and digoxin was provided as 0.25-mg tablets (2 tablets for the 0.5-mg dose). Tucatinib and digoxin were administered before meals preceded by an at least 8-hour overnight fast.

To assess PK endpoints, blood samples were collected for the analysis of plasma concentrations of digoxin according to the schedule in FIG. 9. The following PK parameters were calculated, when possible, using standard noncompartmental methods: AUC0-∞, AUClast, % AUCextrap, Cmax, Tmax, t½, CL/F, and Vz/F. Other noncompartmental PK parameters may be reported. Additionally, blood samples for determination of plasma concentrations of tucatinib and ONT-993 were collected at the time points indicated in FIG. 9. The following PK parameters were calculated, when possible, using standard noncompartmental methods: AUC0-∞ (Days 4 and 8), AUClast, % AUCextrap, AUC within a dosing interval (AUCtau), Cmax, Tmax, t½, CL/F (Days 4 and 8, tucatinib only), apparent total clearance at steady state (CLss/F; Days 10, 11, 12, 14, and 15, tucatinib only), Vz/F (Days 4 and 8, tucatinib only), apparent volume of distribution at steady state (Vss/F; Days 10, 11, 12, 14, and 15, tucatinib only), accumulation ratio (Rac; Days 10, 11, 12, 14, and 15 only), and MR_{AUC} (ONT-993 only). A single genotyping blood sample was collected to assess possible effect of CYP polymorphisms or other genetic polymorphisms on the magnitude of drug interactions of tucatinib with probe drugs and substrates.

Safety endpoints of the study were assessed by monitoring adverse events (AEs), clinical laboratory evaluations (clinical chemistry, hematology, and urinalysis), vital signs measurements, 12-lead electrocardiograms (ECGs), and physical examinations.

Inclusion criteria for eligible subjects in Parts A-E included the following: (1) Males and females between 18 and 65 years of age, inclusive, at Screening; (2) Body mass index between 18.0 and 32.0 kg/m2, inclusive, and a total body weight between 50.0 and 100.0 kg, inclusive, at Screening; (3) Female subjects participating in the study were of non-childbearing potential and were therefore not be required to use contraception. Male subjects were surgically sterile for at least 90 days or when sexually active with female partners of childbearing potential agreed to use contraception; and (4) Able to comprehend and willing to sign an ICF and to abide by the study restrictions.

Exclusion criteria for Parts A-E included the following: (1) Significant history or clinical manifestation of any metabolic, allergic, dermatological, hepatic, renal, hematological, pulmonary, cardiovascular, gastrointestinal, neurological, respiratory, endocrine, or psychiatric disorder; (2) Any condition possibly affecting drug absorption (e.g., gastrectomy, gastric banding, gastric bypass); (3) History of significant hypersensitivity, intolerance, or allergy to any drug compound, food, or other substance; (4) History of stomach or intestinal surgery or resection that would potentially alter absorption and/or excretion of orally administered drugs (uncomplicated appendectomy, cholecystectomy, and hernia repair will be allowed); (5) History of hyperbilirubinemia (e.g., Gilbert's syndrome); (6) History of alcoholism or drug/chemical abuse within 2 years prior to Check-in; (7) History of regular alcohol consumption exceeding 7 drinks/week for female subjects or 14 drinks/week for male subjects. One unit of alcohol equals 12 oz (360 mL) of beer, 11% oz (45 mL) of liquor, or 5 oz (150 mL) of wine; (8) Positive urine drug screen (including cotinine) at Screening or Check-in, or a positive alcohol breath test at Check-in; (9) Positive hepatitis panel and/or positive human immunodeficiency virus test; (10) Screening liver function tests (alanine aminotransferase, aspartate aminotransferase, and total bilirubin), serum creatinine, hemoglobin, or hematocrit values outside of the normal reference range; (11) For subjects participating in Part E, potassium or magnesium levels outside of the normal ranges at Screening or Check-in; (12) Single 12-lead ECG demonstrating QT interval corrected for heart rate using Fridericia's formula (QTcF) >450 msec for males and >470 msec for females at Screening or Check-in (may repeat twice at Screening and/or Check-in and average all 3 ECG QTcF values for inclusion/exclusion purposes), or history/evidence of long QT syndrome; (13) Participation in a clinical study involving administration of an investigational drug (new chemical entity) in the past 30 days or within 5 half-lives (whichever is longer) prior to Check-in; (14) Used or intended to use any medications/products known to alter drug absorption, metabolism, or elimination processes, including St. John's wort and known strong inhibitors or inducers of CYP3A4 or CYP2C8, within 30 days prior to Check-in and during the study, with the exception that medications stipulated as study drugs in the protocol will be used during the study; (15) Used or intended to use any prescription medications/products within 28 days prior to Check-in; (16) Used or intended to use any nonprescription medications/products (excluding paracetamol/acetaminophen) including vitamins, minerals, and phytotherapeutic/herbal/plant-derived preparations within 14 days prior to Check-in; (17) Use of tobacco- or nicotine-containing products within 3 months prior to Check-in; (18) Consumption of foods or beverages containing poppy seeds, grapefruit, or Seville oranges within 7 days prior to Check-in; (19) Consumption of caffeine-containing foods or beverages within 48 hours prior to Check-in; (20) Consumption of alcohol within 48 hours prior to Check-in; (21) Receipt of blood products within 2 months prior to Check-in; (22) Donation of blood from 56 days prior to Screening, plasma from 2 weeks prior to Screening, or platelets from 6 weeks prior to Screening; (23) Poor peripheral venous access; and (24) Have previously completed or withdrawn from this study or any other study investigating tucatinib, and have previously received the investigational product.

### Pharmacokinetic Results

Part A: Tucatinib/Intraconazole

The strong CYP3A4 inhibitor itraconazole increased the plasma exposure of tucatinib; the geometric LS mean ratios (90% CIs) for AUCₗₐₛₜ, AUC_{0-∞}, and Cₘₐₓ for tucatinib dosed in combination with itraconazole compared to tucatinib dosed alone were 1.33 (1.25, 1.41), 1.34 (1.26, 1.43), and 1.32 (1.23, 1.42), respectively. This effect was statistically significant based on 90% CI values.

Itraconazole increased the plasma exposure of ONT-993; the geometric LS mean ratios (90% CIs) for AUCₗₐₛₜ, AUC_{0-∞}, and Cₘₐₓ for ONT-993 following administration of tucatinib in combination with itraconazole compared to following administration of tucatinib alone were 1.58 (1.49, 1.68), 1.58 (1.49, 1.67), and 2.03 (1.89, 2.18), respectively. These effects were statistically significant based on 90% CI values.

The geometric mean MR_{AUC0-∞} and MR_{Cmax} values of ONT-993 were similar or slightly higher following administration of tucatinib in combination with itraconazole compared to following administration of tucatinib alone.

CYP3A4 has a minor role in tucatinib metabolism with little or no role in the formation of ONT-993.

Part B: Tucatinib/Rifampin

The strong CYP inducer rifampin decreased the plasma exposure of tucatinib; the geometric LS mean ratios (90% CIs) for AUCₗₐₛₜ, AUC_{0-∞}, and Cₘₐₓ for tucatinib dosed in combination with rifampin compared to tucatinib dosed alone were 0.517 (0.449, 0.596), 0.520 (0.452, 0.597), and 0.632 (0.531, 0.753), respectively. This effect was statistically significant based on 90% CI values.

Rifampin decreased the plasma exposure of ONT-993 based on AUCₗₐₛₜ and AUC_{0-∞} and increased the Cₘₐₓ of ONT-993. The geometric LS mean ratios (90% CIs) for AUCₗₐₛₜ, AUC_{0-∞}, and Cₘₐₓ for ONT-993 following administration of tucatinib in combination with rifampin compared to following administration of tucatinib alone were 0.750 (0.636, 0.884), 0.748 (0.640, 0.873), and 2.08 (1.70, 2.55), respectively. These effects were statistically significant based on 90% CI values.

The geometric mean MR_{AUC0-∞} and MR_{Cmax} values of ONT-993 were approximately 1.4-fold and 3.3-fold higher, respectively, following administration of tucatinib in combination with rifampin compared to following administration of tucatinib alone.

Rifampin likely induced enzymes (such as CYP2C8 and CYP3A4) involved in tucatinib metabolism.

Part C: Tucatinib/Gemfibrozil

The strong CYP2C8 inhibitor gemfibrozil increased the plasma exposure of tucatinib; the geometric LS mean ratios (90% CIs) for AUCₗₐₛₜ, AUC_{0-∞}, and Cₘₐₓ for tucatinib dosed in combination with gemfibrozil compared to tucatinib dosed alone were 2.99 (2.62, 3.41), 3.04 (2.66, 3.46), and 1.62 (1.47, 1.79), respectively. These effects were statistically significant based on 90% CI values.

Gemfibrozil decreased the plasma exposure of ONT-993; the geometric LS mean ratios (90% CIs) for AUCₗₐₛₜ, AUC0-∞, and Cₘₐₓ for ONT-993 following administration of tucatinib in combination with gemfibrozil compared to following administration of tucatinib alone were 0.767 (0.686, 0.858), 0.887 (0.801, 0.982), and 0.304 (0.263, 0.352), respectively. These effects were statistically significant based on 90% CI values.

The geometric mean MR_{AUC0-∞} and MR_{Cmax} values of ONT-993 were decreased by approximately 71% and 81%, respectively, following administration of tucatinib in combination with gemfibrozil compared to following administration of tucatinib alone.

CYP2C8 plays a role in tucatinib metabolism and in the formation of ONT-993.

Part D: Tucatinib/Repaglinide/Tolbutamide/Midazolam

Tucatinib had a weak effect on increasing the plasma exposure of repaglinide, a CYP2C8 substrate, indicating that tucatinib is a weak inhibitor of CYP2C8 in vivo. The geometric LS mean ratios (90% CIs) for AUCₗₐₛₜ, AUC_{0-∞}, and Cₘₐₓ for repaglinide dosed in combination with steady-state tucatinib compared to repaglinide dosed alone were 1.72 (1.55, 1.91), 1.69 (1.51, 1.90), and 1.69 (1.37, 2.10), respectively. These effects were statistically significant based on 90% CI values.

Tucatinib had no effect on the PK of tolbutamide, a CYP2C9 substrate, or the plasma exposure of the 4-hydroxytolbutamide metabolite of tolbutamide. The geometric LS mean ratios (90% CIs) for AUCₗₐₛₜ, AUC0-∞, and Cₘₐₓ for tolbutamide after tolbutamide/midazolam was dosed in combination with steady-state tucatinib compared to after tolbutamide/midazolam was dosed alone were 1.03 (1.01, 1.06), 1.05 (1.01, 1.09), and 0.961 (0.904, 1.02), respectively. The geometric LS mean ratios (90% CIs) for AUCₗₐₛₜ, AUC_{0-∞}, and Cₘₐₓ for 4-hydroxytolbutamide after tolbutamide/midazolam was dosed in combination with steady-state tucatinib compared to after tolbutamide/midazolam was dosed alone were 0.900 (0.868, 0.934), 0.918 (0.880, 0.958), and 0.881 (0.831, 0.934), respectively.

Tucatinib had a strong effect on increasing the plasma exposure of midazolam, a CYP3A4 substrate, indicating that tucatinib is a strong inhibitor of CYP3A4 in vivo. The geometric LS mean ratios (90% CIs) for AUCₗₐₛₜ, AUC_{0-∞}, and Cₘₐₓ for midazolam after steady-state tucatinib was dosed in combination with tolbutamide/midazolam compared to after tolbutamide/midazolam was dosed alone were 5.30 (4.65, 6.04), 5.74 (5.05, 6.53), and 3.01 (2.63, 3.45), respectively. These effects were statistically significant based on 90% CI values.

Tucatinib had no effect on the plasma exposure of 1-hydroxymidazolam based on AUCₗₐₛₜ and AUC_{0-∞}, but a weak effect on decreasing exposure based on Cₘₐₓ. The geometric LS mean ratios (90% CIs) for AUCₗₐₛₜ, AUC_{0-∞}, and Cₘₐₓ for 1-hydroxymidazolam after steady-state tucatinib was dosed in combination with tolbutamide/midazolam compared to after tolbutamide/midazolam was dosed alone were 0.945 (0.848, 1.05), 1.02 (0.903, 1.16), and 0.593 (0.507, 0.694), respectively. The effect on Cₘₐₓ was statistically significant based on 90% CI values.

Tucatinib had a strong effect on reducing the metabolism of midazolam; tucatinib decreased the geometric mean MR_{AUC0-∞} and MR_{Cmax} of 1-hydroxymidazolam by approximately 82.8% and 80.4%, respectively, following administration of steady-state tucatinib in combination with tolbutamide/midazolam compared to following administration of tolbutamide/midazolam alone.

Tucatinib had similar Tₘₐₓ and t_{1/2} values after single dosing (Day 4) and multiple dosing (Day 10), and the Rac of tucatinib after multiple dosing was 1.85. Similarly, ONT-993 had similar Tₘₐₓ and t_{1/2} values after single and multiple dosing, and the Rac of ONT-993 after multiple dosing was 2.09.

Part E: Tucatinib/Digoxin

Tucatinib had a weak effect on increasing the plasma exposures of digoxin, a P-gp substrate, based on AUCₗₐₛₜ and AUC_{0-∞} and a moderate effect on increasing plasma exposure based on Cₘₐₓ. The geometric LS mean ratios (90% CIs) for AUCₗₐₛₜ, AUC_{0-∞}, and Cₘₐₓ for digoxin dosed in combination with steady-state tucatinib compared to digoxin dosed alone were 1.53 (1.35, 1.74), 1.46 (1.29, 1.66), and 2.35 (1.90, 2.90), respectively. These effects were statistically significant based on 90% CI values. Overall, tucatinib is a weak inhibitor of P-gp in vivo.

Tucatinib had similar Tₘₐₓ and t12 values after single dosing (Day 8) and multiple dosing (Day 14), and the Rac of tucatinib after multiple dosing was 1.50. Similarly, ONT-993 had similar Tₘₐₓ and t_{1/2} values after single and multiple dosing, and the Rac of ONT-993 after multiple dosing was 2.01.

### Example 3: Assessment of Tucatinib and ONT-993 as Inhibitors of Human OAT2, OCT2, MATE1, and MATE2-K Mediated Transport

Substrate dependent inhibition is common among transporters including MATE1 and MATE2-K. In this study, creatinine was used as the probe substrate to assess the inhibitory potency of Tucatinib and ONT-993. OAT2, OCT2, OCT3, MATE1, and MATE2-K are the renal transporters identified to transport creatinine. Tucatinib and its metabolite ONT-993 were tested as potential inhibitors of OAT2, OCT2, MATE1, and MATE2-K mediated transport using creatinine as a probe substrate.

For this study, MDCK-II were maintained in DMEM with low glucose and 10% FBS. Cells were seeded at 60K±10K cells/well on 96-well, transwell membrane plates approximately 24 hours before transfection. Transport assays were carried out approximately 48 hours after transfection. Cells were transfected and treated to express the transporter of interest or treated with a control vector. The transport of creatinine was determined by radiometric detection.

Net transporter mediated uptake of the substrate by each transporter was calculated as follows: Net Transporter Mediated Substrate Uptake (pmol/min/cm²)=(Cellular accumulation in the presence of the transporter)−(Mean cellular accumulation in the absence of the transporter.

Percent inhibition was calculated as follows: Percent inhibition=100−(100*(transporter mediated uptake)_{with inhibition}/(transporter mediated uptake)_{without inhibition})

The following equation was used to determine IC50 values of uptake assays:

$$V = \frac{{V}_{0}}{1 + {( [ I ] / {IC}_{50} )}^{n}}$$

where V₀ is the mean transporter mediated flux in the absence of the test article, V is the transporter mediated flux in the presence of the test article throughout the concentration range tested, [I] is the inhibitor concentration, IC₅₀ represents the value at which transport is inhibited by 50%, and n is a Hill coefficient.

### Results

Using a range of concentrations up to 10 μM Tucatinib and ONT-993 for OAT2, the maximum inhibition of OAT2 mediated transport of creatinine was 14.7% and 44.9% at concentration of 10 μM for Tucatinib and ONT-993, respectively. Insufficient inhibition was observed to determine IC₅₀ values for Tucatinib and ONT-993 for OAT2.

Using a range of concentrations of 0.03 to 10 μM Tucatinib or ONT-993, concentration-dependent inhibitions of OCT2 mediated transport of creatinine were observed. The IC₅₀ values were determined to be 0.107±0.0379 μM for Tucatinib, and 0.544±0.278 μM for ONT-993 for OCT2.

Using a range of concentrations of 0.003 to 1 μM Tucatinib or 0.01 to 3 μM ONT-993, concentration-dependent inhibitions of MATE1 mediated transport of creatinine were observed. The IC₅₀ values were determined to be 0.0855±0.0175 μM for Tucatinib, and 0.0863±0.0126 μM for ONT-993 for MATE1.

Using a range of concentrations up to 10 μM Tucatinib and ONT-993, insufficient net creatinine uptake was observed to accurately determine the IC₅₀ value for Tucatinib or ONT-993 for MATE2-K.

### Example 4: Cytochrome P450 3A4/5 Inhibition in Human Liver Microsomes

This in vitro study was designed to evaluate the ability of tucatinib to inhibit the major CYP enzyme CYP3A4/5 in human liver microsomes (using two different substrates) with the aim of ascertaining the potential of tucatinib to inhibit the metabolism of concomitantly administered drugs. The inhibitory potency of tucatinib was determined in vitro by measuring the activity of CYP3A4/5 in human liver microsomes in the presence and absence of tucatinib. These in vitro experiments were designed to measure the concentration of inhibitor that causes 50% inhibition of marker substrate activity (IC₅₀ value) for direct, time- and metabolism-dependent inhibition of CYP3A4/5. Metabolism-dependent inhibition was further evaluated to determine how quickly (k_{inact} value) and to what extent (Ki value) tucatinib inactivates CYP3A4/5.

### Methods

Human liver microsomes from non-transplantable, donated livers were prepared and characterized. A mixed-gender pool of 200 individual human liver microsomal samples was used for this study (Sekisui XenoTech catalog number: H2620, lot number: 1210347).

To measure CYP activity, incubations were conducted at approximately 37° C. in 200 μL incubation mixtures (pH 7.4) containing water, potassium phosphate buffer (50 mM), MgCl₂ (3 mM), EDTA (1 mM), an NADPH-regenerating system (always the mixture of the following: NADP [1 mM], glucose 6 phosphate [5 mM], glucose 6 phosphate dehydrogenase [1 Unit/mL]), and marker substrate at the final concentrations indicated.

Aliquots of the stock and/or working solutions of tucatinib were manually added to buffer mixtures described above. Incubation mixtures were prepared in bulk to obviate the need for directly pipetting very small volumes (i.e., 1 μL or less). Incubations containing no tucatinib (0 μM; Solvent Control) contained the solvent used to dissolve tucatinib (i.e., DMSO).

The Tecan liquid handling system conducted all remaining steps for the IC₅₀ and Kₗ/k_{inact} determinations, with the exception of the centrifugation. For these assays, duplicate aliquots of the buffer mixtures were automatically added to 96-well plates at the appropriate locations. Aliquots of a substrate working solution were added to the 96-well plates prior to initiating reactions. Marker substrate reactions were initiated by the addition of an aliquot of an NADPH-regenerating system and were automatically terminated at approximately 5 min by the addition of the appropriate internal standard and stop reagent, acetonitrile. The samples were centrifuged at 920×g for 10 min at 10° C. The supernatant fractions were analyzed by LC-MS/MS. Standards were similarly prepared with the addition of authentic metabolite standards.

Due to the possibility that tucatinib may bind to microsomal protein or lipids, an attempt was made to design these experiments such that, in as many cases as possible, the microsomal protein, incubation time and buffer concentration were 0.1 mg/mL, 5 min and 50 mM, respectively.

To examine its ability to act as a direct inhibitor of enzymes, tucatinib (at concentrations ranging from 0.01 to 10 μM) was incubated with marker substrate and human liver microsomes. The concentrations of marker substrates were based on the Kₘ or S₅₀ data that were determined previously.

To examine its ability to act as a metabolism-dependent inhibitor of CYP3A4/5 enzymes, tucatinib (at the same concentrations used to evaluate direct inhibition) was preincubated at 37±2° C., in duplicate, with human liver microsomes and an NADPH-regenerating system for approximately 30 min. This preincubation allowed for the generation of intermediates that could inhibit human CYP3A4/5 activity. The preincubations were initiated by the addition of an aliquot of an NADPH-regenerating system. To examine its ability to act as a time-dependent inhibitor of CYP3A4/5 enzymes, additional duplicate samples at all tucatinib concentrations were preincubated for 30 min in the presence of pooled human liver microsomes, but in the absence of NADPH. This preincubation allowed assessment of whether any potential increase in inhibition was dependent upon NADPH (e.g., potentially CYP-mediated). Following the 30-min preincubation period, marker substrate was automatically added, and the incubations were continued to measure residual CYP enzyme activity. Incubations containing no tucatinib (0 μM; Solvent Control) and incubations that contained tucatinib but were not preincubated, served as negative controls.

Experiments were designed to further investigate the apparent metabolism-dependent inhibition of enzymes and determine the k_{inact} and K_{I} values for the inactivation of CYP3A4/5. All incubations were conducted with a Tecan Liquid Handling System.

To determine the k_{inact} and K_{I} values for the inactivation of CYP3A4/5, tucatinibwas preincubated in duplicate with pooled human liver microsomes at approximately 0.1 mg/mL and an NADPH-regenerating system for zero, 3, 6, 9, 15 and 30 min. After the preincubation, an aliquot of the preincubation mixture (20 μL) was transferred to a second tube containing the marker substrate, at approximately 10 times its Km, and an NADPH-regenerating system (final volume of 200 μL) resulting in a 10-fold dilution of the inhibitor to minimize the direct inhibitory effects of tucatinib and a 10 fold dilution of the microsomes. The incubation was then continued for 5 min to allow formation of any metabolites of the marker substrate. The residual CYP3A4/5 activities were measured.

Incubations containing tucatinib, but no probe substrate, were included to assess the possibility of analytical interference with substrate product (1′-hydroxymidazolam) by tucatinib and/or possible metabolite(s) in the analytical method.

Samples were analyzed by multiple reaction monitoring LC-MS/MS methods. Metabolites were quantified by reference to a standard calibration curve generated using the simplest appropriate weighting and regression algorithm. The regression fit was based on the peak area ratio of the analyte to internal standard calculated from calibration standard samples. Stock standard solutions and working solutions were prepared according to the custom Tecan script EVO Std-QC Spiking Solution Prep. Chromatographic peaks were integrated with Analyst Instrument Control and Data Processing Software (SCIEX, version 1.6.1).

### Results

Tucatinib directly inhibited CYP3A4/5-mediated midazolam 1′-hydroxylation with an IC₅₀ value of 3.3 μM. A maximum of 44% direct inhibition was observed for CYP3A4/5-mediated testosterone 6p-hydroxylation and so the associated IC₅₀ value was reported as >10 μM, the highest concentration of the test article evaluated. Tucatinib also caused metabolism-dependent inhibition, as the IC₅₀ values associated with CYP3A4/5 (as measured by midazolam 1′-hydroxylation and testosterone 6p-hydroxylation) decreased by factors of 2.71 and 2.13, respectively, after a 30-minute preincubation with NADPH.

The metabolism-dependent inhibition of CYP3A4/5 activity (as measured by midazolam 1′-hydroxylation) was further examined to measure the k_{inact} and K_{I} values associated with inactivation of this enzyme activity. Tucatinib inactivated CYP3A4/5-mediated midazolam 1′-hydroxylation with a mean±SE k_{inact} value of 0.011±0.001 min⁻¹ and a mean±SE K_{I} value of 0.54±0.25 μM. The efficiency of inactivation (k_{inact}/K_{I}) was 21 min⁻¹ mM⁻¹.

### Example 5: Evaluation of K; of Tucatinib for CYP2C8, CYP2C9, CYP3A4, and UGT1A1

The objective of this study was to evaluate in vitro the Kᵢ of tucatinib of the following human hepatic cytochrome P450 (CYP) enzymes (CYP2C8, CYP2C9, and CYP3A4) and UGT1A1.

Direct inhibition of tucatinib (0.1 to 25 μM) on CYP2C8, CYP2C9, CYP3A4, and UGT1A1 with corresponding marker substrate amodiaquine, diclofenac, midazolam, and β-estradiol at six concentrations (0.1×, 0.25×, 0.5×, 1×, 3×, and 5×Kₘ) was used to determine the inhibition constant (Kᵢ). The Kᵢ value of tucatinib for CYP2C8, CYP2C9, CYP3A4, and UGT1A1 was estimated as 0.170, 4.57, 0.805, and 1.81 μM, respectively. The inhibition mechanism was determined as competitive inhibition from all four in vitro assays.

Pooled human liver microsomes (HLM) from 150 individuals (79 males and 71 females) were obtained from BioreclamationIVT (Baltimore, Md.) and stored at approximately −70° C. The microsomes were characterized by the supplier for total protein and selected cytochrome P450 activities.

For Kᵢ determinations, incubations were conducted with up to eight concentrations of tucatinib (0.1, 0.22, 0.484, 1.07, 2.35, 5.16, 11.4, and 25 μM) and six concentrations of marker substrate (0.1, 0.25, 0.5, 1, 3, and 5×Kₘ). Incubation mixtures including HLM, tucatinib, marker substrate, and assay buffer [0.1 M potassium phosphate buffer containing 1 mM EDTA, pH 7.4 (CYP) or 0.05 M Tris buffer containing 150 mM potassium chloride and 10 mM magnesium chloride, pH 7.4 (UGT)] were pre-incubated at 37° C. for 10 minutes before initiation with the addition of pre-warmed NADPH [nicotinamide adenine dinucleotide phosphate, reduced form, 1 mM (CYP)] or UDPGA [uridine 5′-diphosphoglucuronic acid, 2 mM (UGT)]. The final organic solvent contribution was ≤1%. Incubations were terminated by the addition of chilled acetonitrile containing a stable isotope-labeled internal standard. Control incubations included a test article solvent control (no test article), positive control inhibitor, and an additional solvent control specific to the positive control inhibitor. All incubations were performed in triplicate. The inhibition constant (Kᵢ) was estimated.

Details of the incubation conditions for each assay are presented in the following table.

### Cytochrome P450 Activity Assays

| Assay | Substrate Km | Protein | Time | | Positive Control |
|---|---|---|---|---|---|
| (Enzyme Activity) | (μM) | (mg/mL) | (min) | Analyte | (μM) |
| Amodiaquine N-deethylase | 1.5 | 0.013 | 10 | Desethylamodiaquine | Montelukast (0.1) |
| (CYP2C8) | | | | | |
| Diclofenac 4′-hydroxylase | 6 | 0.1 | 5 | 4′-Hydroxydiclofenac | Sulfaphenazole (5) |
| (CYP2C9) | | | | | |
| Midazolam 1′-hydroxylase | 1.5 | 0.063 | 5 | 1′-Hydroxymidazolam | Ketoconazole (0.1) |
| (CYP3A4) | | | | | |
| β-Estradiol 3-glucuronidation | 20 | 0.2 | 20 or | β-Estradiol 3-(β-D- | Tangeretin (60) |
| (UGT1A1) | | | 5^{a} | glucuronide) | |

min Minutes.
Note:
The stopping solution was acetonitrile containing internal standard.
^{a}The incubation time for the substrate at 5 μM was 5 minutes (Deviation).

Samples were analyzed by liquid chromatography with tandem mass spectrometry (LC-MS/MS). Analyte concentrations were quantified by LC-MS/MS and interpolated from standard curves of authentic analyte. Standards and quality control samples were prepared in duplicate. Activities were calculated based on analyte concentration, incubation time, and protein concentration.

Enzymatic activity (pmol/minute/mg protein) was expressed as the formation of the analyte per final protein concentration per incubation time. Activity remaining was expressed as the enzymatic activity at each concentration point of the test article normalized by the solvent control mean activity.

Calculations were performed for competitive, noncompetitive, uncompetitive, and mixed inhibition (Deviation).

### Results

The inhibition constant (Kᵢ) of tucatinib on CYP2C8, CYP2C9, CYP3A4, and UGT1A1 was estimated as 0.170, 4.57, 0.805, and 1.81 μM, respectively. The inhibition mechanism was determined as competitive inhibition from all four in vitro assays.

What is claimed is:

1. A method for treating breast cancer in a subject comprising administering a therapeutically effective amount of tucatinib, or salt or solvate thereof, to the subject, wherein the subject is not concurrently receiving treatment with a therapeutically effective amount of a substrate of a multidrug and toxin extrusion (MATE) protein.

2. The method of claim 1, wherein the subject has not received treatment with the substrate of the MATE protein within the past 7 days.

3. The method of claim 1, wherein the subject has not received treatment with the substrate of the MATE protein within the past 3 months.

4. The method of claim 1, wherein the subject has not received treatment with the substrate of the MATE protein within the past 12 months.

5. The method of claim 1, wherein the subject has not previously received treatment with the substrate of the MATE protein.

6. The method of any one of claims 1-5, wherein the MATE protein is MATE1.

7. The method of any one of claims 1-5, wherein the MATE protein is MATE2K.

8. The method of anyone of claims 1-7, wherein the substrate of the MATE protein is selected from the group consisting of metformin, oxazolidinone, fexofenadine, tetraethylammonium (TEA), N-methylphenylpyridinium (MPP+), paraquat, agmatine, cimetidine, procainamide, pramipexole, atenolol, serotonin, quinidine, verapamil, cisplatin, oxaliplatin, and pyrimethamine.

9. The method of claim 8, wherein the substrate is metformin.

10. A method for treating breast cancer in a subject comprising administering a therapeutically effective amount of tucatinib, or salt or solvate thereof, to the subject, wherein the subject is not concurrently receiving treatment with a therapeutically effective amount of a substrate of an organic cation transporter (OCT).

11. The method of claim 10, wherein the subject has not received treatment with the substrate of the OCT within the past 7 days.

12. The method of claim 10, wherein the subject has not received treatment with the substrate of the OCT within the past 3 months.

13. The method of claim 10, wherein the subject has not received treatment with the substrate of the OCT protein within the past 12 months.

14. The method of claim 10, wherein the subject has not previously received treatment with the substrate of the OCT.

15. The method of any one of claims 10-14, wherein the OCT is OCT1.

16. The method of any one of claims 10-14, wherein the OCT is OCT2.

17. The method of anyone of claims 10-16, wherein the substrate of the OCT is selected from the group consisting of metformin, oxazolidinone, fexofenadine, tetraethylammonium (TEA), N-methylphenylpyridinium (MPP+), paraquat, agmatine, cimetidine, procainamide, pramipexole, atenolol, serotonin, quinidine, verapamil, cisplatin, oxaliplatin, and pyrimethamine.

18. The method of claim 17, wherein the substrate is metformin.

19. The method of any one of claims 10-18, wherein the subject is not concurrently receiving treatment with a therapeutically effective amount of a substrate of a MATE protein.

20. The method of claim 19, wherein the subject has not received treatment with the substrate of the MATE protein within the past 7 days.

21. The method of claim 19, wherein the subject has not received treatment with the substrate of the MA TE protein within the past 3 months.

22. The method of claim 19, wherein the subject has not received treatment with the substrate of the MATE protein within the past 12 months.

23. The method of claim 19, wherein the subject has not previously received treatment with the substrate of the MATE protein.

24. The method of any one of claims 19-23, wherein the MATE protein is MATE1.

25. The method of any one of claims 19-23, wherein the MATE protein is MATE2K.

26. A method for treating breast cancer in a subject comprising administering a therapeutically effective amount of tucatinib, or salt or solvate thereof, to the subject, wherein the subject does not have impaired renal function.

27. The method of claim 26, wherein the subject has not had impaired renal function within the past 12 months.

28. The method of any one of claims 1-25, wherein the subject does not have impaired renal function.

29. The method of claim 28, wherein the subject has not had impaired renal function within the past 12 months.

30. The method of any one of claims 26-29, wherein impaired renal function is determined based on the serum creatinine level in the subject.

31. The method of claim 30, wherein a) the subject is male and the subject has a serum creatinine level of less than 1.5 mg/dL or b) the subject is female and has a serum creatinine level of less than to 1.4 mg/dL.

32. The method of any one of claims 26-29, wherein impaired renal function is determined based on the subject having abnormal creatinine clearance.

33. The method of any one of claims 26-29, wherein impaired renal function is determined based on the glomerular filtration rate of the subject.

34. The method of any one of claims 1-33, wherein the subject is not concurrently receiving treatment with a therapeutically effective amount of a compound that modulates the activity of a cytochrome p450 protein.

35. The method claim 34, wherein the subject has not received treatment with the compound that modulates the activity of the cytochrome p450 protein within the past 7 days.

36. The method of claim 34, wherein the subject has not received treatment with the compound that modulates the activity of the cytochrome p450 protein within the past 3 months.

37. The method of claim 34, wherein the subject has not received treatment with the compound that modulates the activity of the cytochrome p450 protein within the past 12 months.

38. The method of claim 34, wherein the subject has not previously received treatment with compound that modulates the activity of the cytochrome p450 protein.

39. The method of any one of claims 34-38, wherein the compound that modulates the activity of the cytochrome p450 protein is an inhibitor of the activity of the cytochrome p450 protein.

40. The method of claim 39, wherein the compound that modulates the activity of the cytochrome p450 protein is a strong inhibitor of the activity of the cytochrome p450 protein.

41. The method of claim 39 or 40, wherein the cytochrome p450 protein is CYP3A4.

42. The method of claim 41, wherein the compound that inhibits the activity of CYP3A4 is itraconazole.

43. The method of claim 39 or 40, wherein the cytochrome p450 protein is CYP2C8.

44. The method of claim 43, wherein the compound that inhibits the activity of CYP2C8 is gemfibrozil.

45. The method of any one of claims 34-38, wherein the compound that modulates the activity of the cytochrome p450 protein is an inducer of the activity of the cytochrome p450 protein.

46. The method of claim 45, wherein the compound that modulates the activity of the cytochrome p450 protein is a strong inducer of the activity of the cytochrome p450 protein.

47. The method of claim 45 or 46, wherein the cytochrome p450 protein is CYP3A4.

48. The method of claim 47, wherein the cytochrome p450 protein is CYP2C8.

49. The method of any one of claims 45-48, wherein the compound that induces the activity of the cytochrome p450 protein is rifampin.

50. A method for treating breast cancer in a subject comprising administering a therapeutically effective amount of tucatinib, or salt or solvate thereof, to the subject, wherein the subject is not concurrently receiving treatment with a therapeutically effective amount of a compound that modulates the activity of a cytochrome p450 protein.

51. The method of claim 50, wherein the subject has not received treatment with the compound that modulates the activity of the cytochrome p450 protein within the past 7 days.

52. The method of claim 50, wherein the subject has not received treatment with the compound that modulates the activity of the cytochrome p450 protein within the past 3 months.

53. The method of claim 50, wherein the subject has not received treatment with the compound that modulates the activity of the cytochrome p450 protein within the past 12 months.

54. The method of claim 50, wherein the subject has not previously received treatment with compound that modulates the activity of the cytochrome p450 protein.

55. The method of any one of claims 50-54, wherein the compound that modulates the activity of the cytochrome p450 protein is an inhibitor of the activity of the cytochrome p450 protein.

56. The method of claim 55, wherein the compound that modulates the activity of the cytochrome p450 protein is a strong inhibitor of the activity of the cytochrome p450 protein.

57. The method of claim 55 or 56, wherein the cytochrome p450 protein is CYP3A4.

58. The method of claim 57, wherein the compound that inhibits the activity of CYP3A4 is itraconazole.

59. The method of claim 55 or 56, wherein the cytochrome p450 protein is CYP2C8.

60. The method of claim 59, wherein the compound that inhibits the activity of CYP2C8 is gemfibrozil.

61. The method of any one of claims 50-54, wherein the compound that modulates the activity of the cytochrome p450 protein is an inducer of the activity of the cytochrome p450 protein.

62. The method of claim 61, wherein the compound that modulates the activity of the cytochrome p450 protein is a strong inducer of the activity of the cytochrome p450 protein.

63. The method of claim 61 or 62, wherein the cytochrome p450 protein is CYP3A4.

64. The method of claim 61 or 62, wherein the cytochrome p450 protein is CYP2C8.

65. The method of any one of claims 61-64, wherein the compound that induces the activity of the cytochrome p450 protein is rifampin.

66. A method for treating breast cancer in a subject comprising administering a therapeutically effective amount of tucatinib, or salt or solvate thereof, to the subject, wherein the subject is not concurrently receiving treatment with a therapeutically effective amount of a substrate of a cytochrome p450 protein.

67. The method of claim 66, wherein the subject has not received treatment with the substrate of the cytochrome p450 protein within the past 7 days.

68. The method of claim 66, wherein the subject has not received treatment with the substrate of the cytochrome p450 protein within the past 3 months.

69. The method of claim 66, wherein the subject has not received treatment with the substrate of the cytochrome p450 protein within the past 12 months.

70. The method of claim 66, wherein the subject has not previously received treatment with substrate of the cytochrome p450 protein.

71. The method of any one of claims 66-70, wherein the cytochrome p450 protein is CYP3A4.

72. The method of any one of claims 66-71, wherein the substrate of the cytochrome p450 protein is a sensitive CYP3A substrate.

73. The method of any one of claims 66-70, wherein the cytochrome p450 protein is CYP2C8.

74. The method of any one of claims 66-73, wherein the substrate of the cytochrome p450 protein is selected from the group consisting of budesonide, buspirone, eplerenone, eletriptan, felodipine, fluticasone, lovastatin, midazolam, saquinavir, sildenafil, simvastatin, triazolam, and vardenafil.

75. The method of claim 74, wherein the substrate of the cytochrome p450 protein is midazolam.

76. A method for treating breast cancer in a subject comprising administering a therapeutically effective amount of tucatinib, or salt or solvate thereof, to the subject, wherein the subject is not concurrently receiving treatment with a therapeutically effective amount of a substrate of P-glycoprotein (P-gp).

77. The method of claim 76, wherein the subject has not received treatment with the substrate of P-gp within the past 7 days.

78. The method of claim 76, wherein the subject has not received treatment with the substrate of P-gp within the past 3 months.

79. The method of claim 76, wherein the subject has not received treatment with the substrate of P-gp within the past 12 months.

80. The method of claim 76, wherein the subject has not previously received treatment with substrate of P-gp.

81. The method of any one of claims 76-80, wherein the substrate of P-gp is a substrate with a narrow therapeutic index.

82. The method of any one of claims 76-81, wherein the substrate of P-gp is selected from the group consisting of amitriptyline, carbamazepine, clonidine, cyclosporine, digitoxin, digoxin, imipramine, phenobarbital, phenytoin, quinidine, rifampicin, sirolimus, tacrolimus, temsirolimus, trimipramine, vincristine, paclitaxel, and dabigatran etexilate.

83. The method of claim 82, wherein the substrate of P-gp is digoxin.

84. The method of any one of claims 1-83, wherein the tucatinib is administered to the subject at a dose of about 150 mg to about 650 mg.

85. The method of claim 84, wherein the tucatinib is administered to the subject at a dose of about 300 mg.

86. The method of claim 84 or 85, wherein the tucatinib is administered once or twice per day.

87. The method of claim 86, wherein the tucatinib is administered to the subject at a dose of about 300 mg twice per day.

88. The method of any one of claims 1-87, wherein the tucatinib is administered to the subject orally.

89. The method of any one of claims 1-88, wherein the breast cancer is a HER2 positive breast cancer.

90. The method of claim 89, wherein the cancer is determined to be HER2 positive using in situ hybridization, fluorescence in situ hybridization, or immunohistochemistry.

91. The method of any one of claims 1-90, wherein the breast cancer is metastatic.

92. The method of claim 91, wherein the breast cancer has metastasized to the brain.

93. The method of any one of claims 1-92, wherein the breast cancer is locally advanced.

94. The method of any one of claims 1-93, wherein the breast cancer is unresectable.

95. The method of any one of claims 1-94, further comprising administering one or more additional therapeutic agents to the subject to treat the breast cancer.

96. The method of claim 95, wherein the one or more additional therapeutic agents is selected from the group consisting of capecitabine and an anti-HER2 antibody.

97. The method of claim 95, wherein the one or more additional therapeutic agents is capecitabine.

98. The method of claim 95, wherein the one or more additional therapeutic agents is trastuzumab.

99. The method of claim 95, wherein the one or more additional therapeutic agents are capecitabine and trastuzumab.

100. The method of claim 97 or 99, wherein the capecitabine is administered to the subject at a dose of about 500 mg/m² to about 1500 mg/m².

101. The method of claim 100, wherein the capecitabine is administered to the subject at a dose of about 1000 mg/m².

102. The method of claim 100 or 101, wherein the capecitabine is administered to the subject orally.

103. The method of any one of claims 99-102, wherein the capecitabine is administered to the subject twice per day.

104. The method of claim 98 or 99, wherein the trastuzumab is administered to the subject at a dose of about 400 mg to about 800 mg.

105. The method of claim 104, wherein the trastuzumab is administered to the subject at a dose of about 600 mg

106. The method of claim 104 or 105, wherein the trastuzumab is administered to the subject subcutaneously.

107. The method of claim 98 or 99, wherein the trastuzumab is administered to the subject at a dose of about 4 mg/kg to about 10 mg/kg.

108. The method of claim 107, wherein the trastuzumab is administered to the subject at a dose of about 6 mg/kg.

109. The method of claim 107, wherein the trastuzumab is administered to the subject at a dose of about 8 mg/kg.

110. The method of claim 107, wherein the trastuzumab is administered to the subject at an initial dose of about 8 mg/kg followed by subsequent doses of about 6 mg/kg.

111. The method of any one of claims 107-110, wherein the trastuzumab is administered intravenously.

112. The method of any one of claims 104-111, wherein the trastuzumab is administered once about every 1 week, once about every 2 weeks, once about every 3 weeks, or once about every 4 weeks.

113. The method of claim 112, wherein the trastuzumab is administered once about every 3 weeks.

114. The method of claim 99, wherein the tucatinib, capecitabine and trastuzumab are administered to the subject on a 21 day treatment cycle.

115. The method of claim 114, wherein the tucatinib is administered to the subject twice per day on each day of the 21 day treatment cycle.

116. The method of claim 114 or 115, wherein the capecitabine is administered to the subject twice per day on each of days 1-14 of the 21 day treatment cycle.

117. The method of any one of claims 114-116, wherein the trastuzumab is administered to the subject once per 21 day treatment cycle.

118. The method of claim 117, wherein the dose of trastuzumab during the first 21 day treatment cycle is 8 mg/kg and the dose of trastuzumab during the subsequent 21 day treatment cycles is 6 mg/kg.

119. The method of any one of claims 1-118, wherein the subject has been previously treated with one or more additional therapeutic agents for the breast cancer.

120. The method of claim 119, wherein the one or more additional therapeutic agents is an anti-HER2 antibody or anti-HER2 antibody-drug conjugate.

121. The method of claim 120, wherein the one or more additional therapeutic agents is trastuzumab, pertuzumab and/or T-DM1.

122. The method of any one of claims 1-121, wherein the subject has not been treated with another therapeutic agent for the breast cancer within the past 12 months.

123. The method of any one of claims 1-118, wherein the subject has not previously been treated with another therapeutic agent for the breast cancer.

124. The method of any one of claims 1-123, wherein the subject has not previously been treated with lapatinib, neratinib, afatinib, or capecitabine.

125. The method of any one of claims 1-124, wherein treating the subject results in a tumor growth inhibition (TGI) index of at least about 85%.

126. The method of any one of claims 1-124, wherein treating the subject results in a TGI index of about 100%.

127. The method of any one of claims 1-126, wherein one or more therapeutic effects in the subject is improved after administration of tucatinib to the subject relative to a baseline.

128. The method of claim 127, wherein the one or more therapeutic effects is selected from the group consisting of: size of a tumor derived from the breast cancer, objective response rate, duration of response, time to response, progression free survival and overall survival.

129. The method of any one of claims 1-128, wherein the size of a tumor derived from the breast cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the breast cancer before administration of tucatinib to the subject.

130. The method of any one of claims 1-129, wherein the objective response rate is at least about 20%, at least about 25%, at least about 30%, at least about 35%, at least about 40%, at least about 45%, at least about 50%, at least about 60%, at least about 70%, or at least about 80%.

131. The method of any one of claims 1-130, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of tucatinib to the subject.

132. The method of any one of claims 1-131, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of tucatinib to the subject.

133. The method of any one of claims 1-132, wherein the duration of response to tucatinib is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of tucatinib to the subject.

134. The method of any one of claims 1-133, wherein the subject is a human.
